(19) United States
(12) Patent Application Publication
(10) Pub. No.: US 2010/0286211 A1
(43) Pub. Date: Nov. 11, 2010

(54) OXAZOLIDINONE DERIVATIVES AS ANTIMICROBIALS

(76) Inventors: Biswajit Das, Haryana (IN); Shahadal Ahmed, Haryana (IN); Ajay Singh Yadav, Haryana (IN); Soma Ghosh, Haryana (IN); Arti Gujrati, New Delhi (IN); Pankaj Sharma, New Delhi (IN); Ashok Rattan, Haryana (IN)

Correspondence Address: Ranbaxy Inc., Intellectual Property Department, 600 College Road East, PRINCETON, NJ, 08540, US

(21) Appl. No.: 11/576,635

(22) PCT Filed: Oct. 6, 2005
(86) PCT No.: PCT/IB05/02971

§ 371 Date: Jun. 10, 2008

Related U.S. Application Data
(60) Provisional application No. 60/616,964, filed on Oct. 8, 2004.

(51) Int. Cl. A61K 31/4439; A61K 31/433; A61K 31/421; C07D 417/14; A61K 31/4245; C07D 263/20; C07D 413/14; A61K 31/422; A61P 31/04

(52) U.S. Cl. 514/341; 514/363; 514/364; 514/376; 546/271.4; 548/136; 548/143; 548/232

(57) ABSTRACT

The present invention relates to certain substituted phenyl oxazolidinones and to processes for the synthesis of the same. This invention also relates to pharmaceutical compositions containing the compounds of the present invention as antimicrobials. The compounds are useful antimicrobial agents, effective against a number of human and veterinary pathogens, including gram-positive aerobic bacteria, for example, multiple-resistant staphylococci, streptococci and enterococci as well as anaerobic organisms, for example, Bacterioides spp. and Clostridia spp. species, and acid fast organisms, for example, Mycobacterium tuberculosis, Mycobacterium avium and Mycobacterium spp.

### FIELD OF THE INVENTION

The present invention relates to certain substituted phenyl oxazolidinones and to processes for the synthesis of the same. This invention also relates to pharmaceutical compositions containing the compounds of the present invention as antimicrobials. The compounds are useful antimicrobial agents, effective against a number of human and veterinary pathogens, including gram-positive aerobic bacteria, for example, multiple-resistant staphylococci, streptococci and enterococci as well as anaerobic organisms, for example, Bacterioides spp. and Clostridia spp. species, and acid fast organisms, for example, Mycobacterium tuberculosis, Mycobacterium avium and Mycobacterium spp.

### BACKGROUND OF THE INVENTION

Increasing antibacterial resistance in Gram-positive bacteria has presented a formidable treatment problem. The enterococci, although traditionally not virulent pathogens, have been shown, when associated with vancomycin resistance, to have an attributable mortality of approximately 40%. Staphylococcus aureus, the traditional pathogen of postoperative wounds, has been resistant to penicillin due to production of penicillinases. This resistance was overcome by the development of various penicillinase-stable β lactams. But the pathogen responded by synthesizing modified target penicillin binding protein-2′ leading to less affinity for β lactam antibiotics and a phenotype known as Methicillin Resistant S. aureus (MRSA). These strains, until recently were susceptible to vancomycin, which despite its various drawbacks, has become the drug of choice for MRSA infections. Streptococcus pneumoniae is a major pathogen causing pneumonia, sinusitis and meningitis. Until very recently it was highly susceptible to penicillin. Recently though, different PBP 2′ strains with different susceptibility to penicillin have been reported from across the globe.

Oxazolidinones are a class of synthetic antimicrobial agents which kill Gram-positive pathogens by inhibiting a very early stage of protein synthesis. Oxazolidinones inhibit the formation of ribosomal initiation complex involving 30S and 50S ribosomes leading to prevention of initiation complex formation. Due to their mechanism of action, these compounds are active against pathogens resistant to other clinically useful antibiotics.

WO 04/056817 discloses oxazolidinone derivatives and their uses as antimicrobial agents. WO 04/056818 discloses substituted oxazolidinone derivatives described as antimicrobial agents. WO 04/14392 discloses substituted phenyl oxazolidinone derivatives which are described as antimicrobials. WO 03/97059 discloses polymorphic forms of phenyl oxazolidinone derivatives. WO 03/08389 discloses substituted phenyl oxazolidinone derivatives which are described as potential antimicrobials. WO 03/07870 discloses oxazolidinone derivatives described as antimicrobials. WO 04/14392 discloses substituted phenyl oxazolidinone derivatives described as antimicrobials. WO 93/09103 discloses substituted aryl and heteroaryl phenyl oxazolidinone said to be useful as antibacterial agents. WO 98/54161 and U.S. Pat. No. 6,255,304 disclose oxazolidinone antibacterial agents having a thiocarbonyl functionality. WO00/29396 discloses substituted phenyloxazolidinones derivatives for antibacterial medicament for treating human being and animals.

WO 01/80841 discloses the use of thioamide oxazolidinones for the treatment of bone resorption and osteoporosis. WO 01/94342 and U.S. Pat. No. 6,689,779 disclose oxazolidinone derivatives having pyridine or pyrimidine moieties and a process for the preparation thereof. WO 03/022824 discloses oxazolidinone and/or isoxazoline as antibacterial agent. WO 03/072553 discloses N-aryl-2-oxazolidinone-5-carboxamides and their derivatives and their use as antibacterial agents. WO03/006447 discloses oxazolidinone compounds having thiocarbonyl functionality that are described as antibacterial agents.

U.S. Pat. No. 5,565,571, U.S. Pat. No. 5,801,246, U.S. Pat. No. 5,756,732, U.S. Pat. No. 5,654,435, and U.S. Pat. No. 5,654,428 disclose substituted aryl and heteroaryl phenyloxazolidinones, which are described as useful as antibacterial agents.

However, in view of the above, there remains a need for novel oxazolidinone derivatives that can be effective antimicrobials.

### SUMMARY OF THE INVENTION

Herein are provided oxazolidinone derivatives, which have a good activity against multiply resistant Gram-positive pathogens like methicilline resistant Staphylococcus aureus (MRSA), vancomycin-resistant Enterococci (VRE) and Streptococcus pneumonia. Some of these molecules have activity against multiple drug resistant tuberculosis (MDR-TB) strain, while others have significant activity against important anaerobic bacteria.

Herein are provided phenyloxazolidinone derivatives that exhibit good antibacterial activity against Gram-positive pathogens like MRSA, VRE and PRSP against MDR-TB and MAI sirens and Gram-negative pathogens like Morazella catarrhalis and Haemophilus influenza in order to provide safe and effective treatment of bacterial infection.

In one aspect, provided are compounds having the structure of Formula I,

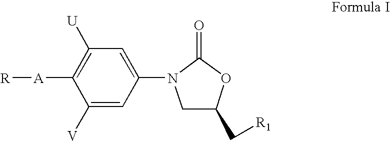

and their pharmaceutically acceptable salts, pharmaceutically acceptable solvates, esters, enantiomers, diastereomers, N-oxides, polymorphs, prodrugs or metabolites, wherein
A can be

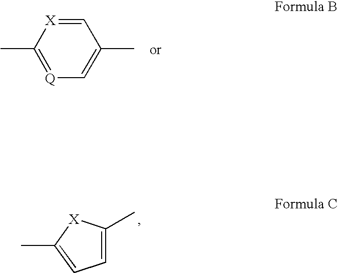

wherein Q and X can be independently selected from —N—, —O—, —C—F, —CH— or —S—;
U and V can be independently selected from hydrogen, lower (C₁₋₆) alkyl or halogen,
   wherein both U and V cannot be H at the same time
R can be CH═NOR_{f}, CH═NOC(═O)R_{f}, CH═NOSO₂R_{f}, CH═NOC(═O)NHR_{f}, heterocyclyl or heteroaryl, wherein
   R_{f} can be hydrogen, alkyl, alkenyl, alkynyl, alkoxy, cycloalkyl, aryl, aralkyl, heteroaryl, heterocyclyl, heteroarylalkyl or heterocyclylalkyl;
R₁ can be azido, NCS, NHYR_{f}, NRⱼ, C(=T)NR_{f}R_{q}, NR_{f}R_{q}, NRⱼ(C═O)ORₛ, wherein
   Y can be (C═O), (C═S) or SO₂,
   R_{f} is the same as defined earlier,
   T can be O, S, —N(CN), —N(NO₂), —CH(NO₂),
   Rⱼ can be hydrogen, alkyl, alkenyl, cycloalkyl, aryl, aralkyl, heterocyclyl, heteroaryl, heteroarylalkyl or heterocyclylalkyl,
   R_{q} can be hydrogen, alkyl, cycloalkyl, aryl, aralkyl, heteroaryl, heterocyclyl, heteroarylalkyl or heterocyclylalkyl, and
   Rₛ can be alkyl, alkenyl, alkynyl, cycloalkyl, aryl, aralkyl, heteroarylalkyl or heterocyclylalkyl;
with the proviso that:
   a when U is H, V is F, R₁ is NHCOCH₃ and A is Formula B (wherein Q or X is N), then R can be a five membered heteroaryl ring containing two or four N atoms (wherein the five membered heteroaryl ring containing four N atom is linked through N-atom to Formula B and is always substituted);
   when A is Formula B (wherein Q and X both are N) and U,V and R₁ are as defined above then R cannot be a five membered heterocyclyl ring containing 2 hetero atoms.

Such compounds can include one or more of the following embodiments. R₁ can be, for example, amino, isothiocyanate, tert-butyl isoxazol-3-yl carbamate, isoxzol-3-amine, ethanethioamido, acetamide, thiourea, N-methylthiourea or methyl carbamate. V and U can be independently selected from, for example, hydrogen or fluorine. A can be substituted heteroaryl, for example, pyridinyl, monofluorophenyl, pyrimidinyl, furanyl or thiophenyl. R can be optionally substituted heteroaryl, for example, 2-methyl-2H-tetrazolyl, 1-methyl-1H-tetrazolyl, 1H-1,2,4-triazolyl, 1,3-oxazolyl, 1H-imidazolyl, 5-phenyl-1H-tetrazolyl, 3a,7a-dihydro-1H-benzimidazolyl, 3-(1H-imidazol-4-yl)pyridine, oxazol-5-yl methanol, 5-methyl-5-tetrazole, (5R)-5-(hydroxymethyl)-1,3-oxazolidin-2-one, 1-methyl-2-phenyl-1H-imidazole, 1,3,4-thiazol-2-amine, 2-methyl-1,3,4-oxadiazole, N-1,3,4-thaidiazole-2-yl acetamide, 1H-pyrrol-3-yl methanol, 1H-pyrrole-3-carbaldehyde, 1H-pyrrole-3-carbaldehyde oxime, (1E)-acetaldehyde O-(3,4-difluororbenzyl)oxime, (1E)-acetaldehyde O-acetyloxime, (1E)-acetaldehyde O-benzoyl oxime, (1Z)-acetaldehyde-O -({[4-(trifluoromethyl)-phenyl]-amino}carbonyl) oxime, (1E)-acetaldehyde O-[(tert-butyl amino)-carbonyl]-oxime or (1Z)-acetaldehyde-O-{[(4-fluorophenyl)amino]carbonyl}-oxime.

In other embodiments, provided are compounds that include, for example,
N-[((5S)-3-{3,5-difluoro-4-[6-(3-formyl-1H-pyrrol-1-yl)pyridin-3-yl]phenyl}-2-oxo-1,3-oxazolidin-5-yl)methyl]acetamide (Compound No. 1)
N-{[(5S)-3-(3,5-difluoro-4-{6[-3-(hydroxymethyl)-1H-pyrrol-1-yl]pyridin-3-yl]phenyl)-2-oxo-1,3-oxazolidin-5-yl}methyl}acetamide (Compound No. 2)
N-({(5S)-3-[3-fluoro-4-(2-{3-[(E)-(hydroxyimino)methyl]-1H-pyrrol-1-yl}pyrimidin-5-yl)phenyl]-2-oxo-1,3-oxazolidin-5-yl}methyl)acetamide. (Compound No. 3)
N-({(5S)-3-[3,5-difluoro-4-(2-{3-[(E)-(hydroxyimino)methyl]-1H-pyrrol-1-yl}pyrimidin-5-yl)phenyl]-2-oxo-1,3-oxazolidin-5-yl}methyl)acetamide. (Compound No. 4),
N-({(5S)-3-[4′-((E)-{[(3,4-difluorobenzyl)oxy]imino}methyl)-2,3′,6-trifluorobiphenyl-4-yl]-2-oxo-1,3-oxazolidin-5-yl}methyl)acetamide (Compound No. 5)
N-{[(5S)-3-(4′-{(E)-[(acetyloxy)imino]methyl}-2,3′,6-trifluorobiphenyl-4-yl)-2-oxo-1,3-oxazolidin-5-yl]methyl}acetamide (Compound No. 6)
N-{[(5S)-3-(4′-{(E)-[(benzoyloxy)imino]methyl}-2,3′,6-trifluorobiphenyl-4-yl)-2-oxo-1,3-oxazolidin-5-yl]methyl}acetamide (Compound No. 7)
N-({(5S)-2-oxo-3-[2,3′,6-trifluoro-4′-(E)-{[(methylsulfonyl)oxy]imino}methyl)biphenyl-4-yl]-1,3-oxazolidin-5-yl}methyl)acetamide (Compound No. 8)
N-({(5S)-2-oxo-3-[2,3′,6-trifluoro-4′-(E)-{[({[4-(trifluoromethyl)phenyl]amino}carbonyl)oxy]imino}methyl)biphenyl-4-yl]-1,3-oxazolidin-5-yl}methyl)acetamide (Compound No. 9)
N-[((5S)-3-{4′-[(E)-({[(tent-butylamino)carbonyl]oxy}imino)methyl]-2,3′,6-trifluorobiphenyl-4-yl}-2-oxo-1,3-oxazolidin-5-yl)methyl]acetamide (Compound No. 10)
N-{[(5S)-2-oxo-3-(2,3′,6-trifluoro-4′-{(E)-[({[(4-fluorophenyl)amino]carbonyl}oxy) imino]methyl}biphenyl-4-yl)-1,3-oxazolidin-5-yl]methyl}acetamide (Compound No. 11)
N-({(5S)-2-oxo-3-[2,3′,6-trifluoro-4′-(1,3-oxazol-5-yl)biphenyl-4-yl]-1,3-oxazolidin-5-yl}methyl)acetamide (Compound No. 12),
N-[((5S)-3-{3,5-difluoro-4-[6-(1H-1,2,4-triazol-1-yl)pyridin-3-yl]phenyl}-2-oxo-1,3-oxazolidin-5-yl)methyl]acetamide (Compound No. 13),
N-{[(5S)-3-(3,5-difluoro-4-{6-[5-(1,3-oxazol-4-yl)-2-furyl]pyridin-3-yl}phenyl)-2-oxo-1,3-oxazolidin-5-yl]methyl}acetamide (Compound No. 14),
N-{[(5S)-3-(3,5-difluoro-4-{6-[5-(1,3-oxazol-4-yl)-2-thienyl]pyridin-3-yl}phenyl)-2-oxo-1,3-oxazolidin-5-yl]methyl}acetamide (Compound No. 15),
N-{[(5S)-3-(3,5-difluoro-4-{6-[3-(hydroxymethyl)-1H-pyrrol-1-yl]pyridin-3-yl}phenyl)-2-oxo-1,3-oxazolidin-5-yl]methyl}acetamide (Compound No. 16),
N-({(5S)-3-[3,5-difluoro-4-(6-{3-[(E)-(hydroxyimino)methyl]-1H-pyrrol-1-yl]pyridin-3-yl}phenyl]-2-oxo-1,3-oxazolidin-5-yl]methyl}acetamide (Compound No. 17),
N-({(5S)-3-[2,3′-difluoro-4′-5-methyl-1,2,4-oxadiazol-3-yl)biphenyl-4-yl]-2-oxo-1,3-oxazolidin-5-yl}methyl)acetamide (Compound No. 18),
N-({(5S)-2-oxo-3-[2,3′,6-trifluoro-4′-(5-methyl-1,2,4-oxadiazol-3-yl)biphenyl-4-yl]-1,3-oxazolidin-5-yl}methyl)acetamide (Compound No. 19),
N-[((5S)-3-{3,5-difluoro-4-[6-(2-methyl-2H-tetrazol-5-yl)pyridin-3-yl]phenyl}-2-oxo-1,3-oxazolidin-5-yl)methyl]acetamide (Compound No. 20),
N-[((5S)-3-{3,5-difluoro-4-[6-(1H-imidazol-1-yl)pyridin-3-yl]phenyl}-2-oxo-1,3-oxazolidin-5-yl)methyl]acetamide (Compound No. 21),
N-[((5S)-3-{3,5-difluoro-4-[6-(1-methyl-1H-tetrazol-5-yl)pyridin-3-yl]phenyl}-2-oxo-1,3-oxazolidin-5-yl)methyl]acetamide (Compound No. 22),
(5S)-3-{3,5-difluoro-4-[6-(2-methyl-2H-tetrazol-5-yl)pyridin-3-yl]phenyl}-5-[(1,3-thiazol-2-ylamino)methyl]-1,3-oxazolidin-2-one (Compound No. 23),
N-{[(5S)-3-(2,3′-difluoro-4′-{3-[(E)-(hydroxyimino)methyl]-1H-pyrrol-1-yl}biphenyl-4-yl)-2-oxo-1,3-oxazolidin-5-yl]methyl}acetamide (Compound No. 24),
N-({(5S)-3-[2,3′-difluoro-4′-(5-methyl-1H-tetrazol-1-yl)biphenyl-4-yl]-2-oxo-1,3-oxazolidin-5-yl}methyl)acetamide (Compound No. 25),
N-((S)-3-{3,5-Difluoro-4-[6-[5-methyl-[1,3,4]oxadiazol-2-yl)-pyridin-3-yl]-phenyl}-2-oxo-oxazolidin-5-ylmethyl)-acetamide (Compound No. 26),
N-((S)-3-{4-[6-(5-Amino-[1,3,4]thiadiazol-2-yl)-pyridin-3-yl]-3,5-difluoro-phenyl}-2-oxo-oxazolidin-5-ylmethyl)-acetamide (Compound No. 27),
N-({(5S)-3-[4′-(1H-benzimidazol-2-yl)-2,3′,6-trifluorobiphenyl-4-yl]-2-oxo-1,3-oxazolidin-5-yl}methyl)acetamide (Compound No. 28),
N-[((5S)-3-{3,5-difluoro-4-[6-(1H-1,2,3-triazol-1-yl)pyridin-3-yl]phenyl}-2-oxo-1,3-oxazolidin-5-yl)methyl]acetamide (Compound No. 29),
N-[((5S)-3-{3,5-difluoro-4-[6-(4-phenyl-1H-imidazol-1-yl)pyridin-3-yl]phenyl}-2-oxo-1,3-oxazolidin-5-yl)methyl]acetamide (Compound No. 30),
N-[((5S)-3-{3-fluoro-4-[5-(2-methyl-2H-tetrazol-5-yl)-2-furyl]phenyl}-2-oxo-1,3-oxazolidin-5-yl)methyl]acetamide (Compound No. 31),
N-[((5S)-3-{3-fluoro-4-[5-(1-methyl-1H-tetrazol-5-yl)-2-furyl]phenyl}-2-oxo-1,3-oxazolidin-5-yl)methyl]acetamide (Compound No. 32),
N-[((5S)-3-{3,5-difluoro-4-[5-(3-methyl-2,3-dihydro-1H-tetrazol-5-yl)-2-furyl]phenyl}-2-oxo-1,3-oxazolidin-5-yl)methyl]acetamide (Compound No. 33),
N-({(5S)-2-oxo-3-[2,3′,6-trifluoro-4′-(1-methyl-1H-benzimidazol-2-yl)biphenyl-4-yl]-1,3-oxazolidin-5-yl}methyl)acetamide (Compound No. 34),
N-[5-(4′-{(5S)-5-[(acetylamino)methyl]-2-oxo-1,3-oxazolidin-3-yl}-2′,3,6′-trifluorobiphenyl-4-yl)-1,3,4-thiadiazol-2-yl]acetamide (Compound No. 35),
N-({(5S)-3-[2,3′-difluoro-4′-(1,3-thiazol-2-yl)biphenyl-4-yl]-2-oxo-1,3-oxazolidin-5-yl}methyl)acetamide (Compound No. 36),
N-[(3-{2,3′-difluoro-4′-[5-(hydroxymethyl)-4,5-dihydroisoxazol-3-yl]biphenyl-4-yl}-2-oxo-1,3-oxazolidin-5-yl)methyl]acetamide (Compound No. 37),
N-[((5S)-3-{3-fluoro-4-[2-(1H-imidazol-1-yl)pyrimidin-5-yl]phenyl}-2-oxo-1,3-oxazolidin-5-yl)methyl)acetamide (Compound No. 38),
N-({(5S)-2-oxo-3-[2,3′,6-trifluoro-4′-(1,3-thiazol-2-yl)biphenyl-4-yl]-1,3-oxazolidin-5-yl}methyl)acetamide (Compound No. 39),
N-[((5S)-2-oxo-3-{2,3′,6-trifluoro-4′-[(5S)-5-(hydroxymethyl)-2-oxo-1,3-oxazolidin-3-yl]biphenyl-4-yl}-1,3-oxazolidin-5-yl)methyl]acetamide (Compound No. 40),
N-({(5S)-3-[2,3′-difluoro-4′-(5-phenyl-1H-tetrazol-1-yl)biphenyl-4-yl]-2-oxo-1,3-oxazolidin-5-yl}methyl)acetamide (Compound No. 41),
N-[(3-{3-fluoro-4-[6-(5-phenyl-1H-tetrazol-1-yl)pyridin-3-yl]phenyl}-2-oxo-1,3-oxazolidin-5-yl)methyl]acetamide (Compound No. 42),
N-[((5S)-2-oxo-3-{2,3′,6-trifluoro-4′-[5-(hydroxymethyl)isoxazol-3-yl]biphenyl-4-yl}-1,3-oxazolidin-5-yl)methyl]acetamide (Compound No. 43),
N-[((5S)-3-{2,3′-difluoro-4′-[5-(hydroxymethyl)isoxazol-3-yl]biphenyl-4-yl}oxazolidin-5-yl)methyl]acetamide (Compound No. 44),
N-[((5S)-3-{3,5-difluoro-4-[6-(4-pyridin-3-yl-1H-imidazol-1-yl)pyridin-3-yl]phenyl}-2-oxo-1,3-oxazolidin-5-yl)methyl]acetamide (Compound No. 45),
N-[((5S)-3-{3,5-difluoro-4-[6-(5-phenyl-1H-tetrazol-1-yl)pyridin-3-yl]phenyl}-2-oxo-1,3-oxazolidin-5-yl)methyl]acetamide (Compound No. 46),
N-[((5S)-3-{4-[2-(1H-benzimidazol-1-yl)pyrimidin-5-yl]-3,5-difluorophenyl}-2-oxo-1,3-oxazolidin-5-yl)methyl]acetamide (Compound No. 47),
N-[((5S)-3-{4-[2-(1H-benzimidazol-1-yl)pyrimidin-5-yl]-3-fluorophenyl}-2-oxo-1,3-oxazolidin-5-yl)methyl]acetamide (Compound No. 48),
N-[((5S)-3-{3,5-difluoro-4-[2-(1H-1,2,4-triazol-1-yl)pyrimidin-5-yl]phenyl}oxazolidin-5-yl)methyl]acetamide (Compound No. 49),
N-[((5S)-3-{3-fluoro-4-[2-(4-phenyl-1H-imidazol-1-yl)pyrimidin-5-yl]phenyl}-2-oxo-1,3-oxazolidin-5-yl)methyl]acetamide (Compound No. 50),
N-[((5S)-3-{3-fluoro-4-[2-(1H-1,2,4-triazol-1-yl)pyrimidin-5-yl]phenyl}-2-oxo-1,3-oxazolidin-5-yl)methyl]acetamide (Compound No. 51),
N-({(5S)-2-oxo-3-[2,3′,6-trifluoro-4′-(5-phenyl-1H-tetrazol-1-yl)biphenyl-4-yl]-1,3-oxazolidin-5-yl}methyl)acetamide (Compound No. 52),
N-[((5S)-3-{3,5-difluoro-4-[2-(4-phenyl-1H-imidazol-1-yl)pyrimidin-5-yl]phenyl}-2-oxo-1,3-oxazolidin-5-yl)methyl]acetamide (Compound No. 53),
N-[((5S)-3-{3,5-difluoro-4-[2-(2-oxo-1,3-oxazolidin-3-yl)pyrimidin-5-yl]phenyl}-2-oxo-1,3-oxazolidin-5-yl)methyl]acetamide (Compound No. 54),
N-[((5S)-3-{3-fluoro-4-[2-(1H-1,2,3-triazol-1-yl)pyrimidin-5-yl]phenyl}-2-oxo-1,3-oxazolidin-5-yl)methyl]acetamide (Compound No. 55),
N-[((5S)-3-{4-[2-(3,5-dimethyl-1H-pyrazol-1-yl)pyrimidin-5-yl]-3-fluorophenyl}-1,3-oxazolidin-5-yl)methyl]acetamide (Compound No. 56),
N-[((5S)-3-{3-fluoro-4-[2-(2-oxo-1,3-oxazolidin-3-yl)pyrimidin-5-yl]phenyl}-2-oxo-1,3-oxazolidin-5-yl)methyl]acetamide (Compound No. 57),
N-{[(5S)-3-(4-{6-[4-(difluoromethyl)-1H-imidazol-1-yl]pyridin-3-yl}-3,5-difluorophenyl)-2-oxo-1,3-oxazolidin-5-yl]methyl}acetamide (Compound No. 58),
N-[((5S)-3-{3-fluoro-4-[2-(3-formyl-1H-pyrrol-1-yl)pyrimidin-5-yl]phenyl]-2-oxo-1,3-oxazolidin-5-yl]methyl}acetamide (Compound No. 59),
N-({(5S)-2-oxo-3-[2,3′,6-trifluoro-4′-(1H-1,2,4-triazol-1-yl)biphenyl-4-yl]-1,3-oxazolidin-5-yl}methyl)acetamide (Compound No. 60),
N-[((5S)-3-{3,5-difluoro-4-[2-(3-formyl-1H-pyrrol-1-yl)pyrimidin-5-yl]phenyl}-2-oxo-1,3-oxazolidin-5-yl)methyl]acetamide (Compound No. 61),
N-[((5S)-2-oxo-3-{2,3′,6-trifluoro-4′-[5-(hydroxymethyl)-4,5-dihydroisoxazol-3-yl]biphenyl-4-yl}-1,3-oxazolidin-5-yl)methyl]acetamide (Compound No. 62),
N-[((5S)-3-{3-fluoro-4-[2-(1H-pyrrol-1-yl)pyrimidin-5-yl]phenyl}-2-oxo-1,3-oxazolidin-5-yl)methyl]acetamide (Compound No. 63),
N-({(5S)-3-[2,3′-difluoro-4′-(1H-1,2,4-triazol-1-yl)biphenyl-4-yl]-2-oxo-1,3-oxazolidin-5-yl}methyl)acetamide (Compound No. 64),
N-({(5S)-3-[3,5-difluoro-4-(6-{4-[(E)-(methoxyimino)methyl]-1H-imidazol-1-yl}pyridin-3-yl)phenyl]-2-oxo-1,3-oxazolidin-5-yl}methyl)acetamide (Compound No. 65),
N-[((5S)-3-{3,5-difluoro-4-[6-(4-formyl-1H-imidazol-1-yl)pyridin-3-yl]phenyl}-2-oxo-1,3-oxazolidin-5-yl)methyl]acetamide (Compound No. 66),
N-[((5S)-3-{4-[6-(4-cyano-1H-imidazol-1-yl)pyridin-3-yl]-3,5-difluorophenyl}-2-oxo-1,3-oxazolidin-5-yl)methyl]acetamide (Compound No. 67),
methyl 1-[5-(4-{(5S)-5-[(acetylamino)methyl]-2-oxo-1,3-oxazolidin-3-yl}-2,6-difluorophenyl)pyridin-2-yl]-1H-imidazole-4-carboxylate (Compound No. 68),
N-({(5S)-3-[3,5-difluoro-4-(6-{4-[(E)-(hydroxyimino)methyl]-1H-imidazol-1-yl}pyridin-3-yl)phenyl]-2-oxo-1,3-oxazolidin-5-yl}methyl)acetamide (Compound No. 69),
N-({(5S)-3-[2,6-difluoro-4′-(1,3-oxazol-5-yl)biphenyl-4-yl]-2-oxo-1,3-oxazolidin-5-yl}methyl)acetamide (Compound No. 70),
N-({(5S)-3-[2,6-difluoro-4′-(5-methyl-1,3,4-oxadiazol-2-yl)biphenyl-4-yl]-2-oxo-1,3-oxazolidin-5-yl}methyl)acetamide (Compound No. 71),
N-({(5S)-3-[2,6-difluoro-4′-(2-methyl-2H-tetrazol-5-yl)biphenyl-4-yl]-2-oxo-1,3-oxazolidin-5-yl}methyl)acetamide (Compound No. 72),
N-({(5S)-3-[2,6-difluoro-4′-(1-methyl-1H-tetrazol-5-yl)biphenyl-4-yl]-2-oxo-1,3-oxazolidin-5-yl}methyl)acetamide (Compound No. 73),
N-({(5S)-2-oxo-3-[2,3′,6-trifluoro-4′-(1-methyl-1H-tetrazol-5-yl)biphenyl-4-yl]-1,3-oxazolidin-5-yl}methyl)acetamide (Compound No. 74),
N-({(5S)-3-[2,3′-difluoro-4′-(1-methyl-1H-tetrazol-5-yl)biphenyl-4-yl]-2-oxo-1,3-oxazolidin-5-yl}methyl)acetamide (Compound No. 75),
N-({(5S)-2-oxo-3-[2,3′,6-trifluoro-4′-(2-methyl-2H-tetrazol-5-yl)biphenyl-4-yl]-1,3-oxazolidin-5-yl}methyl)acetamide (Compound No. 76),
N-({(5S)-3-[2,3′-difluoro-4′-(5-methyl-1,3,4-oxadiazol-2-yl)biphenyl-4-yl]-2-oxo-1,3-oxazolidin-5-yl}methyl)acetamide (Compound No. 77),
N-({(5S)-2-oxo-3-[2,3′,6-trifluoro-4′-(5-methyl-1,3,4-oxadiazol-2-yl)biphenyl-4-yl]-1,3-oxazolidin-5-yl}methyl)acetamide (Compound No. 78),
N-[((5S)-3-{3,5-difluoro-4-[2-(1H-1,2,3-triazol-1-yl]pyrimidin-5-yl]phenyl}-2-oxo-1,3-oxazolidin-5-yl)methyl]acetamide (Compound No. 79),
N-{[(5S)-3-(3,5-difluoro-4-{6-[5-(4-fluorophenyl)-1H-tetrazol-1-yl]pyridin-3-yl}phenyl)-2-oxo-1,3-oxazolidin-5-yl]methyl}acetamide (Compound No. 80),
   Hydrochloride salt of
N-({(5S)-3-[4′-(1H-benzimidazol-2-yl)-2,3′-difluorobiphenyl-4-yl]-2-oxo-1,3-oxazolidin-5-yl}methyl)acetamide (Compound No. 81),
N-({(5S)-3-[2,3′-difluoro-4′-(2-methyl-2H-tetrazol-5-yl)biphenyl-4-yl]-2-oxo-1,3-oxazolidin-5-yl}methyl)acetamide (Compound No. 82),
N-[((5S)-3-{3,5-difluoro-4-[5-(2-methyl-2H-tetrazol-5-yl)-2-thienyl]phenyl}-2-oxo-1,3-oxazolidin-5-yl)methyl]acetamide (Compound No. 83),
N-{[(5S)-3-(3,5-difluoro-4-{6-[4-(hydroxymethyl)-1H-imidazol-1-yl]pyridin-3-yl}phenyl)-2-oxo-1,3-oxazolidin-5-yl]methyl}acetamide (Compound No. 84),
N-[((5S)-3-{3,5-difluoro-4-[2-(1H-pyrrol-1-ylpyrimidin-5-yl]phenyl}-2-oxo-1,3-oxazolidin-5-yl)methyl]acetamide (Compound No. 85),
N-[((5S)-3-[3-fluoro-4-[2-(1H-pyrazol-1-yl)pyrimidin-5-yl]phenyl]oxazolidin-5-yl)methyl]acetamide (Compound No. 86),
tert-butyl [((5R)-3-{3,5-difluoro-4-[6-(1H-1,2,4-triazol-1-yl)pyridin-3-yl]phenyl}-2-oxo-1,3-oxazolidin-5-yl)methyl]isoxazol-3-ylcarbamate (Compound No. 87),
tert-butyl [((5R)-3-{3-fluoro-4-[6-(1,3-oxazol-5-yl)pyridin-3-yl]phenyl}-2-oxo-1,3-oxazolidin-5-yl)methyl]isoxazol-3-ylcarbamate (Compound No. 88),
(5S)-3-{3,5-difluoro-4-[6-(2-methyl-2H-tetrazol-5-yl)pyridin-3-yl]phenyl}-5-[(isoxazol-3-ylamino)methyl]-1,3-oxazolidin-2-one (Compound No. 89),
(5S)-3-[2,3′-difluoro-4′-(1,3-oxazol-5-yl)biphenyl-4-yl]-5-[(isoxazol-3-ylamino)methyl]-1,3-oxazolidin-2-one (Compound No. 90),
(5S)-3-{3,5-difluoro-4-[6-(1H-imidazol-1-yl)pyridin-3-yl]phenyl}-5-[(isoxazol-3-ylamino)methyl]-1,3-oxazolidin-2-one (Compound No. 91),
(5S)-5-[(isoxazol-3-ylamino)methyl]-3-[2,3′,6-trifluoro-4′-(4-phenyl-1H-imidazol-1-yl)biphenyl-4-yl]-1,3-oxazolidin-2-one (Compound No. 92),
(5S)-3-{3,5-difluoro-4-[6-(1-methyl-1H-tetrazol-5-yl)pyridin-3-yl]phenyl}-5-[(isoxazol-3-ylamino)methyl]-1,3-oxazolidin-2-one (Compound No. 93),
N-[((5S)-3-{3,5-difluoro-4-[6-(1H-1,2,4-triazol-1-yl)pyridin-3-yl]phenyl}-2-oxo-1,3-oxazolidin-5-yl)methyl]ethanethioamide (Compound No. 94),
N-({(5S)-3-[2,3′-difluoro-4′-(1,3-oxazol-5-yl)biphenyl-4-yl]-2-oxo-1,3-oxazolidin-5-yl}methyl)ethanethioamide (Compound No. 95),
(5S)-5-(aminomethyl)-3-{3,5-difluoro-4-[6-(1H-1,2,4-triazol-1-yl)pyridin-3-yl]phenyl}-1,3-oxazolidin-2-one (Compound No. 96),
methyl [((5S)-3-{3,5-difluoro-4-[6-(1H-1,2,4-triazol-1-yl)pyridin-3-yl]phenyl}-2-oxo-1,3-oxazolidin-5-yl)methyl]carbamate (Compound No. 97),
methyl ({(5S)-3-[2,3′-difluoro-4′-(1,3-oxazol-5-yl)biphenyl-4-yl]-2-oxo-1,3-oxazolidin-5-yl}methyl)carbamate (Compound No. 98),
(5S)-3-{3,5-difluoro-4-[6-(1,3-oxazol-5-yl)pyridin-3-yl]phenyl}-(isothiocyanatomethyl)-1,3-oxazolidin-2-one (Compound No. 99),
(N-[((5S)-3-{3,5-difluoro-4-[6-(1H-1,2,4-triazol-1-yl)pyridin-3-yl]phenyl}-2-oxo-1,3-oxazolidin-5-yl)methyl]thiourea (Compound No. 100),
N-[((5S)-3-{3,5-difluoro-4-[6-(1H-1,2,4-triazol-1-yl)pyridin-3-yl]phenyl}-2-oxo-1,3-oxazolidin-5-yl)methyl]-N′-methylthiourea (Compound No. 101).

Another aspect provides for pharmaceutical compositions comprising pharmaceutically effective amounts of one or more compounds of Formula I, as described above, or pharmaceutically acceptable salts thereof and one or more pharmaceutical acceptable carriers.

Yet another aspect provides for methods of treating or preventing microbial infections comprising administering to a mammal in need thereof pharmaceutically effective amounts of one or more compounds of Formula I, as described above, or pharmaceutically acceptable salts thereof and one or more pharmaceutical acceptable carriers.

Such methods include one or more of the following embodiments. For example, the microbial infections can be caused by gram-positive and gram-negative bacteria. In another embodiment, the gram-positive bacteria can be, for example, staphylococcus spp., streptococcus spp., bacillus spp., corynebacterum spp., clostridia spp., peptostreptococus spp., listeria spp. or legionella spp.

Another aspect provides for methods of treating or preventing aerobic and anaerobic bacterial infections comprising administering to a mammal in need thereof pharmaceutically effective amounts of one or more compounds of Formula I, as described above, or pharmaceutically acceptable salts thereof and one or more pharmaceutical acceptable carriers.

Another aspect provides for processes for preparing compounds of Formula X,

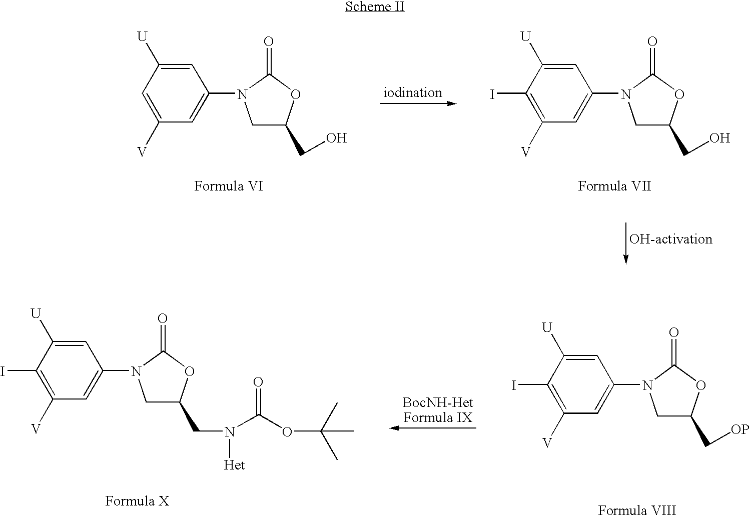

comprising the steps of:
a. reacting compounds of Formula VI with one or more iodinating agents to form compounds of Formula VII;
b. reacting compounds of Formula VII with one or more OH-protecting group reagents to form compounds of Formula VIII; and
c. reacting compounds of Formula VIII with compounds of Formula IX to form compounds of Formula X;

wherein Het can be a heterocyclyl or heteroaryl,
P is a protecting group; and
U and V can be independently selected from hydrogen (wherein both U and V cannot be H at the same time), lower (C₁₋₆) alkyl and halogen.

This process can include one or more of the following embodiments. For example, compounds of Formula VI can be reacted to form compounds of Formula VII in the presence of one or more iodinating agents, for example, iodine/silver trifluoroacetate, iodine monochloride in acetic acid or mixtures thereof.

The reaction of compounds of Formula VII to form compounds of Formula VIII can be carried out using one or more protecting group reagents, for example, methanesulfonyl chloride, toluenesulfonyl, triflic anhydride or mixtures thereof.

The reaction of compounds of Formula VIII to form compounds of Formula X can be carried out in the presence of one or more bases, for example, metal hydrides, e.g., sodium hydride, potassium hydride, lithium hydride or mixtures thereof.

Another aspect provides for processes for preparing compounds of Formulae XVIII, XIX and XIXa,

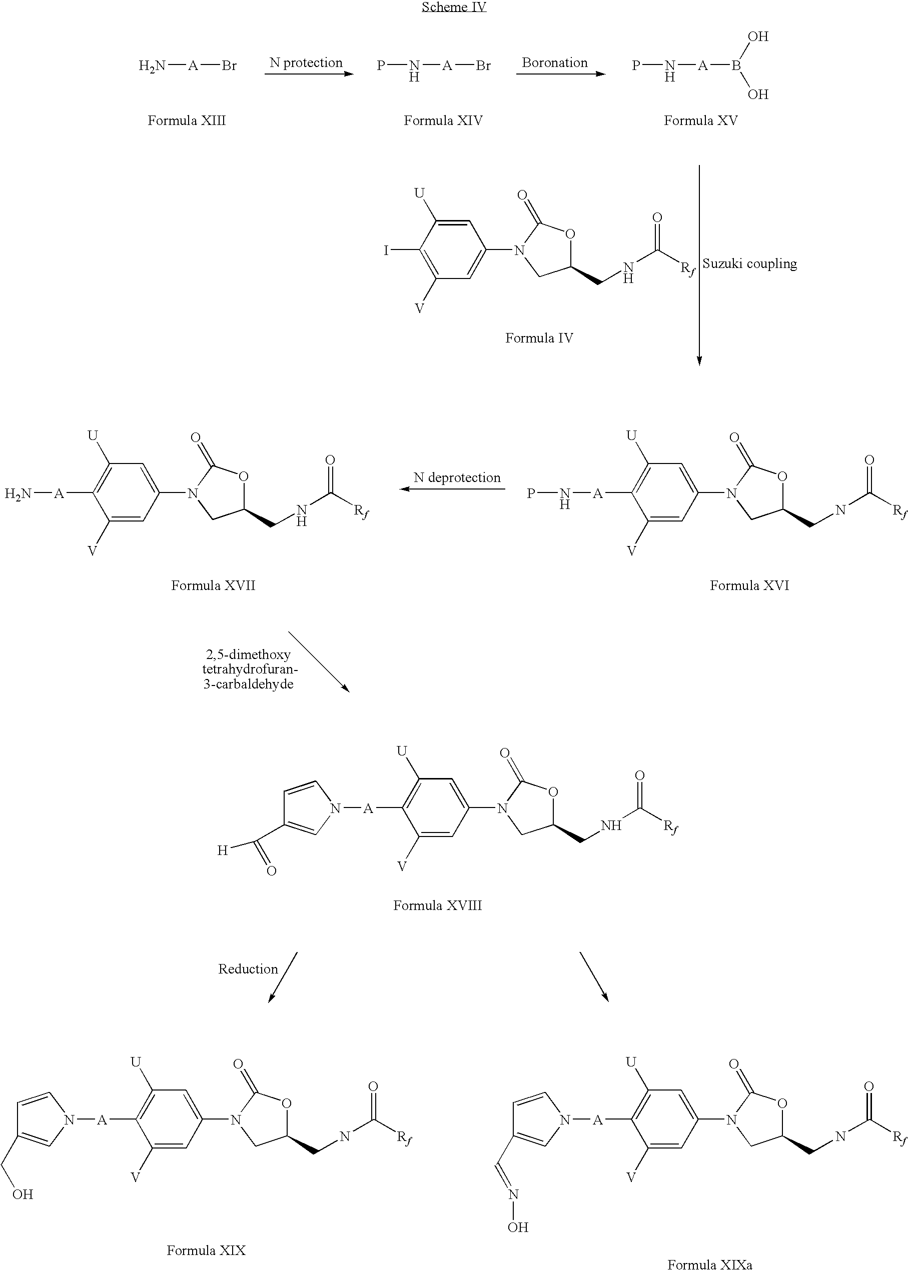

comprising the steps of:
a. reacting compounds of Formula XIII (wherein A and R_{f} are the same as described earlier) with one or more protecting group reagents to form compounds of Formula XIV;
b. reacting compounds of Formula XIV with one or more boronating agents to form compounds of Formula XV;
c. reacting compounds of Formula XV with compounds of Formula IV to form compounds of Formula XVI;
d. reacting compounds of Formula XVI with one or more deprotecting agents to form compounds of Formula XVII;
e. reacting compounds of Formula XVII with 2,5-dimethoxytetrahydrofuran-3-carbaldehyde to form compounds of Formula XVIII;
f. reducing compounds of Formula XVIII to form compounds of Formula XIX; and
g. reacting compounds of Formula XVIII with hydroxylamine hydrochloride in methanol to form compounds of Formula XIXa,

wherein A can be

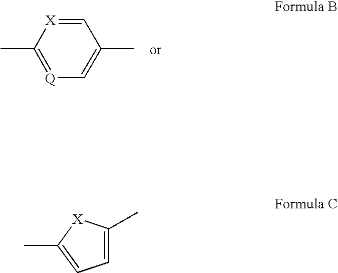

Q and X can be independently selected from —N—, —O—, —C—F, —CH— and —S—;
U and V can be independently selected from hydrogen (wherein both U and V cannot be H at the same time), lower (C₁₋₆) alkyl or halogen; and
R_{f} can be selected from hydrogen, alkyl, alkenyl, alkynyl, alkoxy, cycloalkyl, aryl, aralkyl, heteroaryl, heterocyclyl, heteroarylalkyl or heterocyclylalkyl.

The reaction of compounds of Formula XIII to form compounds of Formula XIV can be carried out in the presence of one or more organic bases, for example, nitrogen-containing base, e.g., triethylamine, 4-(dimethyl)-amino pyridine, N-methyl morpholine or mixtures thereof. Further, this reaction can be carried out with one or more protecting group reagents, for example, t-butylcarbamate (BOC), 9-fluorenylmethyl carbamate (Fmoc) or mixtures thereof.

The reaction of compounds of Formula XIV to form compounds of Formula XV can be carried out in the presence of one or more bases, for example, alkyl lithium, e.g., n-butyl lithium, sec-butyl lithium, tert-butyl lithium or mixtures thereof. In addition, this reaction can be carried out using one or more boronating agents, for example, triisopropyl borate, trimethyl borate, phenyl boronic acid, 1,4-phenylenediboronic acid, 3-methoxyphenylboronic acid or mixtures thereof.

The reaction of compounds of Formula XV to form compounds of Formula XVI can be carried out in the presence of one or more bases, for example, carbonates, e.g., sodium carbonate, potassium carbonate, cesium carbonate or mixtures thereof. This reaction also can be carried out in the presence of one or more catalysts, for example, dichlorobistriphenylphosphine palladium (II), tetrakistriphenylphosphine palladium (0) or a mixture of palladium diacetate, triphenyl phosphine or mixtures thereof.

The reaction of compounds of Formula XVI to form compounds of Formula XVII can be carried out the presence of one or more acids, for example, hydrochloric acid in ethanol, trifluoroacetic acid in dichloromethane or mixtures thereof.

The reaction of compounds of Formula XVII with 2,5-dimethoxytetrahydrofuran-3-carbaldehyde to form compounds of Formula XVIII can be carried out in the presence of one or more reagents, for example, acetic acid, acetic anhydride or mixtures thereof.

The reduction of compounds of Formula XVIII to form compounds of Formula XIX can be carried out in the presence of one or more reducing agents, for example, sodium borohydride, sodium borohydride, lithium borohydride, sodium diisopropyl aluminum hydride or mixtures thereof.

The reaction of compounds of Formula XVIII to form compounds of Formula XIX a can be carried out in the presence of one or more reducing agents, for example, NaBH4, NaBH3CN or mixtures thereof in an alcohol, e.g., methanol or ethanol.

Another aspect provides for processes for making compounds of Formulae XXIV, XXV and XXVI,

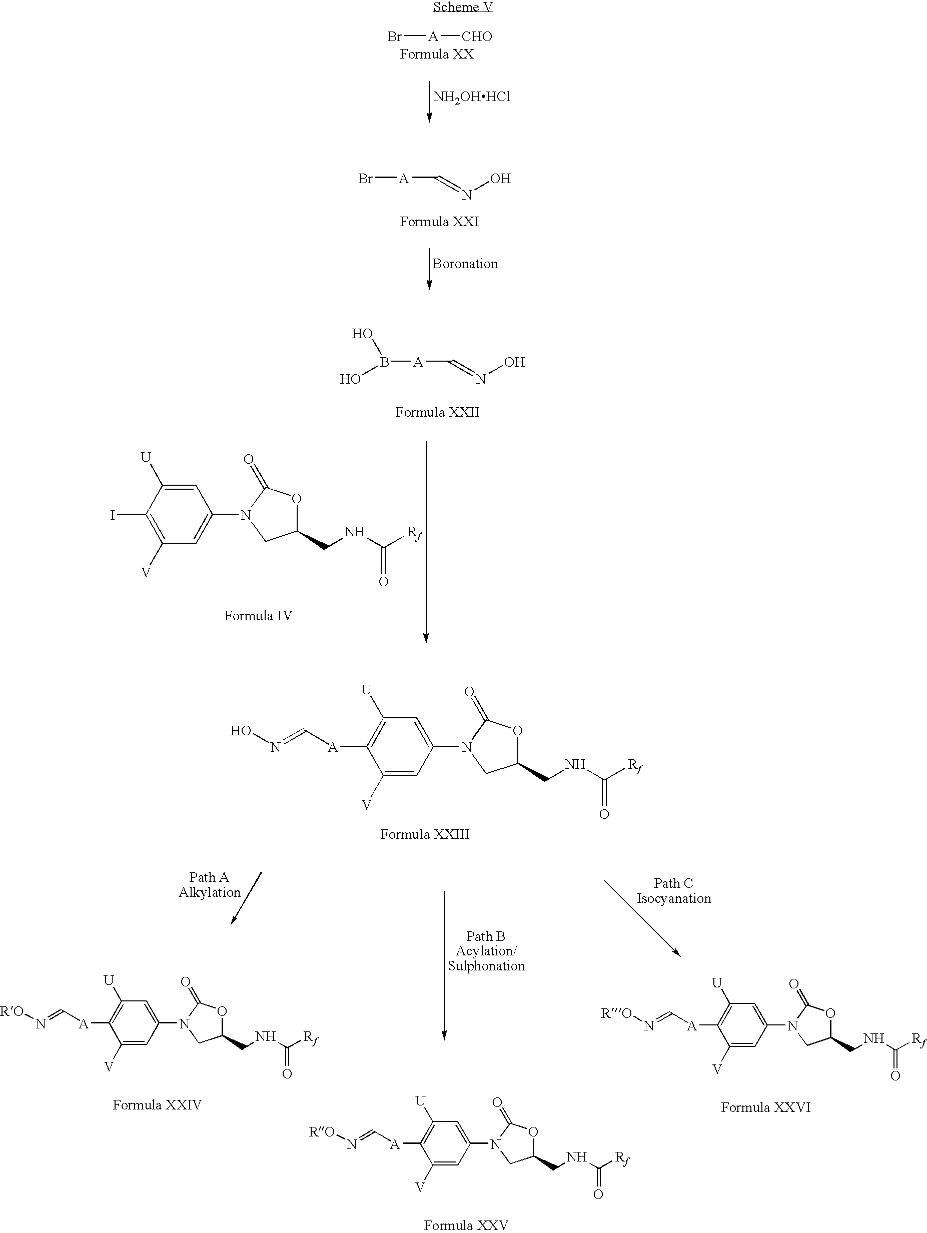

comprising the steps of:
a. reacting compounds of Formula XX with hydroxylamine hydrochloride to form compounds of Formula XXI;
b. reacting compounds of Formula XXI with one or more borating agents to form compounds of Formula XXII;
c. cross coupling compounds Formula XXII with compounds of Formula IV to form compounds of Formula XXIII; and
d. reacting compounds of Formula XXIII with one or more alkylating agents to form compounds of Formula XXIV (path A);
e. reacting compounds of Formula XXIII with one or more acylating or sulfonating agents to form compounds of Formula XXV (path B); or
f. reacting compounds of Formula XXIII with one or more isocyanating agents to form compounds of Formula XXVI, (Path C),
wherein A is

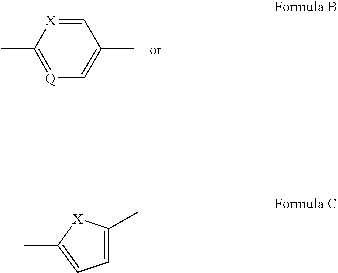

Q and X can be independently selected from —N—, —O—, —C—F, —CH— or —S—;
U and V can be independently selected from hydrogen (wherein both U and V cannot be H at the same time), lower (C₁₋₆) alkyl or halogen;
R_{f} can be hydrogen, alkyl, alkenyl, alkynyl, alkoxy, cycloalkyl, aryl, aralkyl, heteroaryl, heterocyclyl, heteroarylalkyl, or heterocyclylalkyl;
R′ can be alkyl;
R″ can be acyl or sulfonyl; and
R′″ can be isocyanate.

These processes can include one or more of the following embodiments. For example, the reaction of compounds of Formula XXI to form compounds of Formula XXII can be carried out in the presence of one or more bases, for example, alkyl lithium compounds, e.g., n-butyl lithium, sec-butyl lithium, tert-butyl lithium or mixtures thereof.

The reaction of compounds of Formula XXI to form compounds of Formula XXII can be carried out using one or more boronating agents, for example, triisopropyl borate, trimethyl borate, phenyl boronic acid, 1,4-phenylenediboronic acid, 3-methoxyphenylboronic acid or mixtures thereof.

The cross coupling reaction of compounds of Formula XXII with compounds of Formula IV to form compounds of Formula XXIII can be carried out in the presence of one or more bases, for example, carbonates, e.g., sodium carbonate, potassium carbonate, cesium carbonate or mixtures thereof. The cross coupling reaction also can be carried out in the presence of one or more catalysts, for example, dichlorobistriphenylphosphine palladium (II), tetrakistriphenylphosphine palladium (0) or mixtures thereof.

The reaction of compounds of Formula XXIII to form compounds of Formula XXIV (Path A) can be carried out in the presence of one or more bases, for example, hydroxides, e.g., potassium hydroxide, sodium hydroxide or mixtures thereof. This reaction also can be carried out using one or more alkylating agents, for example, 3,4-difluorobenzyl bromide, ethyl iodide, methyl iodide or mixtures thereof. Further, this reaction can be carried out in the presence of one or more phase transfer catalysts, for example, tetrabutylammonium iodide, tetrabutylammonium bromide, potassium iodide or mixtures thereof.

The reaction of compounds of Formula XXIII to form compounds of Formula XXV (Path B) can be carried out in the presence of one or more bases, for example, nitrogen-containing compounds, e.g., triethylamine, diisopropylamine, N-methyl morpholine or mixtures thereof. This reaction can be carried out in the presence of one or more acylating agents and/or one or more sulfonating agents, for example, benzoyl chloride, acetyl chloride, methanesulfonyl chloride or mixtures thereof.

The reaction of compounds of Formula XXIII to form compounds of Formula XXVI (Path C) can be carried out in the presence of one or more bases, for example, metal hydrides, e.g., sodium hydride, lithium hydride or mixtures thereof. This reaction also can be carried out in one or more isocyanating agents, for example, trifluoromethylphenyl isocyanate, p-fluorophenyl isocyanate, tert-butyl isocyanate or mixtures thereof.

Another aspect provides for processes for preparing compounds of Formula XXVII,

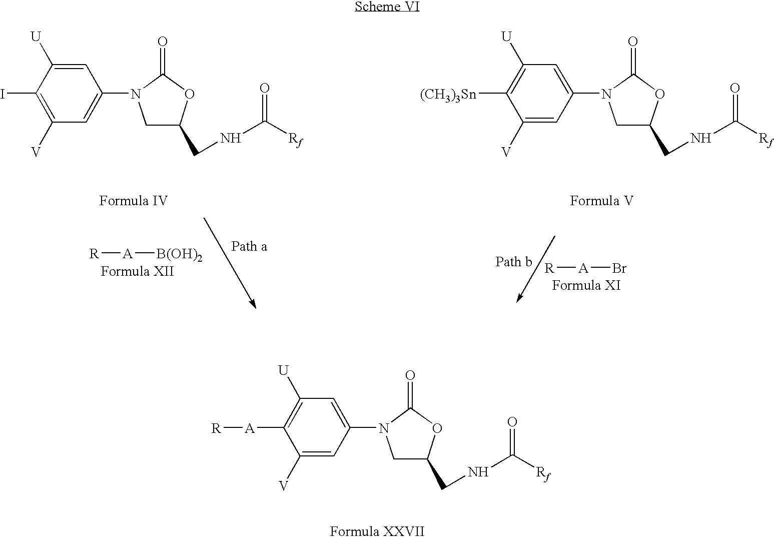

comprising reacting compounds of Formula IV with compounds of Formula XII to form compounds of Formula XXVII, (Path a); or
reacting compounds of Formula V with compounds of Formula XI to form compounds of Formula XXVII, (Path b),
wherein A can be

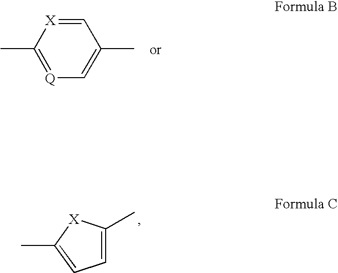

U and V can be independently selected from hydrogen (wherein both U and V cannot be H at the same time), lower (C₁₋₆) alkyl or halogen;
R_{f} can be selected from hydrogen, alkyl, alkenyl, alkynyl, alkoxy, cycloalkyl, aryl, aralkyl, heteroaryl, heterocyclyl, heteroarylalkyl or heterocyclylalkyl, and
R can be CH═NOR_{f}, CH═NOC(═O)R_{f}, CH═NOSO₂R_{f}, CH═NOC(═O)R_{f}, CH═NOSO₂R_{f}, CH═NOC(═O)NHR_{f}, heterocyclyl or heteroaryl.

These processes can include one or more of the following embodiments. The reaction of compounds of Formula XII with compounds of Formula IV to form compounds of Formula XXVII can be carried out in the presence of one or more bases, for example, carbonates, e.g., sodium carbonate, potassium carbonate, cesium carbonate or mixtures thereof. The reaction of compounds of Formula XII with compounds of Formula IV to form compounds of Formula XXVII can also be carried out the presence of one or more catalysts, for example, dichlorobistriphenylphosphine palladium (II), tetrakistriphenylphosphine palladium (0), a mixture of palladium diacetate and triphenyl phosphine, or mixtures thereof.

The reaction of compounds of Formula XI with compounds of Formula V to form compounds of Formula XXVII can be carried out in the presence of one or more bases, for example, nitrogen-containing compounds, e.g., triethylamine, 4-dimethylamino pyridine, N-methyl morpholine or mixtures thereof. This reaction also can be carried out the presence of one or more catalysts, for example, dichlorobistriphenylphosphine palladium (II), tetrakistriphenylphosphine palladium (0) or mixtures thereof.

Another aspect provides for processes for preparing compounds of Formulae XXVIII and Formula XXIX,

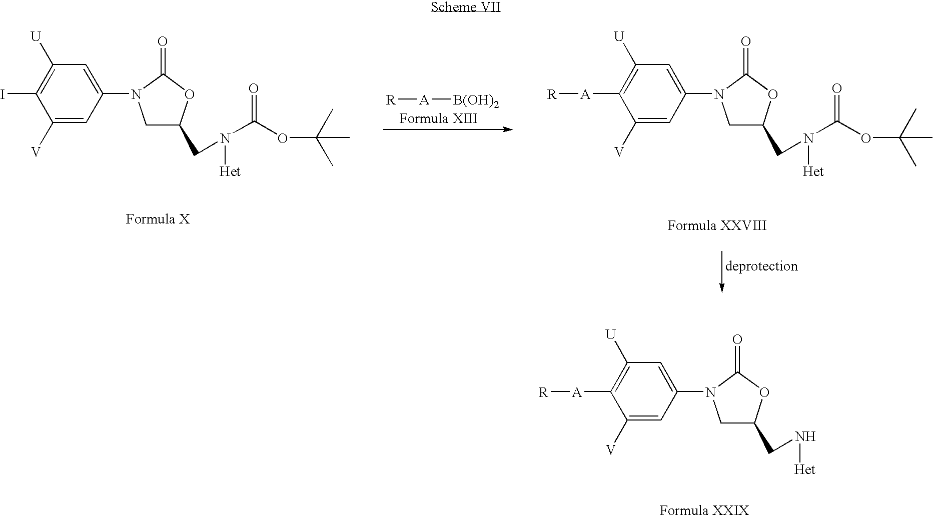

comprising the steps of:
a. reacting compounds of Formula X with compounds of Formula XIII to form compounds of Formula XXVIII; and
b. optionally reacting compounds of XXVIII with one or more deprotecting agents to form compounds of Formula XXIX,

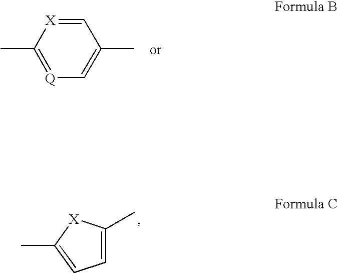

wherein A can be
Q and X can be independently selected from —N—, —O—, —C—F, —CH— or —S—,
U and V can be independently selected from hydrogen (wherein both U and V cannot be H at the same time), lower (C₁₋₆) alkyl or halogen,
R can be CH═NOR_{f}, CH═NOC(═O)R_{f}, CH═NOSO₂R_{f}, CH═NOC(═O)R_{f}, CH═NOSO₂R_{f}, CH═NOC(═O)NHR_{f}, heterocyclyl or heteroaryl, wherein
   R_{f} can be selected from hydrogen, alkyl, alkenyl, alkynyl, alkoxy, cycloalkyl, aryl aralkyl, heteroaryl, heterocyclyl, heteroarylalkyl or heterocyclylalkyl; and
Het can be heterocyclyl or heteroaryl.

The reaction of compounds of Formula X with compounds of Formula XIII to form compounds of Formula XXVIII can be carried out using one or more bases selected from sodium carbonate, potassium carbonate, cesium carbonate or mixtures thereof. This reaction also can be carried out in the presence of one or more catalysts, for example, dichlorobistriphenylphosphine palladium (II), tetrakistriphenylphosphine palladium (0), a mixture of palladium diacetate and triphenyl phosphine, or mixtures thereof.

The deprotection of compounds of Formula XXVIII to form compounds of Formula XXIX can be carried out in the presence of hydrochloric acid in ethanol or trifluoroacetic acid in dichloromethane.

Another aspect provides for processes for preparing compounds of Formulae XXX, XXXI, XXXII, XXXIII, XXXIV and XXXV,

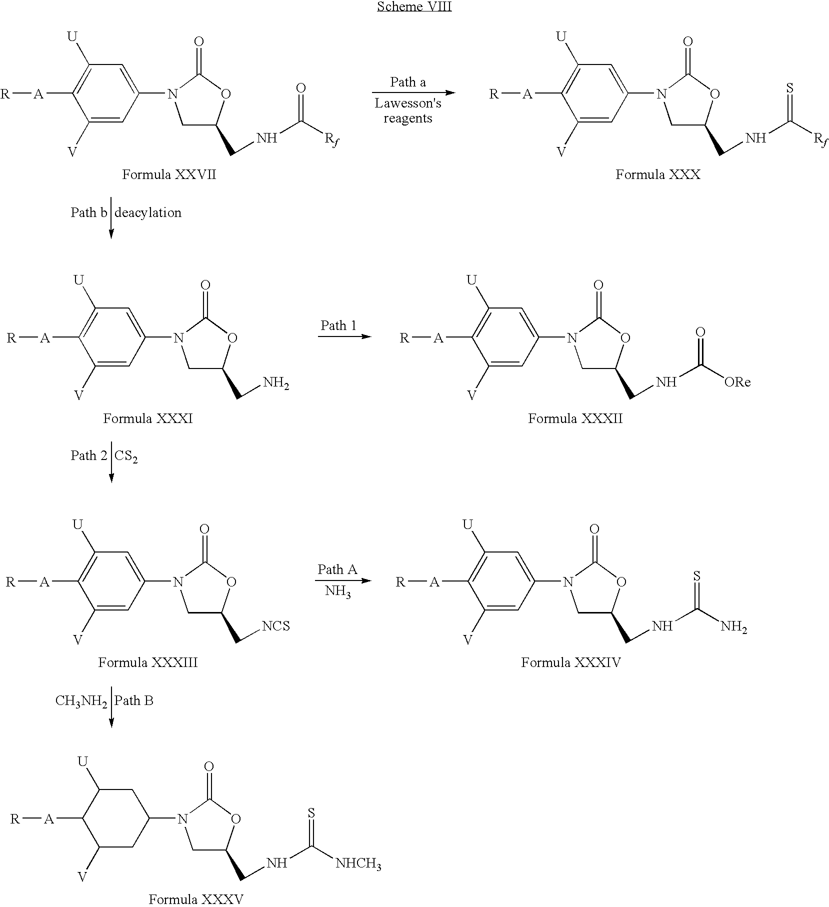

comprising the steps of:
a. reacting compounds of Formula XXVII with Lawesson's reagent to form compounds of Formula XXX, (path a); or
b. deacylating compounds of Formula XXVII to form amines of Formula XXXI, (path b);
   i. reacting compounds of Formula XXXI with alkylchloroformate to form compound of Formula XXXII (path 1); or
   ii. reacting compounds of Formula XXXI with carbon disulfite to form compounds of Formula XXXIII (path 2);
      A. reacting compounds of Formula XXXIII with methanolic ammonia to form compounds of Formula XXXIV (path A); or
      B. reacting compounds of Formula XXXIII with methylamine to yield compounds Formula XXXV (path B),

wherein A can be

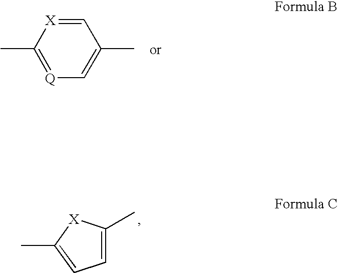

Q and X can be independently selected from —N—, —O—, —C—F, —CH— or —S—,

U and V can be independently selected from hydrogen (wherein both U and V cannot be H at the same time), lower (C₁₋₆) alkyl or halogen;

R_{f} can be selected from hydrogen, alkyl, alkenyl, alkynyl, alkoxy, cycloalkyl, aryl, aralkyl, heteroaryl, heterocyclyl, heteroarylalkyl or heterocyclylalkyl,

R can be CH═NOR_{f}, CH═NOC(═O)R_{f}, CH═NOSO₂R_{f}, CH═NOC(═O)R_{f}, CH═NOSO₂R_{f}, CH═NOC(═O)NHR_{f}, heterocyclyl or heteroaryl; and

Rₑ can be alkyl group.

Compounds of Formula XXVII can be deacylated in the presence of hydrochloride acid.

Compounds of Formula XXXI can be reacted with carbon disulfite to form compounds of Formula XXXIII in the presence of one or more bases, for example, triethylamine, 4-dimethylamino pyridine, N-methyl morpholine or mixtures thereof.

Compounds of Formula XXXIII can be reacted with methylamine to form compounds of Formula XXXV in the presence of one or more bases, for example, triethylamine, diisopropylamine, pyridine or mixtures thereof.

### DETAILED DESCRIPTION OF THE INVENTION

The present invention provides for processes for the synthesis of phenyloxazolidinones derivatives of Formula I,

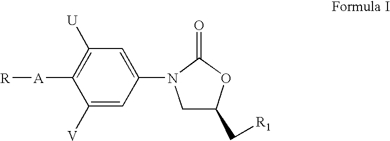

and their pharmaceutically acceptable salts, pharmaceutically acceptable solvates, esters, enantiomers, diastereomers, N-oxides, polymorphs, prodrugs or metabolites, wherein

### A can be

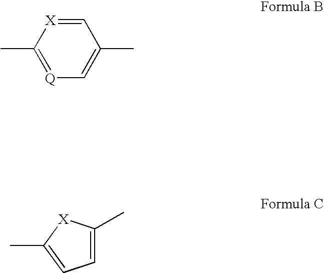

wherein Q and X can be independently selected from —N—, —O—, —C—F, —CH— or —S—;

U and V can be independently selected from hydrogen (wherein both U and V cannot be H at the same time), lower (C₁₋₆) alkyl or halogen (e.g., Cl, F or Br);
R is CH═NOR_{f}, CH═NOC(═O)R_{f}, CH═NOSO₂R_{f}, CH═NOC(═O)NHR_{f}, heterocyclyl or heteroaryl, wherein
R_{f} is hydrogen, alkyl, alkenyl, alkynyl, alkoxy, cycloalkyl, aryl, aralkyl, heteroaryl, heterocyclyl, heteroarylalkyl or heterocyclylalkyl;
   R₁ is azido, NCS, NHYR_{f}, NRⱼ C(=T)NR_{f}R_{q}, NR_{f}R_{q}, or NRⱼ(C═O)ORₛ, wherein,
Y is (C═O), (C═S) or SO₂,
R_{f} is the same as defined earlier,
T is O, S, —N(CN), —N(NO₂), or —CH(NO₂),
Rⱼ is hydrogen, alkyl, alkenyl, cycloalkyl, aryl, aralkyl, heterocyclyl, heteroaryl, heteroarylalkyl or heterocyclylalkyl,
R_{q} is hydrogen, alkyl, cycloalkyl, aryl, aralkyl, heteroaryl, heterocyclyl, heteroarylalkyl or heterocyclylalkyl, and
Rₛ is alkyl, alkenyl, alkynyl, cycloalkyl, aryl, aralkyl, heteroarylalkyl or heterocyclylalkyl;
   with the provisos that
when U is H, V is F, R₁ is NHCOCH₃ and A is Formula B (wherein Q or X is N), then R is a five-membered heteroaryl ring containing two or four N atoms (wherein the five membered heteroaryl ring containing four N atom is linked through the N-atom to Formula B and is always substituted),
when A is Formula B (wherein Q and X both are N) and U,V and R₁ are as defined above then R cannot be a five membered heterocyclyl ring containing 2 hetero atoms.

Compounds described herein can be useful antimicrobial agents, effective against a number of human and veterinary pathogens, particularly aerobic and Gram-positive bacteria, including multiply-antibiotic resistant staphylococci and streptococci, as well as anaerobic organisms, for example, Mycobacterium tuberculosis and other Mycobacterium species.

For preparing pharmaceutical compositions from the compounds described by this invention, inert, pharmaceutically acceptable carriers can be either solid or liquid. Solid form preparations include powders, tablets, dispersible granules, capsules, cachets, suppositories and ointments. A solid carrier can be one or more substances which may also act as diluents, flavoring agents, solubilizers, lubricants, suspending agents, binders, or tablets disintegrating agents; it can also be as finely divided solid which is in admixture with the finely divided active compound. For the preparation of tablets, the active compound is mixed with carrier having the necessary binding properties in suitable proportions and compacted in the shape and size desired. The powders and tablets can, in some embodiments, contain from about 5 to about 70 percent of the active ingredient. Suitable solid carriers are lactose, pectin, dextrin, starch, gelatin, tragacanth, low melting wax, cocoa butter and the like. The term “preparation” is intended to include the formulation of the active compound with encapsulating material as carrier providing a capsule in which the active component (with or without other carriers) is surrounded by carrier, which is thus in association with it. Similarly, capsules can be used, as solid dosage forms suitable for oral administration.

Liquid form preparations include solutions suspensions and emulsions. As an example may be mentioned water or water-propylene glycol solutions for parenteral injection. Such solutions are prepared so as to be acceptable to biological systems with respect to isotonicity, pH, and other parameters. Liquid preparations can also be formulated in solution in aqueous polyethylene glycol solution. Aqueous solutions suitable for oral use can be prepared by dissolving the active component in water and adding suitable colorants, flavors, stabilizing, and thickening agents as desired. Aqueous suspension suitable for oral use can be made by dispersing the finely divided active component in water with viscous material, for example, natural or synthetic gums, resins, methyl cellulose, sodium carboxymethyl cellulose and other suspending agents.

Ointment preparations can contain heavy metal salts of a compound of Formula I with a physiologically acceptable carrier. The carrier is desirably a conventional water-dispersible hydrophilic or oil-in-water carrier, particularly a conventional semi-soft or cream-like water-dispersible or water soluble, oil-in-water emulsion infected surface with a minimum of discomfort. Suitable compositions may be prepared by merely incorporating or homogeneously admixing finely divided compounds with the hydrophilic carrier or base or ointment.

The pharmaceutical preparation can be in unit dosage form. In such forms, the preparation is subdivided into unit doses containing appropriate quantities of the active component. The unit dosage form can be a packaged preparation, the package containing discrete capsules, powders in vials or ampoules and ointments capsule, cachet, tablet, gel, or cream itself or it can be the appropriate number of any of these packaged forms.

The quantity of active compound in a unit dose of preparation may be varied or adjusted from less than 1 mg to several grams according to the particular application and the potency of the active ingredient.

In therapeutic use as agents for treating bacterial infections the compounds utilized in the pharmaceutical method of this invention are administered at the initial dosage of about 3 mg to about 40 mg per kilogram daily. The dosages, however, may be varied depending upon the requirements of the patient and the compound being employed. Determination of the proper dosage for a particular situation is within the smaller dosages, which are less than the optimum dose. Small increments until the optimum effect under the daily dosage may be divided and administered in portions during the day if desired.

In one aspect, processes for the synthesis of compounds of Formula I are provided. Pharmaceutically acceptable non-toxic acid addition salts of the compounds described herein may be formed with one or more inorganic or organic acids by methods well known in the art.

The present invention also encompasses prodrugs of the compounds described herein. In general, such prodrugs can be functional derivatives of these compounds, which can readily be converted in vivo into defined compounds. Conventional procedures for selecting and preparing suitable prodrugs are known to one of ordinary skill in the art.

Also provided are pharmaceutically acceptable salts, pharmaceutically acceptable solvates, enantiomers, diastereomers, N-oxides, prodrugs, and metabolites of the compounds described herein in combination with one or more pharmaceutically acceptable carriers and optionally included excipients(s).

Other advantages will be set forth in the description which follows, and in part will be apparent from the description, or may be learned by the practice of the invention.

The following definitions apply to terms as used herein.

The term “alkyl,” unless otherwise specified, refers to a monoradical branched or unbranched saturated hydrocarbon chain having from 1 to 20 carbon atoms. This term is exemplified by groups, for example, methyl, ethyl, n-propyl, iso-propyl, n-butyl, iso-butyl, t-butyl, n-hexyl, n-decyl, tetradecyl, and the like.

Alkyl groups may further be substituted with one or more substituents selected from alkenyl, alkynyl, alkoxy, cycloalkyl, cycloalkenyl, acyl, acylamino, acyloxy, alkoxycarbonylamino, azido, cyano, halogen, hydroxy, oxo, thiocarbonyl, carboxy, carboxyalkyl, aryl, heterocyclyl, heteroaryl, arylthio, thiol, alkylthio, aryloxy, nitro, aminosulfonyl, aminocarbonylamino, —NHC(═O)R_{f}, —NR_{f}R_{q}, —C(═O)NR_{f}R_{q}, —NHC(═O)NR_{f}R_{q}, —C(═O)heteroaryl, C(═O)heterocyclyl, —O—C(═O)NR_{f}R_{q}, nitro, or —SO₂R₆ (wherein R_{f} and R_{q} are the same as defined earlier and R₆ is alkyl, alkenyl, alkynyl, cycloalkyl, aralkyl, aryl, heterocyclyl, heteroaryl, heteroarylalkyl or heterocyclylalkyl). Unless otherwise constrained by the definition, all substituents may be further substituted by 1-3 substituents chosen from alkyl, carboxy, —NR_{f}R_{q}, —C(═O)NR_{f}R_{q}, —OC(═O) NR_{f}R_{q}, —NHC(═O)NR_{f}R_{q}, hydroxy, alkoxy, halogen, CF₃, cyano, and —SO₂R₆, (wherein R₆, R_{f} and R_{q} are the same as defined earlier).

An alkyl group may also be interrupted by 1-5 atoms of groups independently chosen from oxygen, sulfur and —NRₐ—, wherein Rₐ is chosen from hydrogen, alkyl, cycloalkyl, alkenyl, cycloalkenyl, alkynyl, aryl, acyl, aralkyl, —C(═O)ORₛ (wherein Rₛ is the same as defined earlier), SO₂R₆ (wherein R₆ is as defined earlier), —C(═O)NR_{f}R_{q} (wherein R_{f} and R_{q} are as defined earlier). Unless otherwise constrained by the definition, all substituents may be further substituted by 1-3 substituents chosen from alkyl, carboxy, —NR_{f}R_{q}, —C(═O)NR_{f}R_{q}, —O—C(═O)NR_{f}R_{q} (wherein R_{f} and R_{q} are the same as defined earlier), hydroxy, alkoxy, halogen, CF₃, cyano, and —SO₂R₆ (wherein R₆ is the same as defined earlier). An alkyl group that has both substituents as defined above and is also interrupted by 1-5 atoms or groups as defined above can also be used.

The term “alkenyl,” unless otherwise specified, refers to a monoradical of a branched or unbranched unsaturated hydrocarbon group preferably having from 2 to 20 carbon atoms with cis, trans or geminal geometry. In the event that alkenyl is attached to the heteroatom, the double bond cannot be alpha to the heteroatom. Alkyl groups may further be substituted with one or more substituents selected from alkyl, alkynyl, alkoxy, cycloalkyl, cycloalkenyl, acyl, acylamino, acyloxy, —NHC(═O)R_{f}, —NR_{f}R_{q}, —C(═O)NR_{f}R_{q}, —NHC(═O)NR_{f}R_{q}, —O—C(═O)NR_{f}R_{q} (wherein R_{f} and R_{q} are the same as defined earlier), alkoxycarbonylamino, azido, cyano, halogen, hydroxy, oxo, thiocarbonyl, carboxy, arylthio, thiol, alkylthio, aryl, aralkyl, aryloxy, heterocyclyl, heteroaryl, heterocyclyl alkyl, heteroaryl alkyl, aminosulfonyl, aminocarbonylamino, alkoxyamino, nitro, SO₂R₆ (wherein R₆ is the same as defined earlier). Unless otherwise constrained by the definition, all substituents may optionally be further substituted by 1-3 substituents chosen from alkyl, carboxy, hydroxy, alkoxy, halogen, —CF₃, cyano, —NR_{f}R_{q}, —C(═O)NR_{f}R_{q}, —O—C(═O)NR_{f}R_{q} (wherein R_{f} and R_{q} are the same as defined earlier) and —SO₂R₆ (wherein R₆ is the same as defined earlier).

The term “alkynyl” unless otherwise specified refers to a monoradical of an unsaturated hydrocarbon, preferably having from 2 to 20 carbon atoms. In the event that alkynyl is attached to the heteroatom, the triple bond cannot be alpha to the heteroatom. Alkynl groups may further be substituted with one or more substituents selected from alkyl, alkenyl, alkoxy, cycloalkyl, cycloalkenyl, acyl, acylamino, acyloxy, alkoxycarbonylamino, azido, cyano, halogen, hydroxy, oxo, thiocarbonyl, carboxy, arylthio, thiol, alkylthio, aryl, aralkyl, aryloxy, aminosulfonyl, aminocarbonylamino, nitro, heterocyclyl, heteroaryl, heterocyclylalkyl, heteroarylalkyl, —NHC(═O)R_{f}, —NR_{f}R_{q}, —NHC(═O)NR_{f}R_{q}, —C(═O)NR_{f}R_{q}, —O—C(═O)NR_{f}R_{q} (wherein R_{f} and R_{q} are the same as defined earlier), —SO₂R₆ (wherein R₆ is the same as defined earlier). Unless otherwise constrained by the definition, all substituents may optionally be further substituted by 1-3 substituents chosen from alkyl, carboxy, carboxyalkyl, hydroxy, alkoxy, halogen, CF₃, —NR_{f}R_{q}, —C(═O)NR_{f}R_{q}, —NHC(═O)NR_{f}R_{q}, —C(═O)NR_{f}R_{q} (wherein R_{f} and R_{q} are the same as defined earlier), cyano, and —SO₂R₆ (wherein R₆ is the same as defined earlier).

The term “cycloalkyl” refers to cyclic alkyl groups of from 3 to 20 carbon atoms having a single cyclic ring or multiple condensed rings, for example, fused, or Spiro systems which may optionally contain one or more olefinic bonds, unless otherwise constrained by the definition. Such cycloalkyl groups include, by way of example, single ring structures, for example, cyclopropyl, cyclobutyl, cyclooctyl, cyclopentenyl, and the like, or multiple ring structures, for example, adamantanyl, and bicyclo [2.2.1] heptane, or cyclic alkyl groups to which is fused an aryl group, for example indane, and the like. Cycloalkyl groups may further be substituted with one or more substituents selected from alkyl, alkenyl, alkynyl, alkoxy, cycloalkyl, cycloalkenyl, acyl, acylamino, acyloxy, alkoxycarbonylamino, azido, cyano, halogen, hydroxy, oxo, thiocarbonyl, carboxy, carboxyalkyl, arylthio, thiol, alkylthio, aryl, aralkyl, aryloxy, aminosulfonyl, aminocarbonylamino, —NR_{f}R_{q}, —NHC(═O)NR_{f}R_{q}, —NHC(═O)R_{f}, —C(═O)NR_{f}R_{q}, —O—C (═O)NR_{f}R_{q} (wherein R_{f} and R_{q} are the same as defined earlier), nitro, heterocyclyl, heteroaryl, heterocyclylalkyl, heteroarylalkyl, SO₂—R₆ (wherein R₆ is the same as defined earlier). Unless otherwise constrained by the definition, all substituents may optionally be further substituted by 1-3 substituents chosen from alkyl, carboxy, hydroxy, alkoxy, halogen, CF₃, —NR_{f}R_{q}, —C(═O)NR_{f}R_{q}, —NHC(═O)NR_{f}R_{q}, —O—C(═O)NR_{f}R_{q} (wherein R_{f} and R_{q} are the same as defined earlier), cyano, and —SO₂R₆ (wherein R₆ is the same as defined earlier). The term “alkoxy” denotes the group O-alkyl wherein alkyl is the same as defined above. The term “aralkyl” refers to alkyl-aryl linked through alkyl (wherein alkyl is the same as defined above) portion and the said alkyl portion contains carbon atoms from 1-6 and aryl is as defined below.

The examples of aralkyl groups include benzyl and the like.

The term “aryl” herein refers to a carbocyclic aromatic group, for example phenyl, biphenyl or naphthyl ring and the like optionally substituted with 1 to 3 substituents selected from halogen (F, Cl, Br, I), hydroxy, alkyl, alkenyl, alkynyl, cycloalkyl, alkoxy, acyl, aryloxy, CF₃, cyano, nitro, COORₑ(wherein Rₑ is hydrogen, alkyl, alkenyl, cycloalkyl, aralkyl, heterocyclylalkyl, heteroarylalkyl), NHC(═O)R_{f}, —NR_{f}R_{q}, —C(═O)NR_{f}R_{q}, —NHC(═O)NR_{f}R_{q}, —O—C(═O)NR_{f}R_{q} (wherein R_{f} and R_{q} are the same as defined earlier), —SO₂R₆ (wherein R₆ is the same as defined earlier), carboxy, heterocyclyl, heteroaryl, heterocyclylalkyl, heteroarylalkyl or amino carbonyl amino. The aryl group may optionally be fused with cycloalkyl group, wherein the said cycloalkyl group may optionally contain heteroatoms selected from O, N, S.

The term “aryloxy” denotes the group O-aryl wherein aryl is the same as defined above. The term “carboxy” as defined herein refers to —C(═O)OH.

The term “heteroaryl” unless and otherwise specified refers to an aromatic ring structure containing 5 or 6 carbon atoms, or a bicyclic aromatic group having 8 to 10 carbon atoms, with one or more heteroatom(s) independently selected from N, O and S optionally substituted with 1 to 4 substituent(s) selected from halogen (F, Cl, Br, I), hydroxy, alkyl, alkenyl, alkynyl, cycloalkyl, acyl, carboxy, aryl, alkoxy, aralkyl, cyano, nitro, heterocyclyl, heteroaryl, —NR_{f}R_{q}, CH═NOH, —(CH₂)_{w}C(═O)R_{g} [wherein w is an integer from 0-4 and R_{g} is hydrogen, hydroxy, ORⱼ, NR_{f}R_{q}, —NHOR_{z} or —NHOH], —C(═O)NR_{f}R_{q} and —NHC(═O)NR_{f}R_{q}, —SO₂R₆, —O—C(═O)NR_{f}R_{q}, —O—C(═O)R_{f}, —O—C(═O)OR_{f} (wherein R₆, Rⱼ, R_{f} and R_{q} are the same as defined earlier). Unless otherwise constrained by the definition, the substituents are attached to the ring atom, be it carbon or heteroatom. Examples of heteroaryl groups include oxazolyl, imidazolyl, pyrrolyl, 1,2,3-triazolyl, 1,2,4-triazolyl, tetrazolyl, thiazolyl, oxadiazolyl, benzoimidazolyl, thiadiazolyl, pyridinyl, pyridazinyl, pyrimidinyl, thienyl, isoxazolyl, triazinyl, furanyl, benzofuranyl, indolyl, benzothiazolyl, benzoxazolyl, and the like.

The term “heterocyclyl,” unless otherwise specified refers to a non-aromatic monocyclic or bicyclic cycloalkyl group having 5 to 10 atoms in which 1 to 4 carbon atoms in a ring are replaced by heteroatoms selected from the group comprising of O, S or N, and are optionally benzofused or fused heteroaryl of 5-6 ring members and/or are optionally substituted wherein the substituents are selected from halogen (F, Cl, Br, I), hydroxy, alkyl, alkenyl, alkynyl, cycloalkyl, acyl, aryl, alkoxy, alkaryl, cyano, nitro, oxo, carboxy, heterocyclyl, heteroaryl, —O—C(═O)R_{f}, —O—C(═O)OR_{f}, —C(═O)NR_{f}R_{q}, SO₂R₆, —O—C(═O)NR_{f}R_{q}, —NHC(═O)NR_{f}R_{q}, —NR_{f}R_{q} (wherein R₆, R_{f} and R_{q} are the same as defined earlier) or guanidines. Unless otherwise constrained by the definition, the substituents are attached to the ring atom, be it carbon or heteroatom. Also unless otherwise constrained by the definition, the heterocyclyl ring may optionally contain one or more olefinic bond(s). Examples of heterocyclyl groups include oxazolidinyl, tetrahydrofuranyl, dihydrofuranyl, dihydropyridinyl, dihydroisoxazolyl, dihydrobenzofuryl, azabicyclohexyl, dihydroindolyl, piperidinyl or piperazinyl.

“Heteroarylalkyl” refers to alkyl-heteroaryl group linked through alkyl portion, wherein the alkyl and heteroaryl are the same as defined earlier. “Heterocyclylalkyl” refers to alkyl-heterocyclyl group linked through alkyl portion, wherein the alkyl and heterocyclyl are the same as defined earlier. “Acyl” refers to —C(═O)R″ wherein R″ is selected from the group hydrogen, alkyl, cycloalkyl, aryl, aralkyl, heteroaryl, heterocyclyl, heteroarylalkyl or heterocyclylalkyl.

“Substituted amino,” unless otherwise specified, refers to a group —N(Rₖ)₂ wherein each Rₖ is independently selected from hydrogen [provided that both Rₖ groups are not hydrogen (defined as “amino”)], alkyl, alkenyl, alkynyl, aralkyl, cycloalkyl, aryl, heteroaryl, heterocyclyl, heterocyclylalkyl, heteroarylalkyl, acyl, S O₂R₆ (wherein R₆ is the same as defined above), —C(=T)NR_{f}R_{q} or NHC(=T)NR_{f}R_{q} (wherein T, R_{f} and R_{q} are the same as defined earlier). Unless otherwise constrained by the definition, all substituents may optionally be further substituted by 1-3 substituents chosen from alkyl, aralkyl, cycloalkyl, aryl, heteroaryl, heterocyclyl, carboxy, carboxyalkyl, hydroxy, alkoxy, halogen, CF₃, cyano, —C(=T)NR_{f}R_{q}, —O(C═O)NR_{f}R_{q} (wherein R_{f}, R_{q} and T are the same as defined earlier) and —OC(=T)NR_{f}R_{q}, —SO₂R₆ (wherein R₆ is the same as defined earlier).

The term “leaving group” generally refers to groups that exhibit the properties of being labile under the defined synthetic conditions and also, of being easily separated from synthetic products under defined conditions. Examples of such leaving groups include, but are not limited to, halogen (F, Cl, Br, I), triflates, tosylate, mesylates, alkoxy, thioalkoxy, hydroxy radicals and the like.

The term “activated derivative of a carboxylic acid,” for example, that of a suitable protected amino acid, aliphatic acid or an aromatic acid refer to the corresponding acyl halide (e.g., acid fluoride, acid chloride and acid bromide), corresponding activated esters (e.g., nitro phenyl ester, the ester of 1-hydroxybenzotriazole or the ester of hydroxysuccinimide, HOSu) or a mixed anhydride for example anhydride with ethyl chloroformate and other conventional derivatives within the skill of the art.

The term “protecting groups” is used herein to refer to known moieties which have the property of preventing specific chemical reaction at a site on the molecule undergoing chemical modification intended to be left unaffected by the particular chemical modification. Also the term protecting group, unless otherwise specified, may be used with groups, for example, hydroxy, amino, carboxy and examples of such groups are found in T. W. Greene and P. G. M. Wuts, “Protective Groups in Organic Synthesis,” 2^{nd} Ed, John Wiley and Sons, New York, N.Y., which is incorporated herein by reference. The species of the carboxylic protecting groups, amino protecting groups or hydroxy protecting group employed are not critical as long as the derivatised moieties/moiety is/are stable to conditions of subsequent reactions and can be removed at the appropriate point without disrupting the remainder of the molecule.

The term “protecting group reagent” is used herein to refer to reagents which place protecting groups on a molecule to prevent specific chemical reaction at a site on the molecule undergoing chemical modification intended to be left unaffected by the particular chemical modification.

The term “pharmaceutically acceptable salts” refer to derivatives of the disclosed compounds of Formula I which are modified by making its acid or base salts. Examples of pharmaceutically acceptable salts include, but are not limited to, mineral or organic acids salts of basic residues, for example, amines; alkali or organic salts of acidic residues, for example, carboxylic acids; and the like.

The present invention encompasses all isotopes of atoms occurring in the present compounds. Isotopes include those atoms having the same atomic number but different mass numbers. By way of general example and without limitation, isotopes of hydrogen include tritium and deuterium. Isotopes of carbon include C-13 and C-14.

The compounds provided herein may contain one or more asymmetric carbon atoms and can thus exist as racemates, mixtures of enantiomers, single enantiomers, diastereomeric mixtures and individual diastereomers. All such isomeric forms of these compounds are expressly encompassed herein. Each stereogenic carbon may be independently of the R or S configuration. Although the specific compounds exemplified in this application may be depicted in a particular stereochemical configuration, compounds having the opposite stereochemistry at any given chiral center or mixtures thereof are encompassed herein. Although amino acids and amino acid side chains may be depicted in a particular configuration, both natural and unnatural forms are encompassed herein.

The compounds disclosed herein may be prepared by techniques well known in the art and familiar to the skilled synthetic organic chemist. In addition, the compounds of the present invention may be prepared by the following reaction sequences as depicted in Schemes I, II, III, IV, V, VI, VII and VIII. (The intermediates were prepared following the processes described in the references Eur. J. Pharm. Sci., 15, 2002, 367-378; J. Med. Chem., 2000, 43, 953-970; Indian Journal Chemistry, 1983, 22(B), 117-120; J. Med. Chem., 2003, 46, 2227-2240; J. Het. Chem., 2000, 37, 119-126; Synth. Comm., 2003, 33, 3285-3289; J. Med. Chem. 2003, 46, 284-302).

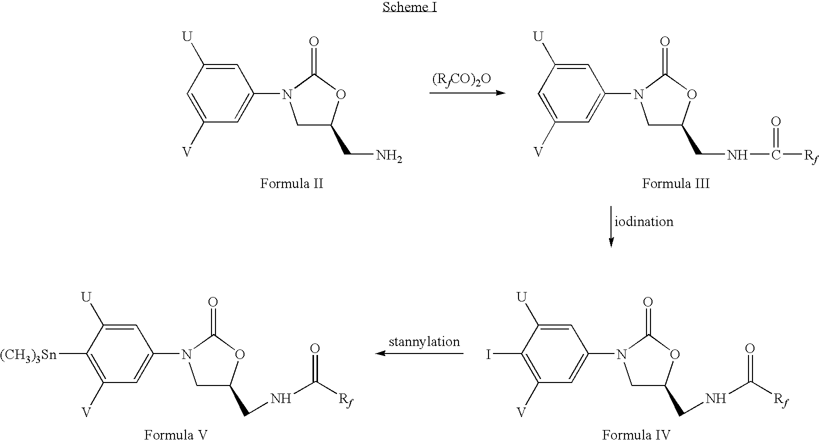

Compounds of Formula IV and Formula V can be prepared following Scheme I. Thus compounds of Formula II (wherein U and V are the same as defined earlier) can be acylated with (R_{f}CO)₂O (for example, acetic anhydride) to form compounds of Formula III. Compounds of Formula III can be iodinated to form compounds of Formula IV (wherein U, V and R_{f} is the same as defined earlier). Compounds of Formula IV can be stannylated to form compounds of Formula V.

Compounds of Formula II can be reacted with (R_{f}CO)₂O to form compounds of Formula III in one or more organic solvents, for example, dichloromethane, dichloroethane, carbon tetrachloride, tetrahydrofuran or mixtures thereof. Compounds of Formula II can be acylated with (R_{f}CO)₂O to form compounds of Formula III in the presence of one or more organic bases, for example, nitrogen-containing bases, e.g., triethylamine, diisopropylethylamine, N-methylmorpholine or mixtures thereof.

Compounds of Formula III can be iodinated to form compounds of Formula IV in one or more organic solvents, for example, chloroform, acetonitrile, carbon tetrachloride or mixtures thereof. Compounds of Formula III can also be iodinated to form compounds of Formula IV in the presence of iodine/silver trifluoroacetate, iodine monochloride in acetic acid or mixtures thereof.

Compounds of Formula IV can be stannylated with hexamethyl ditin to form compounds of Formula V in one or more organic solvents, for example, 1,4-dioxane, dimethylformamide, tetrahydrofuran or mixtures thereof. This reaction can also be carried out in the presence of one or more palladium catalysts, for example, dichlorobistriphenylphosphine palladium (II), tetrakistriphenylphosphine palladium (0) or mixtures thereof.

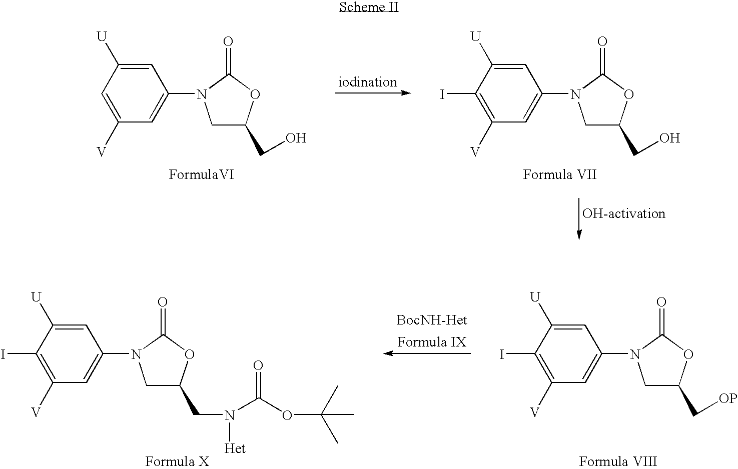

Compounds of Formula X can be prepared following Scheme II. Thus, compounds of Formula VI can be iodinated to form compounds of Formula VII (wherein U and V are the same as defined earlier). Compounds of Formula VII can be OH-activated to form compounds of Formula VIII (wherein P can be mesyl, tosyl or triflyl). Compounds of Formula VIII can be reacted with compounds of Formula IX (wherein Het can be a heterocyclyl or heteroaryl) to form compounds of Formula X.

Compounds of Formula VI can be iodinated to form compounds of Formula VII in one or more organic solvents, for example, chloroform, acetonitrile, carbon tetrachloride or mixtures thereof. This reaction can also be carried out in the presence of iodine/silver trifluoroacetate, iodine monochloride in acetic acid or mixtures thereof.

Compounds of Formula VII can be OH-activated to form compounds of Formula VIII in one or more organic solvents, for example, dichloromethane, dichloroethane, chloroform, carbon tetrachloride or mixtures thereof. This reaction can also be carried out in the presence of one or more reagents, for example, methanesulfonyl chloride, toluenesulfonyl chloride, triflic anhydride or mixtures thereof.

Compounds of Formula VIII can be reacted with compounds of Formula IX to form compounds of Formula X in one or more organic solvents, for example, dimethylformamide, tetrahydrofuran, diethyl ether, dioxane or mixtures thereof. This reaction can also be carried out in the presence of one or more bases, for example, metal hydrides, e.g., sodium hydride, potassium hydride, lithium hydride or mixtures thereof.

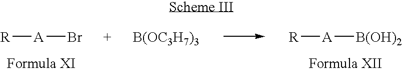

Compounds of Formula XII can be prepared following Scheme III. Thus, compounds of XI (wherein R and A are the same as described earlier) can be reacted with triisopropylborate to form compounds of Formula XII.

Alternatively, other boronating agents can be used, for example, triisopropyl borate, trimethyl borate, triethyl borate, phenyl boronic acid, 1,4-phenylenediboronic acid or 3-methoxyphenylboronic acids in place of or in addition to triisopropylborate in this reaction.

Compounds of Formula XI can be reacted with triisopropylborate to form compounds of Formula XII in one or more organic solvents, for example, tetrahydrofuran, dimethylformamide, carbon tetrachloride or mixtures thereof. This reaction can also be carried out in the presence of one or more bases, for example, alkyl lithium compounds, e.g., n-butyl lithium, sec-butyl lithium, tert-butyl lithium or mixtures thereof.

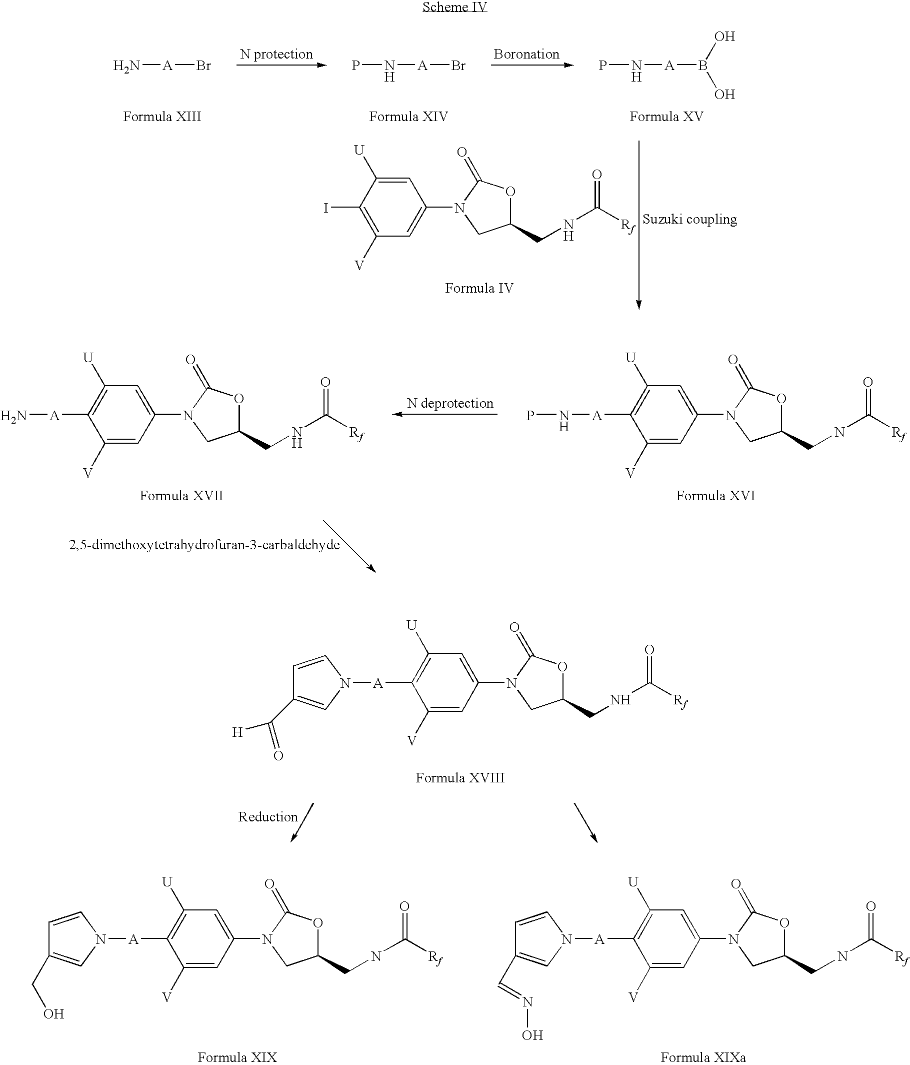

Compounds of Formula XVIII, Formula XIX and Formula XIXa can be prepared following Scheme IV. Thus, compounds of Formula XIII (wherein A is the same as described earlier) can be N-protected to form compounds of Formula XIV (wherein P can be a protecting group). Compounds of Formula XIV can be boronated to form compounds of Formula XV. Compounds of Formula XV can be cross-coupled with compounds of Formula IV (wherein U, V and R_{f} are the same as defined earlier) to form compounds of Formula XVI. Compounds of Formula XVI can be deprotected to form compounds of Formula XVII. Compounds of Formula XVII can be reacted with 2,5-dimethoxytetrahydrofuran-3-carbaldehyde to form compounds of Formula XVIII. Compounds of Formula XVIII can be reduced to form compounds of Formula XIX. Alternatively, compounds of Formula XVIII can be reacted with hydroxylamine hydrochloride to form compounds of Formula XIXa.

Compounds of Formula XIII can be N-protected to form compounds of Formula XIV with a suitable protecting group (for example, t-butylcarbamate (BOC), or 9-fluorenylmethyl carbamate (Fmoc)) and in the presence of one or more organic bases, for example, nitrogen-containing compounds, e.g., triethylamine, 4-dimethylaminopyridine or N-methyl morpholine. This reaction can also be carried out in one or more organic solvents, for example, dichloromethane, dichloroethane, carbon tetrachloride or mixtures thereof.

Compounds of Formula XIV can be boronated to form compounds of Formula XV using one or more suitable boronating agents (for example, triisopropyl borate, trimethyl borate, phenyl boronic acid, 1,4-phenylenediboronic acid, 3-methoxyphenylboronic acid or mixtures thereof) and in presence of one or more organic bases, for example, alkyl lithium compounds, e.g., n-butyl lithium, sec-butyl lithium, tert-butyl lithium or mixtures thereof. This reaction can also be carried out in one or more organic solvents, for example, tetrahydrofuran, dimethylformamide, dioxane, diethylether or mixtures thereof.

Compounds of Formula XV can be cross coupled with compounds of Formula IV to form compounds of Formula XVII in one or more organic solvents, for example, n-propanol, 1,4-dioxane or acetone. This reaction can also be carried out using one or more bases, for example, carbonates, e.g., sodium carbonate, potassium carbonate, or cesium carbonate and in the presence of one or more catalysts, for example dichlorobistriphenylphosphine palladium (II), tetrakistriphenylphosphine palladium (0), a mixture of palladium diacetate and triphenyl phosphine or mixtures thereof.

Compounds of Formula XVI can be deprotected to form compounds of Formula XVII in the presence of one or more acids, for example, hydrochloric acid in ethanol, or trifluoroacetic acid in dichloromethane.

The reaction of compounds of Formula XVII with 2,5-dimethoxytetrahydrofuran-3-carbaldehyde to form compounds of Formula XVIII can be carried out in the presence of one or more reagents, for example, acetic acid or acetic anhydride.

Compounds of Formula XVIII can be reduced to form compounds of Formula XIX in presence of one or more reducing agents, for example, sodium borohydride, lithium borohydride, diisopropyl aluminum hydride or mixtures thereof. This reaction can also be carried out in one or more organic solvents, for example, dichloromethane, methanol, ethanol or mixtures thereof.

Exemplary compounds prepared following Scheme IV include, for example:
N-[((5S)-3-{3,5-difluoro-4-[6-(3-formyl-1H-pyrrol-1-yl)pyridin-3-yl]phenyl}-2-oxo-1,3-oxazolidin-5-yl)methyl]acetamide (Compound No. 1)
N-{[(5S)-3-(3,5-difluoro-4-{6-[3-(hydroxymethyl)-1H-pyrrol-1-yl]pyridin-3-yl}phenyl)-2-oxo-1,3-oxazolidin-5-yl]methyl}acetamide (Compound No. 2)
N-({(5S)-3-[3-fluoro-4-(2-{3-[(E)-(hydroxyimino)methyl]-1H-pyrrol-1-yl}pyrimidin-5-yl)phenyl]-2-oxo-1,3-oxazolidin-5-yl}methyl)acetamide (Compound No. 3)
N-({(5S)-3-[3,5-difluoro-4-(2-{3-[(E)-(hydroxyimino)methyl]-1H-pyrrol-1-yl}pyrimidin-5-yl)phenyl]-2-oxo-1,3-oxazolidin-5-yl}methyl)acetamide (Compound No. 4)
N-{[(5S)-3-(2,3′-difluoro-4′-{3-[(E)-(hydroxyimino)methyl]-1H-pyrrol-1-yl}biphenyl-4-yl)-2-oxo-1,3-oxazolidin-5-yl]methyl}acetamide (Compound No. 24),

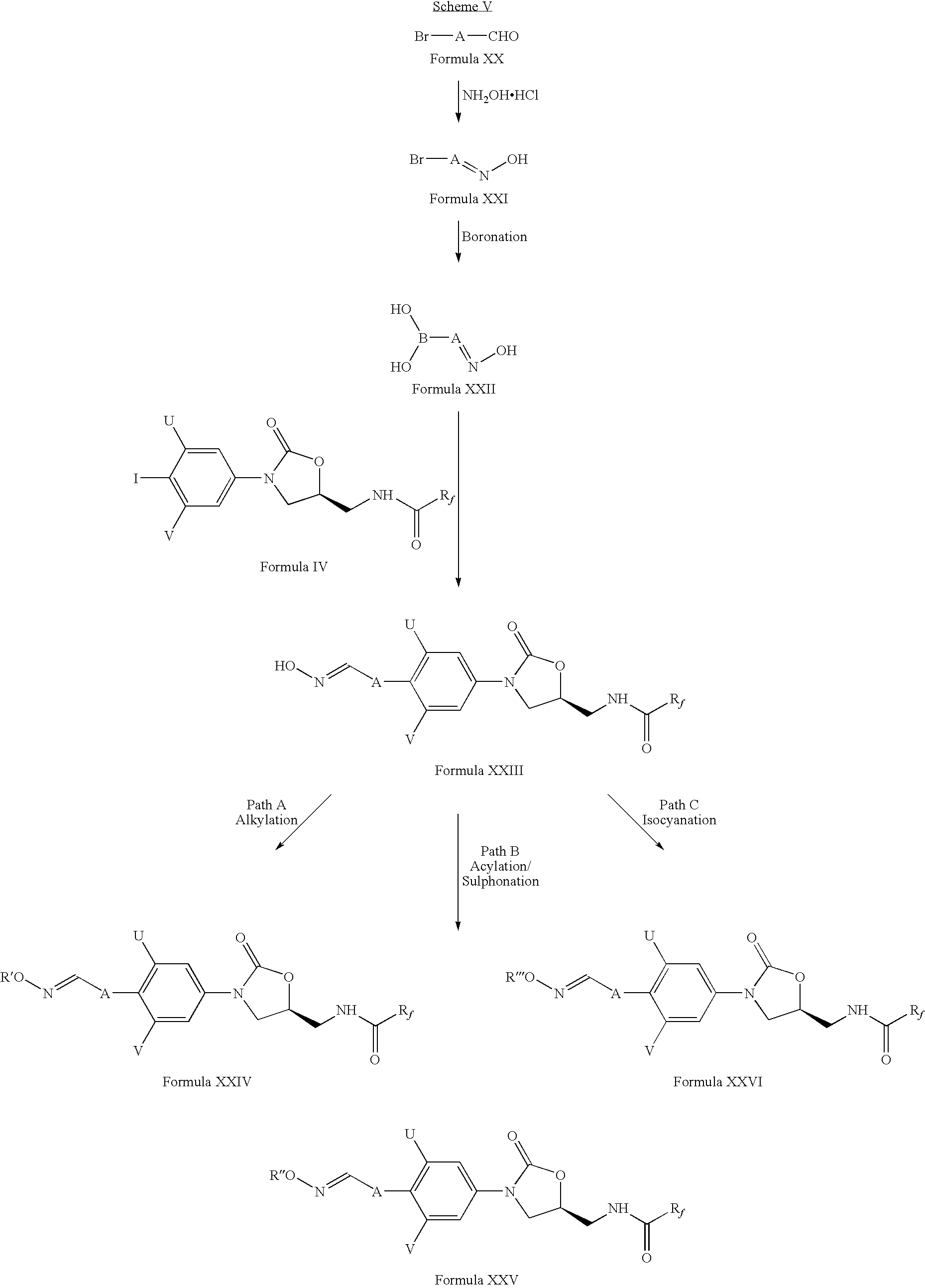

Compounds of Formula XXIV, Formula XXV, and Formula XXVI can be prepared following the Scheme V. Thus compounds of Formula XX can be reacted with hydroxylamine hydrochloride to form compounds of Formula XXI. Compounds of Formula XXI can be boronated to form compounds of Formula XXII. Compounds of Formula XXII can be cross coupled with compounds of Formula IV to form compounds of Formula XXIII.

Path A—Compounds of Formula XXIII can be alkylated to form compounds of Formula XXIV (wherein R′ can be alkyl).

Path B—Compounds of Formula XXIII can be acylated or sulfonated to form compounds of Formula XXV (wherein R″ can be acyl or sulfonyl).

Path C—Compounds of Formula XXIII can be isocyanated to form compounds of Formula. XXVI (wherein R′″ can be isocyanate).

Compounds of Formula XX can be reacted with hydroxylamine hydrochloride to form compounds of Formula XXI in one or more organic solvents, for example, ethanol, methanol, propanol or mixtures thereof.

Compounds of Formula XXI can be boronated to form compounds of Formula XXII in the presence of one or more boronating agents, for example, triisopropyl borate, trimethyl borate, triethyl borate, phenyl boronic acid, 1,4-phenylenediboronic acid, 3-methoxyphenylboronic acid or mixtures thereof. This reaction can also be carried out in presence of one or more organic bases, for example, alkyl lithium compounds, e.g., n-butyl lithium, sec-butyl lithium, tert-butyl lithium or mixtures thereof. This reaction can also be carried out in one or more organic solvents, for example, tetrahydrofuran, dimethylformamide, dioxane, diethylether or mixtures thereof.

Compounds of Formula XXII can be cross coupled with compounds of Formula IV to form compounds of Formula XXIII using one or more bases, for example, carbonates, e.g., sodium carbonate, potassium carbonate, cesium carbonate or mixtures thereof. This reaction can also be carried out in the presence of one or more catalysts, for example, dichlorobistriphenylphosphine palladium (II), tetrakistriphenylphosphine palladium (0), a mixture of palladium diacetate and triphenyl phosphine or mixtures thereof. This reaction can also be carried out in one or more organic solvents, for example, n-propanol, 1,4-dioxane, acetone or mixtures thereof.

Compounds of Formula XXIII can be alkylated to form compounds of Formula XXIV (path A) using one or more bases, for example, hydroxy bases, e.g., potassium hydroxide, sodium hydroxide or mixtures thereof and in the presence of one or more alkylating agents, for example, 3,4-difluorobenzyl bromide, ethyl iodide, methyl iodide or mixtures thereof. This reaction can also be carried out in the presence of one or more phase transfer catalysts, for example, tetrabutylammonium iodide, tetrabutylammonium bromide, potassium iodide or mixtures thereof. This reaction can also be carried out in one or more organic solvents, for example, tetrahydrofuran, dimethylformamide, dioxane, diethylether or mixtures thereof.

Compounds of Formula XXIII can be acylated to form compounds of Formula XXV (Path B) using one or more bases, for example, nitrogen-containing compounds, e.g., triethylamine, diisopropylamine, N-methyl morpholine or mixtures thereof, and in the presence of one or more acylating agents, for example, methanesulfonyl chloride, benzoyl chloride, acetyl chloride or mixtures thereof. This reaction can also be carried out in one or more organic solvents, for example, dichloromethane, toluene, dichloroethane or mixtures thereof.

Compounds of Formula XXIII can be isocyanated to form compounds of Formula XXVI (Path C) using one or more bases, for example, hydrides, e.g., sodium hydride, lithium hydride or mixtures thereof, and in the presence of one or more isocyanates, for example, trifluoromethylphenyl isocyanate, p-fluorophenyl isocyanate or mixtures thereof. This reaction can also be carried out in one or more organic solvents, for example, dichloromethane, toluene, dichloroethane or mixtures thereof.

Compounds prepared following Scheme V include, for example:
N-({(5S)-3-[4′-((E)-{[(3,4-difluorobenzyl)oxy]imino}methyl)-2,3′,6-trifluorobiphenyl-4-yl]-2-oxo-1,3-oxazolidin-5-yl}methyl)acetamide (Compound No. 5)
N-{[(5S)-3-(4′-{(E)-[(acetyloxy)imino]methyl}-2,3′,6-trifluorobiphenyl-4-yl)-2-oxo-1,3-oxazolidin-5-yl]methyl}acetamide (Compound No. 6)
N-{[(5S)-3-(4′-{(E)-[(benzoyloxy)imino]methyl}-2,3′,6-trifluorobiphenyl-4-yl)-2-oxo-1,3-oxazolidin-5-yl]methyl}acetamide (Compound No. 7)
N-({(5S)-2-oxo-3-[2,3′,6-trifluoro-4′-((E)-{[(methylsulfonyl)oxy]imino}methyl)biphenyl-4-yl]-1,3-oxazolidin-5-yl}methyl)acetamide (Compound No. 8)
N-({(5S)-2-oxo-3-[2,3′,6-trifluoro-4′-((E)-{[({[4-(trifluoromethyl)phenyl]amino}carbonyl)oxy]imino}methyl)biphenyl-4-yl]-1,3-oxazolidin-5-yl}methyl)acetamide (Compound No. 9)
N-[((5S)-3-{4′-[(E)-({[(tert-butylamino)carbonyl]oxy}imino)methyl]-2,3′,6-trifluorobiphenyl-4-yl}-2-oxo-1,3-oxazolidin-5-yl)methyl]acetamide (Compound No. 10)
N-{[(5S)-2-oxo-3-(2,3′,6-trifluoro-4′-{(E)-[({[4-fluorophenyl)amino]carbonyl}oxy) imino]methyl}biphenyl-4-yl)-1,3-oxazolidin-5-yl]methyl}acetamide (Compound No. 11)

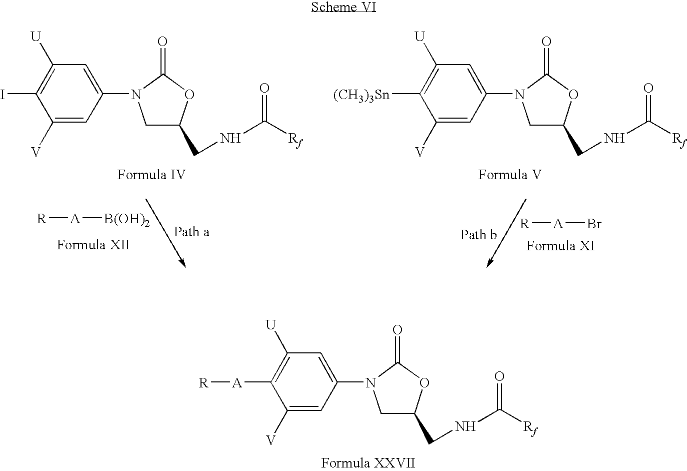

Compounds of Formula XXVII can be prepared in two ways following Scheme VI. Thus,

Path a) compounds of Formula IV (from Scheme I) can be reacted with compounds of Formula XII (from Scheme III) to form compounds of Formula XXVII; or

Path b) compounds of Formula V (from Scheme I) can be reacted with compounds of Formula XI to form compounds of Formula XXVII.

Compounds of Formula IV can be cross coupled with compounds of Formula XII to form compounds of Formula XXVII using one or more bases, for example, carbonates, e.g., sodium carbonate, potassium carbonate, cesium carbonate or mixtures thereof, and in the presence of one or more catalysts, for example, dichlorobistriphenylphosphine palladium (II), tetrakistriphenylphosphine palladium (0), a mixture of palladium diacetate and triphenyl phosphine, or mixtures thereof. This reaction can also be carried out in one or more organic solvents, for example, n-propanol, 1,4-dioxane, acetone or mixtures thereof.

Compounds of Formula V can be cross coupled with compounds of Formula XI to form compounds of Formula XXVII using one or more bases, for example, nitrogen-containing bases, e.g., triethylamine, 4-dimethylamino pyridine, N-methyl morpholine or mixtures thereof, and in the presence of one or more catalysts, for example, dichlorobistriphenylphosphine palladium (II), tetrakistriphenylphosphine palladium (0), a mixture of palladium diacetate and triphenyl phosphine or mixtures thereof. This reaction can also be carried out in one or more organic solvents, for example, dimethyl formamide, 1,4-dioxane, tetrahydrofuran or mixtures thereof. When R is an aldehyde group, it can be converted into corresponding oxime by methods known to one of ordinary skill in the art.

Compounds prepared following Scheme VI include, for example:
N-({(5S)-2-oxo-3-[2,3′,6-trifluoro-4′-(1,3-oxazol-5-yl)biphenyl-4-yl]-1,3-oxazolidin-5-yl}methyl)acetamide (Compound No. 12),
N-[((5S)-3-{3,5-difluoro-4-[6-(1H-1,2,4-triazol-1-yl)pyridin-3-yl]phenyl}-2-oxo-1,3-oxazolidin-5-yl)methyl]acetamide (Compound No. 13),
N-{[(5S)-3-(3,5-difluoro-4-{6-[5-(1,3-oxazol-4-yl)-2-furyl]pyridin-3-yl}phenyl)-2-oxo-1,3-oxazolidin-5-yl]methyl}acetamide (Compound No. 14),
N-{[(5S)-3-(3,5-difluoro-4-{6-[5-(1,3-oxazol-4-yl)-2-thienyl]pyridin-3-yl}phenyl)-2-oxo-1,3-oxazolidin-5-yl]methyl}acetamide (Compound No. 15),
N-{[(5S)-3-(3,5-difluoro-4-{6-[3-(hydroxymethyl)-1H-pyrrol-1-yl]pyridin-3-yl}phenyl)-2-oxo-1,3-oxazolidin-5-yl]methyl}acetamide (Compound No. 16),
N-({(5S)-3-[3,5-difluoro-4-(6-{3-[(E)-(hydroxyimino)methyl]-1H-pyrrol-1-yl}pyridin-3-yl)phenyl]-2-oxo-1,3-oxazolidin-5-yl}methyl) (Compound No. 17),
N-({(5S)-3-[2,3′-difluoro-4′-(5-methyl-1,2,4-oxadiazol-3-yl)biphenyl-4-yl]-2-oxo-1,3-oxazolidin-5-yl}methyl)acetamide (Compound No. 18),
N-({(5S)-2-oxo-3-[2,3′,6-trifluoro-4′-(5-methyl-1,2,4-oxadiazol-3-yl)biphenyl-4-yl]-1,3-oxazolidin-5-yl}methyl)acetamide (Compound No. 19),
N-[((5S)-3-{3,5-difluoro-4-[6-(2-methyl-2H-tetrazol-5-yl)pyridin-3-yl]phenyl}-2-oxo-1,3-oxazolidin-5-yl)methyl]acetamide (Compound No. 20),
N-[((5S)-3-{3,5-difluoro-4-[6-(1H-imidazol-1-yl)pyridin-3-yl]phenyl}-2-oxo-1,3-oxazolidin-5-yl)methyl]acetamide (Compound No. 21),
N-[((5S)-3-{3,5-difluoro-4-[6-(1-methyl-1H-tetrazol-5-yl)pyridin-3-yl]phenyl}-2-oxo-1,3-oxazolidin-5-yl)methyl]acetamide (Compound No. 22),
(5S)-3-{3,5-difluoro-4-[6-(2-methyl-2H-tetrazol-5-yl)pyridin-3-yl]phenyl}-5-[(1,3-thiazol-2-ylamino)methyl]-1,3-oxazolidin-2-one (Compound No. 23),
N-({(5S)-3-[2,3′-difluoro-4′-(5-methyl-1H-tetrazol-1-yl)biphenyl-4-yl]-2-oxo-1,3-oxazolidin-5-yl}methyl)acetamide (Compound No. 25),
N-((S)-3-{3,5-Difluoro-4-[6-[5-methyl-[1,3,4]oxadiazol-2-yl)-pyridin-3-yl]-phenyl}-2-oxo-oxazolidin-5-ylmethyl)-acetamide (Compound No. 26),
N-((S)-3-{4-[6-(5-Amino-[1,3,4]thiadiazol-2-yl)-pyridin-3-yl]-3,5-difluoro-phenyl}-2-oxo-oxazolidin-5-ylmethyl)-acetamide (Compound No. 27),
N-({(5S)-3-[4′-(1H-benzimidazol-2-yl)-2,3′,6-trifluorobiphenyl-4-yl]-2-oxo-1,3-oxazolidin-5-yl}methyl)acetamide (Compound No. 28),
N-[((5S)-3-{3,5-difluoro-4-[6-(1H-1,2,3-triazol-1-yl)pyridin-3-yl]phenyl}-2-oxo-1,3-oxazolidin-5-yl)methyl]acetamide (Compound No. 29),
N-[((5S)-3-{3,5-difluoro-4-[6-(4-phenyl-1H-imidazol-1-yl)pyridin-3-yl]phenyl}-2-oxo-1,3-oxazolidin-5-yl)methyl]acetamide (Compound No. 30),
N-[((5S)-3-{3-fluoro-4-[5-(2-methyl-2H-tetrazol-5-yl)-2-furyl]phenyl}-2-oxo-1,3-oxazolidin-5-yl)methyl]acetamide (Compound No. 31),
N-[((5S)-3-{3-fluoro-4-[5-(1-methyl-1H-tetrazol-5-yl)-2-furyl]phenyl}-2-oxo-1,3-oxazolidin-5-yl)methyl]acetamide (Compound No. 32),
N-[((5S)-3-{3,5-difluoro-4-[5-(3-methyl-2,3-dihydro-1H-tetrazol-5-yl)-2-furyl]phenyl}-2-oxo-1,3-oxazolidin-5-yl)methyl]acetamide (Compound No. 33),
N-({(5S)-2-oxo-3-[2,3′,6-trifluoro-4′-(1-methyl-1H-benzimidazol-2-yl)biphenyl-4-yl]-1,3-oxazolidin-5-yl}methyl)acetamide (Compound No. 34),
N-[5-(4′-{(5S)-5-[(acetylamino)methyl]-2-oxo-1,3-oxazolidin-3-yl}-2′,3,6′-trifluorobiphenyl-4-yl)-1,3,4-thiadiazol-2-yl]acetamide (Compound No. 35),
N-({(5S)-3-[2,3′-difluoro-4′-(1,3-thiazol-2-yl)biphenyl-4-yl]-2-oxo-1,3-oxazolidin-5-yl}methyl)acetamide (Compound No. 36),
N-[(3-{2,3′-difluoro-4′-[5-(hydroxymethyl)-4,5-dihydroisoxazol-3-yl]biphenyl-4-yl}-2-oxo-1,3-oxazolidin-5-yl)methyl]acetamide (Compound No. 37),
N-[((5S)-3-{3-fluoro-4-[2-(1H-imidazol-1-yl)pyrimidin-5-yl]phenyl}-2-oxo-1,3-oxazolidin-5-yl)methyl]acetamide (Compound No. 38),
N-({(5S)-2-oxo-3-[2,3′,6-trifluoro-4′-(1,3-thiazol-2-yl)biphenyl-4-yl]-1,3-oxazolidin-5-yl}methyl)acetamide (Compound No. 39),
N-[((5S)-2-oxo-3-{2,3′,6-trifluoro-4′-[(5S)-5-(hydroxymethyl)-2-oxo-1,3-oxazolidin-3-yl]biphenyl-4-yl}-1,3-oxazolidin-5-yl)methyl]acetamide (Compound No. 40),
N-({(5S)-3-[2,3′-difluoro-4′-(5-phenyl-1H-tetrazol-1-yl)biphenyl-4-yl]-2-oxo-1,3-oxazolidin-5-yl}methyl)acetamide (Compound No. 41),
N-[(3-{3-fluoro-4-[6-(5-phenyl-1H-tetrazol-1-yl)pyridin-3-yl]phenyl}-2-oxo-1,3-oxazolidin-5-yl)methyl]acetamide (Compound No. 42),
N-[((5S)-2-oxo-3-{2,3′,6-trifluoro-4′-[5-(hydroxymethyl)isoxazol-3-yl]biphenyl-4-yl}-1,3-oxazolidin-5-yl)methyl]acetamide (Compound No. 43),
N-[((5S)-3-{2,3′-difluoro-4′-[5-(hydroxymethyl)isoxazol-3-yl]biphenyl-4-yl}-2-oxo-1,3-oxazolidin-5-yl)methyl]acetamide (Compound No. 44),
N-[((5S)-3-{3,5-difluoro-4-[6-(4-pyridin-3-yl-1H-imidazol-1-yl)pyridin-3-yl]phenyl}-2-oxo-1,3-oxazolidin-5-yl)methyl]acetamide (Compound No. 45),
N-[((5S)-3-{3,5-difluoro-4-[6-(5-phenyl-1H-tetrazol-1-yl)pyridin-3-yl]phenyl}-2-oxo-1,3-oxazolidin-5-yl)methyl]acetamide (Compound No. 46),
N-[((5S)-3-{4-[2-(1H-benzimidazol-1-yl)pyrimidin-5-yl]-3,5-difluorophenyl}-2-oxo-1,3-oxazolidin-5-yl)methyl]acetamide (Compound No. 47),
N-[((5S)-3-{4-[2-(1H-benzimidazol-1-yl)pyrimidin-5-yl]-3-fluorophenyl}-2-oxo-1,3-oxazolidin-5-yl)methyl]acetamide (Compound No. 48),
N-[((5S)-3-{3,5-difluoro-4-[2-(1H-1,2,4-triazol-1-yl)pyrimidin-5-yl]phenyl}-2-oxo-1,3-oxazolidin-5-yl)methyl]acetamide (Compound No. 49),
N-[((5S)-3-{3-fluoro-4-[2-(4-phenyl-1H-imidazol-1-yl)pyrimidin-5-yl]phenyl}-2-oxo-1,3-oxazolidin-5-yl)methyl]acetamide (Compound No. 50),
N-[((5S)-3-{3-fluoro-4-[2-(1H-1,2,4-triazol-1-yl)pyrimidin-5-yl]phenyl}-2-oxo-1,3-oxazolidin-5-yl)methyl]acetamide (Compound No. 51),
N-({(5S)-2-oxo-3-[2,3′,6-trifluoro-4′-(5-phenyl-1H-tetrazol-1-yl)biphenyl-4-yl]-1,3oxazolidin-5-yl}methyl)acetamide (Compound No. 52),
N-[((5S)-3-{3,5-difluoro-4-[2-(4-phenyl-1H-imidazol-1-yl)pyrimidin-5-yl]phenyl}-2-oxo-1,3-oxazolidin-5-yl)methyl]acetamide (Compound No. 53).
N-[((5S)-3-{3,5-difluoro-4-[2-(2-oxo-1,3-oxazolidin-3-yl)pyrimidin-5-yl]phenyl}-2-oxo-1,3-oxazolidin-5-yl)methyl]acetamide (Compound No. 54)
N-[((5S)-3-{3-fluoro-4-[2-(1H-1,2,3-triazol-1-yl)pyrimidin-5-yl]phenyl}-2-oxo-1,3-oxazolidin-5-yl)methyl]acetamide (Compound No. 55)
N-[((5S)-3-{4-[2-(3,5-dimethyl-1H-pyrazol-1-yl)pyrimidin-5-yl]-3-fluorophenyl}-1,3-oxazolidin-5-yl)methyl]acetamide (Compound No. 56)
N-[((5S)-3-{3-fluoro-4-[2-(2-oxo-1,3-oxazolidin-3-yl)pyrimidin-5-yl]phenyl}-2-oxo-1,3-oxazolidin-5-yl)methyl]acetamide (Compound No. 57)
N-{[(5S)-3-(4-{6-[4-(difluoromethyl)-1H-imidazol-1-yl]pyridin-3-yl}-3,5-difluorophenyl)-2-oxo-1,3-oxazolidin-5-yl]methyl}acetamide (Compound No. 58)
N-[((5S)-3-{3-fluoro-4-[2-(3-formyl-1H-pyrrol-1-yl)pyrimidin-5-yl]phenyl}-2-oxo-1,3-oxazolidin-5-yl)methyl]acetamide (Compound No. 59)
N-({(5S)-2-oxo-3-[2,3′,6-trifluoro-4′-(1H-1,2,4-triazol-1-yl)biphenyl-4-yl]-1,3-oxazolidin-5-yl}methyl)acetamide (Compound No. 60)
N-[((5S)-3-{3,5-difluoro-4-[2-(3-formyl-1H-pyrrol-1-yl)pyrimidin-5-yl]phenyl}-2-oxo-1,3-oxazolidin-5-yl)methyl]acetamide (Compound No. 61)
N-[((5S)-2-oxo-3-{2,3′,6-trifluoro-4′-[5-(hydroxymethyl)-4,5-dihydroisoxazol-3-yl]biphenyl-4-yl}-1,3-oxazolidin-5-yl)methyl]acetamide (Compound No. 62)
N-[((5S)-3-{3-fluoro-4-[2-(1H-pyrrol-1-yl)pyrimidin-5-yl]phenyl}-2-oxo-1,3-oxazolidin-5-yl)methyl]acetamide (Compound No. 63)
N-({(5S)-3-[2,3′-difluoro-4′-(1H-1,2,4-triazol-1-yl)biphenyl-4-yl}methyl)acetamide (Compound No. 64)
N-({(5S)-3-[3,5-difluoro-4-(6-{4-[(E)-(methoxyimino)methyl]-1H-imidazol-1-yl}pyridin-3-yl)phenyl]-2-oxo-1,3-oxazolidin-5-yl}methyl)acetamide (Compound No. 65)
N-[((5S)-3-{3,5-difluoro-4-[6-(4-formyl-1H-imidazol-1-yl)pyridin-3-yl]phenyl}-2-oxo-1,3-oxazolidin-5-yl)methyl]acetamide (Compound No. 66)
N-[((5S)-3-{4-[6-(4-cyano-1H-imidazol-1-yl)pyridin-3-yl]-3,5-difluorophenyl}-2-oxo-1,3-oxazolidin-5-yl)methyl]acetamide (Compound No. 67)
methyl 1-[5-(4-{(5S)-5-[(acetylamino)methyl]-2-oxo-1,3-oxazolidin-3-yl}-2,6-difluorophenyl)pyridin-2-yl]-1H-imidazole-4-carboxylate (Compound No. 68)
N-({(5S)-3-[3,5-difluoro-4-(6-{4-[(E)-(hydroxyimino)methyl]-1H-imidazol-1-yl}pyridin-3-yl)phenyl]-2-oxo-1,3-oxazolidin-5-yl}methyl)acetamide (Compound No. 69)
N-({(5S)-3-[2,6-difluoro-4′-(1,3-oxazol-5-yl)biphenyl-4-yl]-2-oxo-1,3-oxazolidin-5-yl}methyl)acetamide (Compound No. 70)
N-({(5S)-3-[2,6-difluoro-4′-(5-methyl-1,3,4-oxadiazol-2-yl)biphenyl-4-yl]-2-oxo-1,3-oxazolidin-5-yl}methyl)acetamide (Compound No. 71)
N-({(5S)-3-[2,6-difluoro-4′-(2-methyl-2H-tetrazol-5-yl)biphenyl-4-yl]-2-oxo-1,3-oxazolidin-5-yl}methyl)acetamide (Compound No. 72)
N-({(5S)-3-[2,6-difluoro-4′-(1-methyl-1H-tetrazol-5-yl)biphenyl-4-yl]-2-oxo-1,3-oxazolidin-5-yl}methyl)acetamide (Compound No. 73)
N-({(5S)-2-oxo-3-[2,3′,6-trifluoro-4′-(1-methyl-1H-tetrazol-5-yl)biphenyl-4-yl]-1,3-oxazolidin-5-yl}methyl)acetamide (Compound No. 74)
N-({(5S)-3-[2,3′-difluoro-4′-(1-methyl-1H-tetrazol-5-yl)biphenyl-4-yl]-2-oxo-1,3-oxazolidin-5-yl}methyl)acetamide (Compound No. 75)
N-({(5S)-2-oxo-3-[2,3′,6-trifluoro-4′-(2-methyl-2H-tetrazol-5-yl)biphenyl-4-yl]-1, oxazolidin-5-yl}methyl)acetamide (Compound No. 76)
N-({(5S)-3-[2,3′-difluoro-4′-(5-methyl-1,3,4-oxadiazol-2-yl)biphenyl-4-yl]-2-oxo-1,3-oxazolidin-5-yl}methyl)acetamide (Compound No. 77)
N-({(5S)-2-oxo-3-[2,3′,6-trifluoro-4′-(5-methyl-1,3,4-oxadiazol-2-yl)biphenyl-4-yl]-1,3-oxazolidin-5-yl}methyl)acetamide (Compound No. 78)
N-[((5S)-3-{3,5-difluoro-4-[2-(1H-1,2,3-triazol-1-yl)pyrimidin-5-yl]phenyl}-2-oxo-1,3-oxazolidin-5-yl)methyl]acetamide (Compound No. 79)
N-{[(5S)-3-(3,5-difluoro-4-{6-[5-(4-fluorophenyl)-1H-tetrazol-1-yl]pyridin-3-yl}phenyl)-2-oxo-1,3-oxazolidin-5-yl]methyl}acetamide (Compound No. 80)
Hydrochloride salt of N-({(55)-3-[4′-(1H-benzimidazol-2-yl)-2,3′-difluorobiphenyl-4-yl]-2-oxo-1,3-oxazolidin-5-yl}methyl)acetamide (Compound No. 81)
N-({(5S)-3-[2,3′-difluoro-4′-[2-methyl-2H-tetrazol-5-yl)biphenyl-4-yl]-2-oxo-1,3-oxazolidin-5-yl}methyl)acetamide (Compound No. 82)
N-[((5S)-3-{3,5-difluoro-4-[5-(2-methyl-2H-tetrazol-5-yl)-2-thienyl]phenyl}-2-oxo-1,3-oxazolidin-5-yl)methyl]acetamide (Compound No. 83)
N-{[(5S)-3-(3,5-difluoro-4-{6-[4-(hydroxymethyl)-1H-imidazol-1-yl]pyridin-3-yl}phenyl)-2-oxo-1,3-oxazolidin-5-yl]methyl}acetamide (Compound No. 84)
N-[((5S)-3-{3,5-difluoro-4-[2-(1H-pyrrol-1-yl)pyrimidin-5-yl]phenyl}-2-oxo-1,3-oxazolidin-5-yl)methyl]acetamide (Compound No. 85)
N-[((5S)-3-{3-fluoro-4-[2-(1H-pyrazol-1-yl)pyrimidin-5-yl]phenyl}-2-oxo-1,3-oxazolidin-5-yl)methyl]acetamide (Compound No. 86)

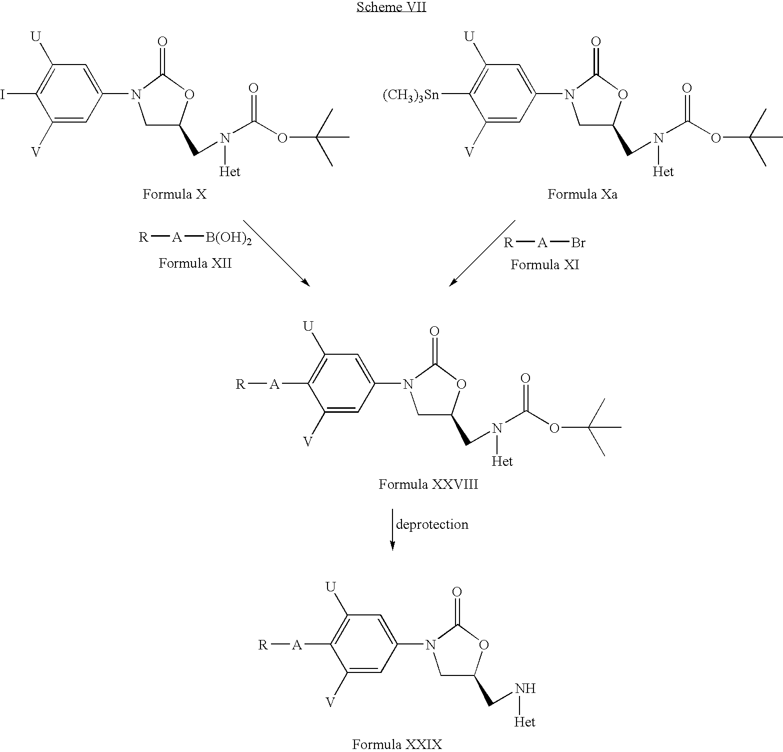

Compounds of Formula XXVIII and Formula XXIX can be prepared following Scheme VII. Thus compounds of Formula X can be reacted with compounds of Formula XII (from Scheme III) to form compounds of Formula XXVIII. Alternatively, compounds of Formula Xa (from Scheme II) can be reacted with compounds of Formula XI to form compounds of Formula XXVIII. Compounds of Formula XXVIII can be deprotected to form compounds of Formula XXIX.

Compounds of Formula X can be cross coupled with compounds of Formula XII; and compounds of Formula Xa can be cross coupled with compounds of Formula XII, to form compounds of Formula XXVIII using one or more bases, for example, carbonates, e.g., sodium carbonate, potassium carbonate, cesium carbonate or mixtures thereof, and in the presence of one or more catalysts, for example, dichlorobistriphenylphosphine palladium (II), tetrakistriphenylphosphine palladium (0), a mixture of palladium diacetate and triphenyl phosphine, or mixtures thereof. These reactions can also be carried out in one or more organic solvents, for example, n-propanol, 1,4-dioxane, acetone or mixtures thereof.

Compounds of Formula XXVIII can be deprotected to form compounds of Formula XXIX in presence of one or more acids, for example, hydrochloric acid in a solvent, for example, ethanol; or trifluoroacetic acid in dichloromethane.

Compounds prepared following Scheme VII include, for example:
tert-butyl [((5R)-3-{3,5-difluoro-4-[6-(1H-1,2,4-triazol-1-yl)pyridin-3-yl]phenyl}-2-oxo-1,3-oxazolidin-5-yl)methyl]isoxazol-3-ylcarbamate (Compound No. 87),
tert-butyl [((5R)-3-{3-fluoro-4-[6-(1,3-oxazol-5-yl)pyridin-3-yl]phenyl}-2-oxo-1,3-oxazolidin-5-yl)methyl]isoxazol-3-ylcarbamate (Compound No. 88),
(5S)-3-{3,5-difluoro-4-[6-(2-methyl-2H-tetrazol-5-yl)pyridin-3-yl]phenyl}-5-[(isoxazol-3-ylamino)methyl]-1,3-oxazolidin-2-one (Compound No. 89),
(5S)-3-[2,3′-difluoro-4′-(1,3-oxazol-5-yl)biphenyl-4-yl]-5-[(isoxazol-3-ylamino)methyl]-1,3-oxazolidin-2-one (Compound No. 90),
(5S)-3-{3,5-difluoro-4-[6-(1H-imidazol-1-yl)pyridin-3-yl]phenyl}-5-[(isoxazol-3-ylamino)methyl]-1,3-oxazolidin-2-one (Compound No. 91),
(5S)-5-[(isoxazol-3-ylamino)methyl]-3-[2,3′,6-trifluoro-4′-[4-phenyl-1H-imidazol-1-yl)biphenyl-4-yl]-1,3-oxazolidin-2-one (Compound No. 92),
(5S)-3-{3,5-difluoro-4-[6-(1-methyl-1H-tetrazol-5-yl)pyridin-3-yl]phenyl}-5-[(isoxazol-3-ylamino)methyl]-1,3-oxazolidin-2-one (Compound No. 93), and

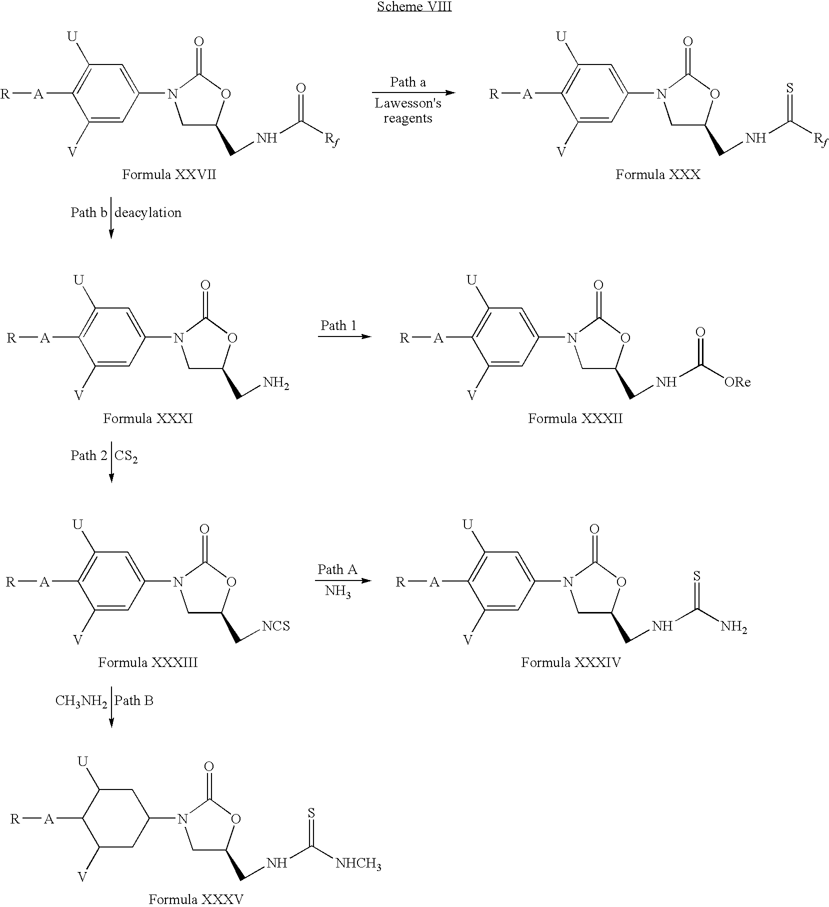

Compounds of Formula XXX, XXXI, XXXII, XXXIII, XXXIV and XXXV can be prepared following Scheme VIII.

Path a: Compounds of Formula XXVII (from Scheme VI) can be reacted with Lawesson's reagent to form compounds of Formula XXX.

Path b: Compounds of Formula XXVII can be deacylated to form compounds of Formula XXXI.

Path 1: Compounds of Formula XXXI can be reacted with alkylchloroformate, for example, methyl chloroformate, to form compounds of Formula XXXII (wherein Rₑ can be alkyl).

Path 2: Compounds of Formula XXXI can be reacted with CS₂ to form compounds of Formula XXXIII. Compounds of Formula XXXIII can be reacted with methanolic ammonia to form compounds of Formula XXXIV (Path A). Compounds of Formula XXXIII can also be reacted with methylamine to form compounds of Formula XXXV (Path B).

Compounds of Formula XXVII can be reacted with Lawesson's reagent to form compounds of Formula XXX (Path a) in one or more organic solvents, for example, dioxane, diethyl ether, dimethylformamide or tetrahydrofuran.

Compounds of Formula XXVII can be deacylated to form compounds of Formula XXXI (Path b) in the presence of one or more acids, for example, hydrochloride acid. This reaction can also be carried out in one or more organic solvents, for example, absolute ethanol, absolute methanol, absolute propanol or mixtures thereof.

Compounds of Formula XXXI can be reacted with methyl chloroformate to form compounds of Formula XXXII (Path 1) in one or more organic solvents, for example, dichloromethane, dichloroethane, carbon tetrachloride, tetrahydrofuran or mixtures thereof.

Compounds of Formula XXXI can be reacted with carbon disulfite to form compounds of Formula XXXIII (Path 2) using one or more bases, for example, nitrogen-containing compounds, e.g., triethylamine, 4-dimethylamino pyridine or N-methyl morpholine. This reaction can also be carried out in one or more organic solvents, for example, tetrahydrofuran, dimethylformamide, carbon tetrachloride or mixtures thereof.

The reaction of compounds of Formula XXXIII to form compounds of Formula XXXIV can be carried out with methanolic ammonia (Path A) in one or more organic solvents, for example methanol, ethanol, propanol or mixtures thereof.

The reaction of compounds of Formula XXXIV with methylamine to form compounds of Formula XXXV (Path B) can be carried out using one or more bases, for example, nitrogen-containing compounds, e.g., triethylamine, diisopropylamine, pyridine or mixtures thereof, and in presence of methyl amine (Path B). This reaction can also be carried out in one or more organic solvents, for example, methanol, ethanol, propanol or mixtures thereof.

Exemplary compounds prepared following Scheme VIII include, for example:
N-[((5S)-3-{3,5-difluoro-4-[6-(1H-1,2,4-triazol-1-yl)pyridin-3-yl]phenyl}-2-oxo-1,3-oxazolidin-5-yl)methyl]ethanethioamide (Compound No. 94),
N-({(5S)-3-[2,3′-difluoro-4′-(1,3-oxazol-5-yl)biphenyl-4-yl]-2-oxo-1,3-oxazolidin-5-yl}methyl)ethanethioamide (Compound No. 95),
(5S)-5-(aminomethyl)-3-{3,5-difluoro-4-[6-(1H-1,2,4-triazol-1-yl)pyridin-3-yl]phenyl}-1,3-oxazolidin-2-one (Compound No. 96),
methyl [((5S)-3-{3,5-difluoro-4-[6-(1H-1,2,4-triazol-1-yl)pyridin-3-yl]phenyl}-2-oxo-1,3-oxazolidin-5-yl)methyl]carbamate (Compound No. 97),
methyl ({(5S)-3-[2,3′-difluoro-4′-(1,3-oxazol-5-yl)biphenyl-4-yl]-2-oxo-1,3-oxazolidin-5-yl}methyl)carbamate (Compound No. 98),
(5S)-3-{3,5-difluoro-4-[6-(1,3-oxazol-5-yl)pyridin-3-yl]phenyl}-5-(isothiocyanatomethyl)-1,3-oxazolidin-2-one (Compound No. 99),
(N-[((5S)-3-{3,5-difluoro-4-[6-(1H-1,2,4-triazol-1-yl)pyridin-3-yl]phenyl}-2-oxo-1,3-oxazolidin-5-yl)methyl]thiourea (Compound No. 100), and
N-[((5S)-3-{3,5-difluoro-4-[6-(1H-1,2,4-triazol-1-yl)pyridin-3-yl]phenyl}-2-oxo-1,3-oxazolidin-5-yl)methyl]-N′-methylthiourea (Compound No. 101).

TABLE I
Formula I

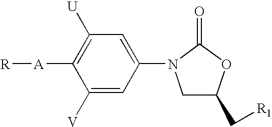

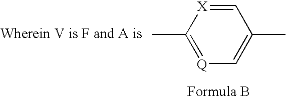

| Compound | | | | | |
|---|---|---|---|---|---|
| No. | Q | R | U | R₁ | X |
| 1 | N | 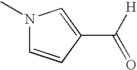 | F | NHCOCH₃ | CH |
| 2 | N | 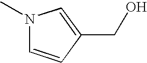 | F | NHCOCH₃ | CH |
| 3 | N | 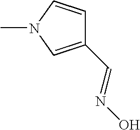 | H | —NHCOCH₃ | N |
| 4 | N | 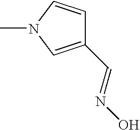 | F | —NHCOCH₃ | N |
| 5 | CF | 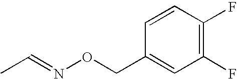 | F | NHCOCH₃ | CH |
| 6 | CF | 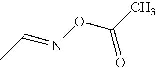 | F | NHCOCH₃ | CH |
| 7 | CF | 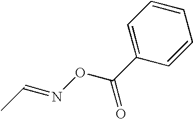 | F | NHCOCH₃ | CH |
| 8 | CF | 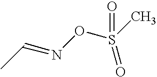 | F | NHCOCH₃ | CH |
| 9 | CF | 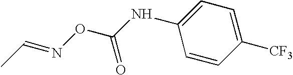 | F | NHCOCH₃ | CH |
| 10 | CF | 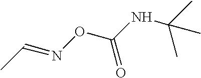 | F | NHCOCH₃ | CH |
| 11 | CF | 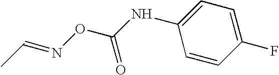 | F | NHCOCH₃ | CH |
| 12 | CF | 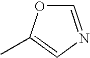 | F | NHCOCH₃ | CH |
| 13 | N | 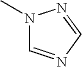 | F | NHCOCH₃ | CH |
| 16 | CF | 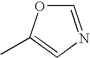 | F | NHCOCH₃ | CH |
| 17 | N | 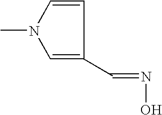 | F | NHCOCH₃ | CH |
| 18 | CF | 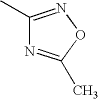 | H | NHCOCH₃ | CH |
| 19 | CF | 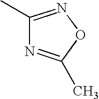 | F | NHCOCH₃ | CH |
| 20 | N | 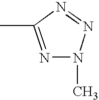 | F | NHCOCH₃ | CH |
| 21 | N | 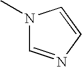 | F | NHCOCH₃ | CH |
| 22 | N | 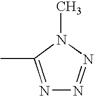 | F | NHCOCH₃ | CH |
| 23 | N | 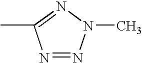 | F | 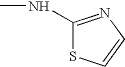 | CH |
| 24 | CF | 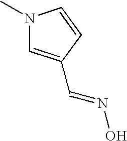 | H | NHCOCH₃ | CH |
| 25 | CF | 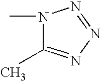 | H | NHCOCH₃ | CH |
| 26 | N | 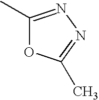 | F | NHCOCH₃ | CH |
| 27 | N | 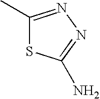 | F | NHCOCH₃ | CH |
| 28* | CF | 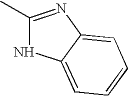 | F | NHCOCH₃ | CH |
| 29 | N | 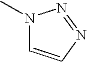 | F | NHCOCH₃ | CH |
| 30 | N | 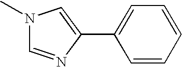 | F | NHCOCH₃ | CH |
| 34 | CF | 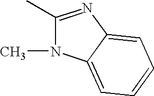 | F | NHCOCH₃ | CH |
| 35 | CF | 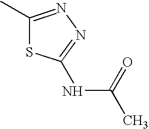 | F | NHCOCH₃ | CH |
| 36 | CF | 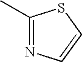 | H | NHCOCH₃ | CH |
| 37 | CF | 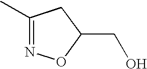 | H | NHCOCH₃ | CH |
| 38 | N | 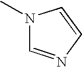 | H | NHCOCH₃ | N |
| 39 | CF | 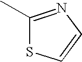 | F | NHCOCH₃ | CH |
| 40 | CF | 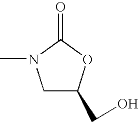 | F | NHCOCH₃ | CH |
| 41 | CF | 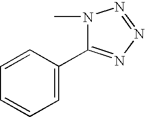 | H | NHCOCH₃ | CH |
| 42 | N | 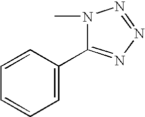 | H | NHCOCH₃ | CH |
| 43 | CF | 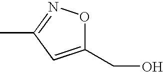 | F | NHCOCH₃ | CH |
| 44 | CF | 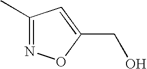 | H | NHCOCH₃ | CH |
| 45 | N | 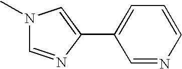 | F | NHCOCH₃ | CH |
| 46 | N | 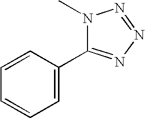 | F | NHCOCH₃ | CH |
| 47 | N | 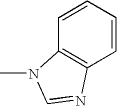 | F | NHCOCH₃ | N |
| 48 | N | 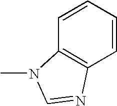 | H | NHCOCH₃ | N |
| 49 | N | 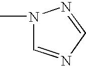 | F | NHCOCH₃ | N |
| 50 | N | 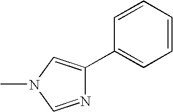 | H | NHCOCH₃ | N |
| 51 | N | 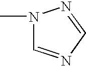 | H | NHCOCH₃ | N |
| 52 | CF | 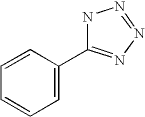 | F | NHCOCH₃ | CH |
| 53 | N | 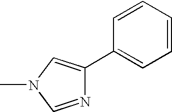 | F | NHCOCH₃ | N |
| 54 | N | 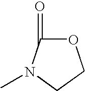 | F | —NHCOCH₃ | N |
| 55 | N | 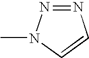 | H | —NHCOCH₃ | N |
| 56 | N | 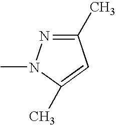 | H | —NHCOCH₃ | N |
| 57 | N | 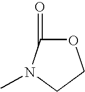 | H | —NHCOCH₃ | N |
| 58 | N | 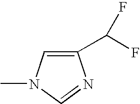 | F | —NHCOCH₃ | CH |
| 59 | N | 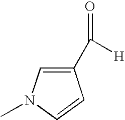 | H | —NHCOCH₃ | N |
| 60 | —CF— | 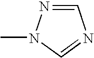 | F | —NHCOCH₃ | CH |
| 61 | N | 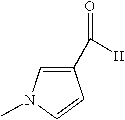 | F | —NHCOCH₃ | N |
| 62 | —CF— | 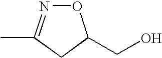 | F | —NHCOCH₃ | CH |
| 63 | N | 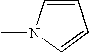 | H | —NHCOCH₃ | N |
| 64 | —CF— | 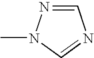 | H | —NHCOCH₃ | CH |
| 65 | N | 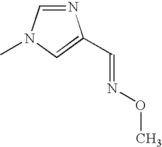 | F | —NHCOCH₃ | CH |
| 66 | N | 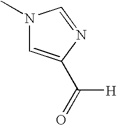 | F | —NHCOCH₃ | CH |
| 67 | N | 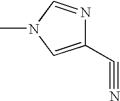 | F | —NHCOCH₃ | CH |
| 68 | N | 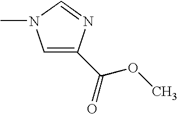 | F | —NHCOCH₃ | CH |
| 69 | N | 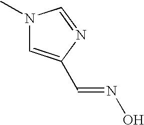 | F | —NHCOCH₃ | CH |
| 70 | —CH— | 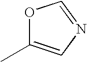 | F | —NHCOCH₃ | CH |
| 71 | —CH— | 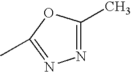 | F | —NHCOCH₃ | CH |
| 72 | —CH— | 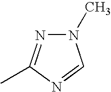 | F | —NHCOCH₃ | CH |
| 73 | —CH— | 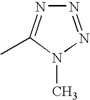 | F | —NHCOCH₃ | CH |
| 74 | —CF— | 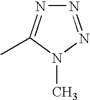 | F | —NHCOCH₃ | CH |
| 75 | —CF— | 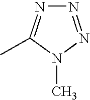 | H | —NHCOCH₃ | CH |
| 76 | —CF— | 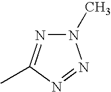 | F | —NHCOCH₃ | CH |
| 77 | —CF— | 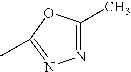 | H | —NHCOCH₃ | CH |
| 78 | —CF— | 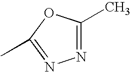 | F | —NHCOCH₃ | CH |
| 79 | N | 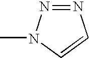 | F | —NHCOCH₃ | N |
| 80 | N | 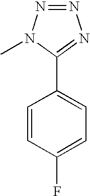 | F | —NHCOCH₃ | CH |
| 81 | —CF— | 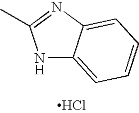 | H | —NHCOCH₃ | CH |
| 82 | —CF— | 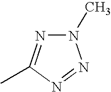 | H | —NHCOCH₃ | CH |
| 84 | N | 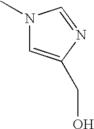 | F | —NHCOCH₃ | CH |
| 85 | N | 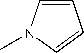 | F | —NHCOCH₃ | N |
| 86 | N | 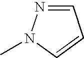 | H | —NHCOCH₃ | N |
| 87 | N | 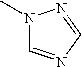 | F | 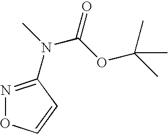 | CH |
| 88 | N | 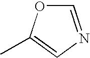 | H | 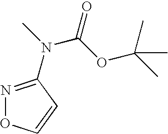 | CH |
| 89 | N | 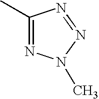 | F | 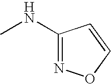 | CH |
| 90 | CF | 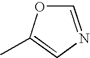 | H | 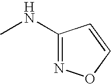 | CH |
| 91 | N | 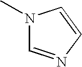 | F | 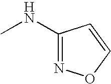 | CH |
| 92 | N | 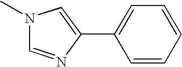 | F | 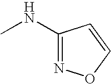 | CH |
| 93 | N | 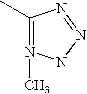 | F | 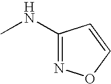 | CH |
| 94 | N | 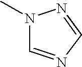 | F | —NHCSCH₃ | CH |
| 95 | CF | 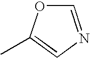 | H | —NHCSCH₃ | CH |
| 96 | N | 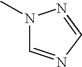 | F | —NH₂ | CH |
| 97 | N | 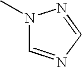 | F | NHCO₂CH₃ | CH |
| 98 | CF | 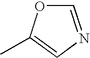 | H | NHCO₂CH₃ | CH |
| 99 | N | 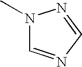 | F | —NCS | CH |
| 100 | N | 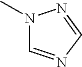 | F | NHCSNH₂ | CH |
| 101 | N | 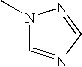 | F | NHCSNHCH₃ | CH |

*represents Hydrochloride salt.

TABLE II
Formula I

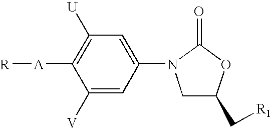

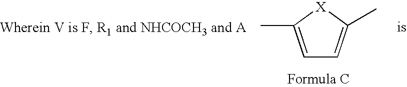

| Compound | | | |
|---|---|---|---|
| No. | X | R | U |
| 14 | O | 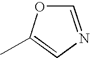 | F |
| 15 | S | 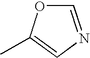 | F |
| 31 | O | 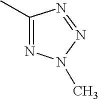 | H |
| 32 | O | 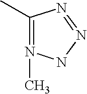 | H |
| 33 | O | 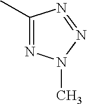 | F |
| 83 | S | 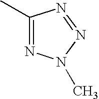 | F |

While the present invention has been described in terms of its specific embodiments, certain modifications and equivalents will be apparent to those skilled in the art and are included within the scope of the present invention. The examples are provided to illustrate particular aspects of the disclosure and do not limit the scope of the present invention as defined by the claims.

### EXAMPLES

### Synthesis of Starting Compounds

### Synthesis of 5-(4-Bromo-2-fluoro-phenyl)-oxazole

4-bromo-2-fluorobenzaldehyde (1.25 g) and tosylmethyl isocyanide (1.3 g) were dried at high vacuum and back filled with argon and then methanol (50 mL) was added to obtain a clear solution. To the reaction mixture was added potassium carbonate (1.85 g) and the reaction mixture was refluxed under argon at 70° C. for about 2.5 hours. Volatiles were removed under vacuum and the crude residue was chromatographed over silica gel column using dichloromethane as eluant to yield the title compound as pure white solid (1.4 g).

EIMS (m/z): 242.36 (M+H)

### Synthesis of 2-(4-Bromo-2-fluoro-phenyl)-1H-benzimidazole

To a solution of 4-bromo-2-fluorobenzaldehyde (4.04 g) in ethanol (20 mL) was added 40% aqueous sodium bisulfite (20 mL) and the reaction mixture was stirred for 1 hour at 30° C. To the reaction mixture was added a solution of phenylenediamine (2.0 g) in ethanol (50 mL) and the reaction mixture was stirred at reflux for about 4 hours. Solvent was removed under vacuum and the product thus obtained was dissolved in water and the reaction mixture was stirred for 10 minutes. A precipitate separated out and was filtered and dried to yield the title compound (4.9 g).

EIMS (m/z): 291.3 (M+H)

### Synthesis of 5-Bromo-2-[1,2,4]triazol-1-yl-pyridine

A solution of 2,5-dibromopyridine (1.0 g), N-methyl-2-pyrrolidin-2-one (10 mL) and 1,2,4-triazole (584 mg) was stirred at room temperature for 15 minutes. The reaction mixture was then heated at 100° C. for about 8 hours, cooled to room temperature, water (50 mL) was added and the precipitate separated out was extracted with ethyl acetate. Solvent was removed under vacuum to afford a viscous material, which was then dissolved in methanol (5 mL) and treated with water (20 mL). The solid separated was filtered and dried under vacuum to yield the title compound (615 mg).

EIMS (m/z) 226.34 (M+H)

### Synthesis of 5-(5-Bromo-furan-2-yl)-oxazole

5-bromofuran-2-carboxaldehyde (1.0 g) and tosylmethyl isocyanide (1.0 g) were dried under vacuum, back filled with argon and then methanol (20 mL) was added to obtain a clear solution. To it was added potassium carbonate (723 mg) and the reaction mixture was refluxed under argon for about 2 hours. Volatiles were removed under vacuum and the crude residue was purified by column chromatography over silica gel column using dichloromethane as eluant to yield the title compound (650 mg).

EIMS (m/z): 214.34 (M+H).

### Synthesis of 5-(5-Bromo-2-thienyl)-1,3-oxazole

5-bromothiophene-2-carboxaldehyde (2.0 g) and tosylmethyl isocyanide (2.2 g) were dried under vacuum and back filled with argon and methanol (200 mL) was added to obtain a clear solution. To it was added potassium carbonate (1.58 g) and the reaction mixture refluxed under argon for about 2 hours. Volatiles were removed under vacuum and the residue was purified by silica gel column using dichloromethane as eluant to yield the title compound (1.4 g)

EIMS (m/z): 230.13 (M+H)

### Synthesis of 5-bromo-2-imidazolyl-pyridine

To a solution of 2,5-dibromopyridine (1.0 g), imidazole (0.574 g) in N-methyl-2-pyrrolidone was added potassium carbonate (1.76 g) at room temperature. The reaction mixture was refluxed at 110-120° C. overnight. The reaction mixture was quenched with water (20 mL) and then extracted with ethyl acetate. Volatiles were removed under vacuum and the residue obtained was triturated with water. The solid separated out was filtered and dried to yield the title compound (0.8 g).

EIMS (m/z) 224 (M+H)

### Synthesis of 5-Bromo-2-[3-carboxaldehydepyrrol-1-yl]-pyridine

A solution of 2-amino-5-bromo-pyridine (1.0 g) and 2,5-dimethoxy-3-carboxaldehyde-tetrahydrofuran (1.29 g) in acetic acid (10 mL) was refluxed at 110-120° C. for about 2 hours. Solvent was removed under vacuum and the residue obtained was azeotroped with toluene (100 mL). The crude product was purified by column chromatography using 2-5% ethyl acetate in hexane as eluant to yield the title compound (550 mg).

EIMS (m/z): 251 (M+H)

### Synthesis of 5-Bromo-2-[3-hydroxyliminomethylpyrrol-1-yl]-pyridine

To a solution of 5-Bromo-2-[3-carboxaldehydepyrrol-1-yl]pyridine (450 mg) in ethanol (20 mL) was added hydroxylamine hydrochloride (248 mg) and the reaction mixture was stirred at room temperature for 5-6 hours. Volatiles were removed under vacuum and the residue obtained was treated with water, solid separated was filtered and dried to yield the title compound (420 mg).

EIMS (m/z): 266.11 (M+H)

### Synthesis of 5-bromo-2-(1-methyl-1H-tetrazol-5-yl)pyridine (A) and 5-bromo-2-(2-methyl-1H-tetrazol-5-yl)pyridine (B)

Step a: Synthesis of 5-bromo-2-(tetrazol-5-yl)pyridine

To a solution of 5-bromopyridine-2-carbonitrile (1.5 g) in toluene was added sodium azide (1.33 g) and triethylamine hydrochloride (2.89 g). The reaction mixture was stirred overnight at 100-110° C., filtered and washed with methanol. The solvent was removed under vacuum to yield the title compound (1.8 g).

### Step b: Synthesis of 5-bromo-2-(1-methyl-1H-tetrazol-5-yl)pyridine and 5-bromo-2-(2-methyl-1H-tetrazol-5-yl)pyridine

To a solution of 5-bromo-2-(tetrazol-5-yl)pyridine (2 g) obtained from step a above in dimethylformamide (20 mL) was added potassium hydroxide (1 g) and methyl iodide (3.18 g) and the reaction mixture was stirred at room temperature for three hours. The reaction mixture was quenched with water, extracted with dichloromethane and the organic layer was dried over anhydrous sodium sulfate. The solvent was removed under vacuum and purified by column chromatography using ethyl acetate (20%) in hexane as eluant to yield 5-bromo-2-(1-methyl-1H-tetrazol-5-yl)pyridine (A) (200 mg) and 5-bromo-2-(2-methyl-1H-tetrazol-5-yl)pyridine (B) (600 mg).

EIMS (m/z): 240.19 (M+H) (A)

EIMS (m/z): 240.18 (M+H) (B)

### Synthesis of 5-bromo-2-(5-methyl-1,3,4-oxadiazol-2-yl)pyridine

### Step a: Synthesis of 5-bromo-2-(tetrazol-5-yl)pyridine

To a solution of 5-bromopyridine-2-carbonitrile (1.5 g) in toluene was added sodium azide (1.33 g) and triethylamine hydrochloride (2.89 g) and the reaction mixture was stirred at 100-110° C. overnight. The reaction mixture was filtered and the solid was washed with methanol. The filtrate was concentrated under vacuum to yield the title compound (1.8 g).

### Step b: Synthesis of 5-bromo-2-(5-methyl-1,3,4-oxadiazol-2-yl)pyridine

To a compound (500 mg) obtained from step a above was added acetic anhydride (10 mL) and refluxed for 5-7 hours. The solvent was evaporated and the residue was taken in dichloromethane, washed with brine and dried over anhydrous sodium sulfate. The solvent was evaporated and the residue was purified by column chromatography using 10% ethyl acetate in hexane as eluant to yield the title compound (200 mg).

EIMS (m/z): 240 (M+H)

### Synthesis of N-[5-(4-bromo-2-fluorophenyl)-1,3,4-thiadiazol-2-yl]acetamide

### Step a: Synthesis of 5-(4-bromo-2-fluorophenyl)-1,3,4-thiadiazol-2-amine

To a solution of 4-bromo-2-fluoro-benzonitrile (2 g) in trifluoroacetic acid (25 mL) was added thiosemicarbazide (0.91 g) and refluxed for 15 hours. The solvent was evaporated, the compound was taken in ethyl acetate and neutralized with sodium bicarbonate solution. The aqueous layer separated out and was dried with anhydrous sodium sulfate and concentrated to yield the title compound (1.35 g).

### Step b: Synthesis of N-[5-(4-bromo-2-fluorophenyl)-1,3,4-thiadiazol-2-yl]acetamide

To a solution of the compound (550 mg) obtained from step a above in dichloromethane (100 mL) was added acetic anhydride (0.4 mL) and triethylamine (0.56 mL). The reaction mixture was stirred at room temperature for 24 hours. The solvent was evaporated and the residue was taken into dichloromethane washed with brine and dried over anhydrous sodium sulfate. The solvent was concentrated and purified by column chromatography using 1% methanol in dichloromethane as eluant to yield the title compound (380 mg).

EIMS (m/z): 316 (M+H)

### Synthesis of 2-(4-bromo-2-fluorophenyl)-1,3-thiazole

### Step a: Synthesis of 4-bromo-2-fluorobenzenecarbothioamide

Hydrogen sulfide gas was passed to a solution of 4-bromo-2-fluorobenzonitrile (5 g) in pyridine (50 mL) and triethylamine (3 mL) for 15 hours at room temperature. The reaction mixture was diluted with dichloromethane, washed with a solution of sodium hydrogen carbonate, brine and dried over anhydrous sodium sulfate. The solvent was concentrated to yield yellow colored title compound (4.5 g).

### Step b: Synthesis of 2-(4-bromo-2-fluorophenyl)-1,3-thiazole

To a solution of compound (4 g) obtained from the step a above in ethanol (50 mL) was added 20% aqueous chloroacetaldehyde (8 mL) and refluxed at 80° C. for 15 hours. The solvent was evaporated and the residue was taken into dichloromethane and washed with brine and dried over anhydrous sodium sulfate. The solvent was concentrated and purified by column chromatography using 30% dichloromethane in hexane as eluant to yield the title compound (1.5 g)

EIMS (m/z): 258 (M+H)

### Synthesis of 1-(4-bromo-2-fluorophenyl)-5-methyl-1H-tetrazole

To a solution of 4-bromo-2-fluoroaniline (2 g) in acetic acid was added triethylorthoacetate (3.09 mL) and sodium azide (1.02 g). The reaction mixture was refluxed for 2 hours. Volatiles were removed under vacuum and the residue was taken into dichloromethane and washed with brine solution, dried over anhydrous sodium sulfate and concentrated. Trituration over hexane afforded the product as white solid (780 mg).

EIMS (m/z): 257 (M+H)

### Synthesis of [3-(4-bromo-2-fluorophenyl)-4,5-dihydroisoxazol-5-yl]methanol

### Step a: Synthesis of 4-bromo-2-fluorobenzaldehyde oxime

To a solution of 4-bromo-2-fluorobenzaldehyde (4.04 g) in ethanol (50 mL) was added hydroxylamine hydrochloride (2.08 g). The reaction mixture was stirred at room temperature for 1 hour and filtered to yield the title compound (4 g).

### Step b: Synthesis of [3-(4-bromo-2-fluorophenyl)-4,5-dihydroisoxazol-5-yl]methanol

To the compound (217 mg) obtained from the step a above in tetrahydrofuran (2 mL) was added allyl alcohol (145 mg) and sodium hypochlorite solution (6.75 mL). The reaction mixture was stirred overnight at room temperature, extracted with ethyl acetate and washed with sodium bicarbonate solution. The solvent was removed to afford a thick slurry, which was then purified by column chromatography using 30% ethyl acetate-hexane as eluant to afford the title compound (150 mg).

EIMS (m/z): 274 (M+H).

### Synthesis of 5-bromo-2-(1H-imidazol-1-yl)pyrimidine

To a solution of 5-bromo-2-chloropyrimidine (1.02 g) in N-methylpyrrolidine-2-one (10 mL) was added 1H-imidazole (680 mg) and potassium carbonate (2.74 g) and refluxed overnight at 80° C. The reaction mixture was poured in water and extracted with dichloromethane, washed with brine and dried over anhydrous sodium sulfate. Solvent was removed under vacuum and to the product thus obtained was added cold ethanol which afforded the title compound (200 mg).

EIMS (m/z): 225 (M+H)

### Synthesis of 1-(5-bromopyrimidin-2-yl)-1H-benzimidazole

To a solution of 5-bromo-2-chloropyrimidine (0.95 g) in N-methylpyrrolidin-2-one (10 mL) was added 1H-benzimidazole (578 mg), potassium carbonate (1.36 g) and the reaction mixture was stirred overnight at 80° C. The reaction mixture was poured into ice-cooled water, precipitate which separated out was filtered and dried to yield the title compound (350 mg).

EIMS (m/z): 275 (M+H)

### Synthesis of 5-bromo-2-(1H-1,2,4-triazol-1-yl)pyrimidine

To a solution of 5-bromo-2-chloropyrimidine (1.0 g) in N-methylpyrrolidin-2-one (10 mL) was added 1H-1,2,4-triazole (359 mg), potassium carbonate (1.45 g) and the reaction mixture was stirred overnight at 60° C. The reaction mixture was poured in water and extracted with dichloromethane, washed with brine. The organic layer was dried over anhydrous sodium sulfate, concentrated and triturated over cold ethanol to yield the title compound (500 mg)

EIMS (m/z): 225 (M+H)

### Synthesis of 5-bromo-2-(4-phenyl-1H-imidazol-1-yl)pyrimidine

To a solution of 5-bromo-2-chloropyrimidine (1.15 g) in N-methylpyrrolidin-2-one (10 mL) was added 4-phenyl-1H-imidazole (865 mg) and potassium carbonate (1.6 g). The reaction mixture was stirred overnight at 80° C. The reaction mixture was poured in water, extracted with dichloromethane and washed with brine. The organic layer was dried over anhydrous sodium sulfate and concentrated. The crude was triturated over cold ethanol to afford the title compound (550 mg).

EIMS (m/z): 301 (M+H)

### Synthesis of 1-(4-bromo-2-fluorophenyl)-5-phenyl-1H-tetrazole

### Step a: Synthesis of N-(4-bromo-2-fluorophenyl)benzamide

To a solution of 4-bromo-2-fluoroaniline (5 g) in dichloromethane (100 mL) at 0° C. was added triethylamine (8.11 mL) and the reaction mixture was stirred for 1 hour followed by the addition of benzoyl chloride (7.36 g) and a catalytic amount of 4-dimethylaminopyridine (0.05 g). The resulting reaction mixture was stirred overnight at room temperature. The reaction mixture was poured in a solution of sodium hydrogen carbonate and filtered. The organic layer was dried over anhydrous sodium sulfate and concentrated to afford crude product, which was recrystallized with hexane to yield the title compound (13 g).

### Step b: Synthesis of 1-(4-bromo-2-fluorophenyl)-5-phenyl-1H-tetrazole

To a compound (2.9 g) obtained from the step a above in toluene (100 mL) was added phosphorous pentachloride (3.16 g) and reaction mixture was refluxed for 15 hours. The solvent was evaporated under reduced pressure and the reaction mixture was poured into a precooled solution of acetone (30 mL). A precooled solution of water (25 mL) with sodium azide (1.3 g) and sodium acetate (1.64 g) was added into the solution of acetone. The reaction mixture was stirred for 12 hours at room temperature. The solvent was removed and residue extracted with dichloromethane, dried over anhydrous sodium sulfate and the solvent was evaporated. The crude product obtained was recrystallized from hexane: diethyl ether mixture (50:50) to yield the title compound (900 mg).

EIMS (m/z): 319 (M+H)

### Synthesis of 5-bromo-2-(5-phenyl-1H-tetrazol-1-yl)pyridine

### Step a: Synthesis of N-(5-bromopyridin-2-yl)benzamide

*To a solution of 5-bromo-2-aminopyridine (5 g) in dichloromethane (100 mL) at 0° C. was added triethylamine (8.11 mL) and the reaction mixture was stirred for 1 hour followed by the addition of benzoyl chloride (7.36 g) and a catalytic amount of 4-dimethylaminopyridine (0.05 g). The reaction mixture stirred overnight at room temperature was poured into a solution of sodium hydrogen carbonate and filtered. The organic layer was dried over anhydrous sodium sulfate and concentrated to afford crude product, which was recrystallized with hexane to yield the title compound (11 g).

### Step b: Synthesis of 5-bromo-2-(5-phenyl-1H-tetrazol-1-yl)pyridine

To a compound (2.9 g) obtained from the step a above in toluene (100 mL) was added phosphorous pentachloride (3.16 g) and refluxed for 15 hours. The solvent was evaporated under reduced pressure and the reaction mixture was poured into a precooled solution of acetone (30 mL). A precooled solution of water (25 mL) with sodium azide (1.3 g) and sodium acetate (1.64 g) was added into the solution of acetone and the reaction mixture and was stirred for 12 hours at room temperature. The solvent was removed and resulting residue extracted with dichloromethane and dried over anhydrous sodium sulfate. The solvent was evaporated and the crude product obtained was recrystallized from hexane:diethyl ether mixture to yield the title compound (850 mg).

EIMS (m/z): 302 (M+H)

### Synthesis of [3-(4-bromo-2-fluorophenyl)isoxazol-5-yl]methanol

### Step a: Synthesis of 4-bromo-2-fluorobenzaldehyde oxime

To a solution of 4-bromo-2-fluorobenzaldehyde (4.04 g) in ethanol (50 mL) was added hydroxylamine hydrochloride (2.08 g). The reaction mixture was stirred at room temperature for 1 hour and filtered to yield the title compound (4 g).

### Step b: Synthesis of [3-(4-bromo-2-fluorophenyl)isoxazol-5-yl]methanol

To the compound (300 mg) obtained from the step a above in tetrahydrofuran (2 mL) was added propargyl alcohol (0.2 mL) and sodium hypochloride (6.75 mL) and the reaction mixture was stirred at room temperature overnight. The reaction mixture was extracted with ethyl acetate and washed with sodium bicarbonate solution. The solvent was removed to afford a viscous slurry, which was purified by column chromatography using 30% ethyl acetate in hexane as eluant to afford the title compound (100 mg).

EIMS (m/z): 272 (M+H)

### Synthesis of 5-bromo-2-(4-pyridine-3-yl-1H-imidazol-1-yl)pyridine

To a solution of 2,5-dibromopyridine (1 g) in N-methylpyrrolidine-2-one (10 mL) was added 3-(1H-imidazol-4-yl)-pyridine and the reaction mixture was stirred for 8 hours at reflux temperature. The reaction mixture was cooled to room temperature, poured into water and extracted with ethyl acetate. The organic layer was dried over sodium sulfate, and concentrated to form a thick emulsion. The emulsion was dissolved in methanol and treated with water to separate out solid product, which was filtered and dried to yield the title compound (615 mg).

EIMS (m/z): 301.11 (M+H)

### Synthesis of (5R)-3-(4-bromo-2-fluorophenyl)-5-(hydroxymethyl)-1,3-oxazolidin-2-one

### Step a: Synthesis of phenyl (4-bromo-2-fluorophenyl)carbamate

A solution of 4-bromo-2-fluoroaniline (5.6 g) in tetrahydrofuran (100 mL) was cooled to 5° C. and to it was added sodium bicarbonate (8.4 g). The reaction mixture was stirred at room temperature for 4 hours along with dropwise addition of benzylchloroformate (5.95 g). The reaction mixture was filtered and concentrated. The residue thus obtained was dissolved in ethyl acetate and washed with saturated sodium bicarbonate and brine solution. The organic layer was dried over anhydrous sodium sulfate and concentrated to yield the title compound (8.5 g)

### Step b: Synthesis of (5R)-3-(4-bromo-2-fluorophenyl)-5-(hydroxymethyl)-1,3-oxazolidin-2-one

To a solution of compound (3.23 g) obtained from the step a above in dry tetrahydrofuran (75 mL) cooled to −78° C. was added n-butyl lithium (6.4 mL). The reaction mixture was stirred at −78° C. for 2 hours. R-(−) glycidyl butyrate (1.73 mL) was slowly added and further stirred at the same temperature for one hour and then stirred overnight at room temperature. The reaction mixture was filtered and to it was added ammonium chloride solution. The organic layer was separated and washed with water, brine and dried over sodium sulfate. The solvent was concentrated and the crude product was purified by column chromatography using 1% methanol-dichloromethane as eluant to yield the title compound (1.5 g)

EIMS (m/z): 290 (M+H)

### Synthesis of 3-(4-Bromo-2-fluoro-phenyl)-5-methyl-[1,2,4]oxadiazole

### Step a: Synthesis of 4-bromo-2-fluorobenzaldehyde oxime

To a solution of 4-bromo-2-fluorobenzaldehyde (4.04 g) in absolute ethanol (100 mL) was added hydroxylamine hydrochloride (2.07 g) and the reaction mixture was stirred at 25° C. for about 5 hours. Volatiles were removed under vacuum and the product thus obtained was poured into water, stirred for 1 hour, and the resulting white crystalline precipitate was filtered and dried to yield the title compound (4.0 g).

EIMS (m/z) 218.28 (M+H)

### Step b: Synthesis of 4-bronco-2-fluorobenzonitrile

The solution of compound (2 g) obtained form step a above in dry acetic anhydride (15 mL) was stirred at 100° C. under argon for 3 hours. The content was dissolved in dichloromethane and washed with dilute sodium bicarbonate solution and dried over sodium sulfate. The solvent was removed under vacuum and the crude product obtained was purified by purified by column chromatography using 20% dichloromethane in hexane as eluant to afford the title compound (540 mg).

EIMS (m/z): 200.08 (M+H)

### Step c: Synthesis of 4-Bromo-2-fluoro-N-hydroxy-benzamidine

To a solution of compound (1.99 g) obtained from step b above, in dry ethanol (20 mL) was added hydroxylamine hydrochloride (1.38 g) and potassium carbonate (2.07 g). The reaction mixture was stirred for 18 hours at reflux temperature. The reaction mixture was cooled and filtered to remove solid impurities. The solvent was removed under vacuum and the crude compound was triturated over diethyl ether and hexane to yield the title product (2 g).

EIMS (m/z): 233 (M+H)

### Step d: Synthesis of 3-(4-Bromo-2-fluoro-phenyl)-5-methyl-[1,2,4]oxadiazole

Molecular sieves (4 Å powder, 3 g) was added to the solution of the compound (1.6 g) obtained from step c above in dry tetrahydrofuran (50 mL) and the reaction mixture was stirred for about 45 minutes. Sodium hydride (suspended in mineral oil, 60% w/w, 0.66 g) was added to the reaction mixture and heated to 60° C. To the reaction mixture, methyl acetate (2.8 g) in dry tetrahydrofuran was added dropwise and refluxed for about 15 hours. Volatiles were removed under vacuum and the product thus obtained was dissolved in dichloromethane (100 mL), washed with water, and the solvent was removed under vacuum to afford a crude product. The crude product was purified by purified by column chromatography using 50% dichloromethane-hexane as eluant to yield the title compound (1 g).

EIMS (m/z): 257.26 (M+H)

### Synthesis of 3-(5-bromopyrimidin-2-yl)-1,3-oxazolidin-2-one

A mixture of 2-oxazolidinone (200 mg), 2-chloro-5-bromopyrimidine (444 mg) and potassium carbonate (638 mg) was taken together and dried under high vacuum for 10 minutes. To this was added N-methylpyrrolidin-2-one (5 mL) and the reaction mixture was stirred at 80° C. for 3 hours. The reaction mixture was poured into crushed ice (200 g), a solid that separated out was filtered and dried under high vacuum overnight to yield the title compound. (350 mg)

EIMS (m/z): 245.05 (M+H)

### Synthesis of 5-bromo-2-(1H-1,2,3-triazol-1-yl)pyrimidine

To a solution of 5-bromo-2-chloropyrimidine (560 mg) in N-methylpyrrolidin-2-one (5 mL) was added 1H-1,2,3-triazole (200 mg) and potassium carbonate (803 mg). The reaction mixture was stirred at 80° C. for 2 hours then poured into cooled water (100 mL). A solid that separated out was filtered and dried under high vacuum to yield the title compound. (400 mg)

EIMS (m/z): 227.04 (M+H)

### Synthesis of 5-bromo-2-(3,5-dimethyl-1H-pyrazol-1-yl)pyrimidine

To a solution of 5-bromo-2-chloropyrimidine (600 mg) in N-methylpyrrolidin-2-one (5 mL) was added 3,5-dimethylpyrazole (298 mg) and potassium carbonate (861 mg). The reaction mixture was stirred at 80° C. for 4 hours and then poured into ice-cooled water (100 mL). The solid that separated out was extracted with dichloromethane. The combined organic layers were dried over anhydrous sodium sulfate. The solvent was removed and the crude product thus obtained was purified by column chromatography using dichloromethane as eluant to yield the title compound (200 mg).

EIMS (m/z): 254.10 (M+H)

### Synthesis of 5-Bromo-2-[3-carboxaldehydepyrrol-1-yl]-pyrimidine

A solution of 2-amino-5-bromo-pyrimidine (2.7 g) and 2,5-dimethoxy-3-carboxaldehyde-tetrahydrofuran (3 g) in acetic acid (100 mL) was refluxed at 110-120° C. for about 2-3 hours. The solvent was removed and the residue obtained was azeotroped with toluene (100 mL). The crude product thus obtained was purified by column chromatography using 2-5% ethyl acetate in hexane as eluant to yield the title compound (1.08 g).

EIMS (m/z): 252.0 (M+H)

### Synthesis of 5-bromo-2-[4-(difluoromethyl)-1H-imidazol-1-yl]pyridine

A compound 1-(5-bromopyridin-2-yl)-1H-imidazole-4-carbaldehyde (600 mg) was dissolved in dichloromethane (15 mL) and the reaction mixture was stirred with diethylaminosulfurtrifluoride (960 mg) at room temperature for 17 hours. The reaction mixture was diluted with dichloromethane (200 mL) and the organic layer was washed with dilute sodium bicarbonate solution (25 mL) and then brine. The organic layer was dried over anhydrous sodium sulfate and the solvent evaporated to yield the title compound (253 mg).

EIMS (m/z): 275.07 (M+H)

### Synthesis of 1-(4-bromo-2-fluorophenyl)-1H-1,2,4-triazole

### Step a: Synthesis of 1-(4-nitro-2-fluorophenyl)-1H-1,2,4-triazole

To a solution of 3,4-difluoronitrobenzene (15.9 g) and potassium hydrogen phosphate (27.2 g) in dimethyl sulfoxide (25 mL) was added 1H-1,2,4-triazole (7.5 g) and the reaction mixture was stirred at 90° C. for 18 hours. The reaction mixture was poured into water (250 mL), extracted with ethyl acetate, washed with water, and resulting the organic layer was dried over anhydrous sodium sulfate. The solvent was evaporated and the crude product was precipitated over hexane to yield the title compound (6 g).

### Step b: Synthesis of 1-(4-amino-2-fluorophenyl)-1H-1,2,4-triazole

To the solution of the compound obtained from the step a above (6 g) in methanol (100 mL) was added Raney nickel (1 g) and the reaction mixture was stirred at room temperature. To the reaction mixture was slowly added hydrazine hydrate (5 mL) over a period of 1 hour. The reaction mixture was filtered over a celite bed and the filtrate was evaporated and the crude product was precipitated over hexane to give title compound (3 g).

### Step c: Synthesis of 1-(4-bromo-2-fluorophenyl)-1H-1,2,4-triazole

The compound obtained from the step b above (1.8 g) and copper bromide (2.85 g) were suspended in 48% aqueous hydrogen bromide solution (50 mL), cooled to −5° C. followed by the slow addition of solid sodium nitrite (2.07 g). The reaction mixture was stirred at −5° C. for 2 hours and neutralized with 20% aqueous sodium hydroxide solution to pH of about 7. A solid that separated out was extracted with dichloromethane and the organic layer was washed with water dried over anhydrous sodium sulfate and the solvent was removed to yield the title compound. (990 mg).

EIMS (m/z): 243.05 (M+H)

### Synthesis of 5-bromo-2-(1H-pyrrol-1-yl)pyrimidine

A solution of 2-amino-5-bromo-pyrimidine (2.0 g) and 2,5-dimethoxy-tetrahydrofuran (2.27 g) in acetic acid (50 mL) was refluxed at 110-120° C. for about 3 hours. The solvent was removed and the residue obtained was azeotroped with toluene (100 mL). The crude product was purified by column chromatography using 2-5% ethyl acetate in hexane as eluant to yield the title compound (1.7 g).

EIMS (m/z): 224.0 (M+H),

### Synthesis of 1-(5-bromopyridin-2-yl)-1H-imidazole-4-carbaldehyde O-methyloxime

The compound 1-(5-bromopyridin-2-yl)-1H-imidazole-4-carbaldehyde (600 mg) was dissolved in a mixture of ethanol (20 mL) and methanol (10 mL) by heating at 40-50° C. for 0.5 hours. To the clear solution was added methyl hydroxylamine hydrochloride (260 mg) and the reaction mixture was stirred at room temperature for 2 hours. Volatiles were removed in vacuo and the residue was taken in water. The solid thus separated was filtered and dried to yield the title compound (500 mg).

EIMS (m/z): 282.11 (M+H).

### Synthesis of 1-(5-bromopyridin-2-yl)-1H-imidazole-4-carbaldehyde

### Step a: Synthesis of 2,3,5-triiodoimidazole

A solution of imidazole (10 g) in aqueous sodium hydroxide (2 M) (360 mL) was added to a solution of iodine (74.6 g) in chloroform (360 mL). The reaction mixture was stirred at room temperature for 12 hours. The organic layer was separated from the aqueous layer and the aqueous layer was neutralized with 50% aqueous acetic acid solution. A solid that separated out was filtered and dried to yield the title compound (48 g).

### Step b: Synthesis of 4-iodo-1H-imidazole

To the compound (35 g) obtained from the step a above in ethanol was added a saturated aqueous solution of sodium sulfite. The reaction mixture was refluxed at 80° C. for 24 hours, filtered and the filtrate was evaporated in vacuo to remove ethanol. The residual aqueous layer was extracted with ethyl acetate, dried over anhydrous sodium sulfate. The solvent removal in vacuo yielded the title compound (5.5 g)

### Step c: Synthesis of 5-bromo-2-(4-iodo-1H-imidazol-1-yl)pyridine

2,5-Dibromopyridine (2 g) and 4-iodo-1H-imidazole (2.45 g) obtained from step b above was dissolved in N-methylpyrrolidin-2-one (5 mL). To the reaction mixture was added anhydrous potassium carbonate (3.5 g) and the reaction mixture was heated overnight at about 100-110° C. The reaction mixture was poured into cooled water and the solid that separated out was filtered and dried over phosphorous pentaoxide to yield the title compound. (670 mg).

### Step d: Synthesis of 1-(5-bromopyridin-2-yl)-1H-imidazole-4-carbaldehyde

To a solution of compound (650 mg) obtained from step c above in dry dichloromethane (10 mL) under argon atmosphere was added ethyl magnesium bromide (1 M solution) (5.4 mL) and the reaction mixture was stirred at room temperature for 0.5 hours. Dry dimethyl formamide (0.38 mL) was added and the reaction mixture was stirred at room temperature for 0.5 hours. The reaction was quenched with aqueous ammonium chloride solution and extracted with dichloromethane. The organic layer was dried over anhydrous sodium sulfate and evaporated in vacuo to yield a crude compound, which was further purified by column chromatography using 5% methanol in dichloromethane as eluant to afford the title compound (680 mg)

EIMS (m/z): 253.07 (M+H)

### Synthesis of 1-(5-bromopyridin-2-yl-1H-imidazole-4-carbonitrile

### Step a: Synthesis of 1-(5-bromopyridin-2-yl)-1H-imidazole-4-carbaldehyde oxime

1-(5-bromopyridin-2-yl)-1H-imidazole-4-carbaldehyde (1.2 g) was dissolved in a mixture of ethanol (20 mL) and methanol (30 mL) by heating at 40-50° C. for 0.5 hours. To the clear solution was added hydroxylamine hydrochloride (430 mg) and the reaction mixture was stirred at room temperature for 3 hours. Volatiles were removed in vacuo and the resulting residue was taken in water. A solid that separated out was filtered and dried to yield the title compound. (1.01 g)

### Step b: Synthesis of 1-(5-bromopyridin-2-yl)-1H-imidazole-4-carbonitrile

1-(5-bromopyridin-2-yl)-1H-imidazole-4-carbaldehyde oxime (1.04 g) was taken in acetic anhydride (10 mL) and the reaction mixture was refluxed at 100-110° C. for 3 hours. Volatiles were removed in vacuo and the residue was diluted with dichloromethane (200 mL) and washed with water. The organic layer was dried over anhydrous sodium sulfate and the solvent was removed to afford the crude product, which was purified by column chromatography using 30% ethyl acetate in hexane as eluant to afford the title compound (500 mg).

EIMS (m/z): 250.07 (M+H)

### Synthesis of methyl 1-(5-bromopyridin-2-yl)-1H-imidazole-4-carboxylate

### Step a: Synthesis of 1-(5-bromopyridin-2-yl-1H-imidazole-4-carboxylic acid

1-(5-bromopyridin-2-yl)-1H-imidazole-4-carbaldehyde (1 g) was dissolved in aqueous sodium carbonate solution (85 mg in 10 mL water). The reaction mixture was stirred and cooled to 5° C. followed by the slow addition of potassium permanganate (820 mg, dissolved in 100 mL water). The reaction mixture was father stirred for 5 hours and filtered through a celite bed. Filtrate was acidified with concentrated sulfuric acid up to pH ˜2 and extracted with ethyl acetate, dried over anhydrous sodium sulfate the solvent was removed to afford the title product. (175 mg)

### Step b: Synthesis of methyl 1-(5-bromopyridin-2-yl-1H-imidazole-4-carboxylate

To the compound (175 mg) obtained from step a above in dimethylformamide (10 mL) was added potassium carbonate (271 mg) and the reaction mixture was cooled to 5° C. followed by the addition of methyl iodide (0.08 mL). The reaction mixture was stirred at room temperature for 4 hours, diluted with dichloromethane (100 mL) and washed with water. The organic layer was dried over anhydrous sodium sulfate and evaporated in vacuo to yield the title compound. (85 mg)

EIMS (m/z): 283.1 (M+H)

### Synthesis of 5-(4-bromophenyl)-1,3-oxazole

A mixture of 4-bromobenzaldehyde (5 g) and tosylmethyl isocyanide (5.3 g) was dried at high vacuum and to it was added methanol (175 mL) to obtain a clear solution. To the reaction mixture was added potassium carbonate (3.7 g) and refluxed under argon at 70° C. for about 2.5 hours. Volatiles were removed under vacuum and the resulting crude residue was purified by column chromatography over silica gel using dichloromethane as eluant to yield the title compound (4.0 g).

EIMS (m/z): 225 (M+H)

### Synthesis of 2-(4-bromophenyl)-5-methyl-1,3,4-oxadiazole

### Step a: Synthesis of 5-bromo-2-(tetrazol-5-yl)phenyl

To a solution of 4-bromobenzonitrile (5.0 g) in dimethylformamide (50 mL) was added sodium azide (4.46 g) and ammonium chloride (4.5 g) and was stirred at 100-110° C. for 4 hours. The reaction mixture was filtered and the filtrate was concentrated to yield the title compound (5.5 g).

### Step b: Synthesis of 2-(4-bromophenyl)-5-methyl-1,3,4-oxadiazole

To a compound (1.0 g) obtained from step a above was added acetic anhydride (20 mL) and refluxed for 4 hours. The solvent was evaporated, the residue was taken in ethyl acetate, washed with brine and dried over anhydrous sodium sulfate. The solvent was evaporated and the residue on triturating over hexane afforded the title compound (700 mg).

EIMS (m/z): 240.07 (M+H)

### Synthesis of 5-(4-bromophenyl)-2-methyl-2H-tetrazole (A) and 5-(4-bromophenyl)-1-methyl-1H-tetrazole (B)

To 5-bromo-2-(tetrazol-5-yl)phenyl (4.0 g) obtained from step a of the above example was added dimethylformamide (70 mL), potassium hydroxide (2.5 g) and methyl iodide (3.8 g). The reaction mixture was stirred at room temperature for 3-4 hours, the reaction was quenched with water, extracted with dichloromethane the organic layer was dried over anhydrous sodium sulfate and the solvent was removed in vacuo to afford a crude mixture of the title compounds, which were separated by column chromatography using 15% ethyl acetate/hexane as eluant to afford 5-(4-bromophenyl)-2-methyl-2H-tetrazole (3.0 g) (A) and 5-(4-bromophenyl)-1-methyl-1H-tetrazole (700 mg) (B)

EIMS (m/z): 240.08 (M+H) (A),

EIMS (m/z): 240.16 (M+H) (B)

### Synthesis of 5-(4-bromo-2-fluorophenyl)-2-methyl-1H-tetrazole (A) and 5-(4-bromo-2-fluorophenyl)-1-methyl-1H-tetrazole (B)

### Step a: Synthesis of 5-(4-bromo-2-fluorophenyl)-1H-tetrazole

To a solution of 4-bromo-2-fluorobenzonitrile (10.0 g) in Toluene (250 mL) was added sodium azide (6.5 g) and triethyl amine hydrochloride (13.7 mL) and the reaction mixture was stirred at 100-110° C. for 5 hours. The reaction mixture was filtered and the solid was washed with methanol. The filtrate was concentrated under vacuum to yield the title compound (14 g).

### Step b: 5-(4-bromo-2-fluorophenyl)-2-methyl-1H-tetrazole and 5-(4-bromo-2-fluorophenyl)-1-methyl-1H-tetrazole

To a compound (14.0 g) obtained from step a above was dissolved in dimethylformamide (30 mL), and KOH (6.4 g) and methyl iodide (10.8 mL) were added. The reaction mixture was stirred at room temperature for 4 hours. Volatiles were removed in vacuo and the product thus obtained was dissolved in dichloromethane, washed with water, and the resulting organic layer was dried over anhydrous sodium sulfate. The solvent was evaporated, the residue was purified by column chromatography using DCM as eluant to yield two products: 5-(4-bromo-2-fluorophenyl)-2-methyl-1H-tetrazole (4 g) (A) and 5-(4-bromo-2-fluorophenyl)-1-methyl-1H-tetrazole (1.5 g) (B).

EIMS (m/z): 258.07 (M+H) (A)

EIMS (m/z): 258.01 (M+H) (B).

### Synthesis of 2-(4-bromo-2-fluorophenyl)-5-methyl-1,3,4-oxadiazole

### Step a: Synthesis of 5-(4-bromo-2-fluorophenyl)-1H-tetrazole

To a solution of 4-bromo-2-fluorobenzonitrile (1.5 g) in toluene was added sodium azide (1.33 g) and triethylamine hydrochloride (2.89 g) and the reaction mixture was stirred at 100-110° C. overnight. The reaction mixture was filtered and the solid was washed with methanol. The filtrate was concentrated under vacuum to yield the title compound (1.8 g).

### Step b: Synthesis of 2-(4-bromo-2-fluorophenyl)-5-methyl-1,3,4-oxadiazole

To a compound (500 mg) obtained from step a above was added acetic anhydride (10 mL) and refluxed for 5-7 hours. The solvent was evaporated, the residue was taken in dichloromethane, washed with brine and dried over anhydrous sodium sulfate. The solvent was evaporated and the residue was purified by column chromatography using 10% ethyl acetate in hexane as eluant to yield the title compound (200 mg).

EIMS (m/z): 258.06 (M+H)

Analogue of 2-(4-bromo-2-fluorophenyl)-5-methyl-1,3,4-oxadiazole as below was prepared by replacing 4-bromo-2-fluorobenzonitrile with appropriate benzonitrile.
2-(4-bromophenyl)-5-methyl-1,3,4-oxadiazole

### Synthesis of 5-bromo-2-[5-(4-fluorophenyl)-1H-tetrazol-1-yl]pyridine

### Step a: Synthesis of N-(5-bromopyridin-2-yl)-4-fluorobenzamide

To a solution of 5-bromo-2-aminopyridine (3 g) in dichloromethane (40 mL) at 0° C. was added triethylamine (3.5 mL). The reaction mixture was stirred for 1 hour followed by the addition of 4-fluorobenzoyl chloride (3.2 mL) and a catalytic amount of 4-dimethylaminopyridine (0.05 g). The reaction was mixture stirred overnight at room temperature and then poured into a solution of sodium hydrogen carbonate and filtered. The organic layer was dried over anhydrous sodium sulfate and concentrated to afford crude product, which was recrystallized with hexane to yield the title compound (4.2 g).

### Step b: Synthesis of 5-bromo-2-[5-(4-fluorophenyl)-1H-tetrazol-1-yl]pyridine

To a compound (4.2 g) obtained from the step a above in toluene (30 mL) was added phosphorous pentachloride (1.5 g) and refluxed for 15 hours. The solvent was evaporated under reduced pressure and the reaction mixture was poured into a precooled solution of acetone (35 mL). A precooled solution of water (25 mL) with sodium azide (1.08 g) and sodium acetate (2.046 g) was added into the solution and the resulting mixture was stirred for 12 hours at room temperature. The solvent was removed and resulting residue extracted with dichloromethane, dried over anhydrous sodium sulfate and the solvent was evaporated. The crude product thus obtained was recrystallized from hexane:diethyl ether mixture to yield the title compound (600 mg)

EIMS (m/z): 321.13 (M+H)

### Synthesis of 5-(5-bromo-2-thienyl)-2-methyl-2H-tetrazole

### Step a: Synthesis of 5-(5-bromo-2-thienyl)-2H-tetrazole

To a solution of 5-bromothiophene-2-carbonitrile (1.5 g) in toluene was added sodium azide (1.33 g) and triethylamine hydrochloride (2.89 g) and the reaction mixture was stirred overnight at 100-110° C. The reaction mixture was filtered and the solid was washed with methanol. The filtrate was concentrated under vacuum to yield the title compound (1.8 g).

### Step b: Synthesis of 5-(4-bromo-2-thienyl)-2-methyl-2H-tetrazole

To a compound (4.2 g) obtained from the step a above in dimethylformamide (30 mL) was added, potassium hydroxide (2.08 g) and methyl iodide (3.5 mL). The reaction mixture was stirred for 12 hours at room temperature. The solvent was removed and the resulting residue extracted with dichloromethane, dried over anhydrous sodium sulfate and the solvent was evaporated. The crude product thus obtained was purified by column chromatography using dichloromethane as eluant to yield the title compound. (1.8 g)

EIMS (m/z): 246.10 (M+H)

### Synthesis of 5-bromo-2-(1H-imidazol-1-yl)pyridine

To a solution of 2,5-dibromopyridine (1.0 g), imidazole (0.574 g) in N-methyl-2-pyrrolidone was added potassium carbonate (1.76 g) at room temperature and the reaction mixture was refluxed overnight at 110-120° C. The reaction mixture was quenched with water (20 mL) and then extracted with ethyl acetate. Volatiles were removed under vacuum and the residue obtained was triturated with water. The solid thus separated out was filtered and dried to yield the title compound (0.8 g).

EIMS (m/z): 225.06 (M+H)

### Synthesis of [1-(5-bromopyridin-2-yl)-1H-imidazol-4-yl]methanol

1-(5-bromopyridin-2-yl)-1H-imidazole-4-carbaldehyde (600 mg) was dissolved in methanol (20 mL) followed by the addition of sodium borohydride (90 mg). The reaction mixture was stirred at room temperature for 2 hours. Volatiles were removed under vacuo and the residue was diluted with dichloromethane (200 mL) and washed with water. The resulting organic layer was dried over anhydrous sodium sulfate and the solvent was removed and triturating over hexane to yield the title compound (400 mg).

EIMS (m/z): 255.09 (M+H)

### Synthesis of 5-bromo-2-(1H-pyrazol-1-yl)pyrimidine

To a solution of 5-bromo-2-chloropyrimidine (560 mg) in N-methylpyrrolidin-2-one (5 mL) was added 1H-pyrazole (200 mg) and potassium carbonate (500 mg). The reaction mixture was stirred at 80° C. for 5 hours, then poured into cooled water (100 mL). The solid that separated out was filtered and dried under high vacuum to yield the title compound. (400 mg)

EIMS (m/z): 226.05 (M+H)

### Synthesis of 5-(5-bromopyridin-2-yl)-1,3,4-thiadiazol-2-amine

To a solution of 5-bromopyridine-2-carbonitrile (2 g) in trifluoroacetic acid (25 mL) was added thiosemicarbazide (0.91 g) and the reaction mixture was refluxed for 15 hours. The solvent was evaporated and the resulting product was taken in ethyl acetate and neutralized with sodium bicarbonate solution. The aqueous layer that separated out was dried with anhydrous sodium sulfate and concentrated to yield the title compound (1.35 g).

EIMS (m/z): 258.1 (M+H)

### Synthesis of 5-(4-bromo-2-furyl)-2-methyl-1H-tetrazole (A) and 5-(4-bromo-2-furyl)-1-methyl-1H-tetrazole (B)

### Step a: Synthesis of 5-(5-bromo-2-furyl)-1H-tetrazole

To a solution of 5-bromo-2-furonitrile (2.5 g) in toluene was added sodium azide (1.33 g) and triethylamine hydrochloride (2.89 g). The reaction mixture was stirred at 100-110° C. overnight. The reaction mixture was filtered and the solid was washed with methanol. The filtrate was concentrated under vacuum to yield the title compound (2.4 g).

### Step b: Synthesis of 5-(4-bromo-2-furyl)-1-methyl-1H-tetrazole

To a compound (2.4 g) obtained from the step a above in dimethylformamide (30 mL) was added potassium hydroxide (2.08 g) and methyl iodide (3.5 mL). The reaction mixture was stirred for 12 hours at room temperature. The solvent was removed and the resulting residue extracted with dichloromethane, dried over anhydrous sodium sulfate and the solvent was evaporated. The crude product thus obtained was purified by column chromatography using dichloromethane as eluant to yield 5-(4-bromo-2-furyl)-2-methyl-1H-tetrazole (1.6 g) (A) and 5-(4-bromo-2-furyl)-1-methyl-1H-tetrazole. (300 mg) (B)

EIMS (m/z): 230.4 (M+H) (A),

EIMS (m/z): 230.32 (M+H) (B).

### Synthesis of 5-bromo-2-(4-phenyl-1H-imidazol-1-yl)pyridine

To a solution of 2,5-dibromopyridine (2 g) in N-methylpyrrolidin-2-one (20 mL) was added 4-phenyl-1H-imidazole (2.4 g) and potassium carbonate (3.5 g). The reaction mixture was stirred overnight at 100-110° C. The reaction mixture was poured into water, extracted with ethyl acetate and washed with brine. The organic layer was dried over anhydrous sodium sulfate and concentrated. The product thus obtained was purified by column chromatography using 50% dichloromethane/Hexane as eluant to afford the title compound (1.2 g).

EIMS (m/z): 300.16 (M+H)

### Synthesis of 5-bromo-2-(1H-1,2,3-triazol-1-yl)pyridine

To a solution of 5-bromo-2-chloropyridine (560 mg) in N-methylpyrrolidin-2-one (5 mL) was added 1H-1,2,3-triazole (200 mg) and potassium carbonate (803 mg). The reaction mixture was stirred at 80° C. for 2 hours and the reaction mixture was poured into cooled water (100 mL). The solid separated out was filtered and dried under high vacuum to yield the title compound. (400 mg)

EIMS (m/z): 226.05 (M+H)

### Scheme 1

### Example 1

### Synthesis of N-({(5S)-3-[3,5-difluoro-4-(trimethylstannyl)phenyl]-2-oxo-1,3-oxazolidin-5-yl}methyl)acetamide

### Step a: Synthesis of (S)-[N-3-(3,5-Difluorophenyl)-2-oxo-5-oxazolidinyl]-methyl acetamide

To a solution of (S)-[N-3-(3,5-difluorophenyl)-2-oxo-5-oxazolidinyl]methyl amine (8.9 g; synthesized following the procedure as per described in WO 93/09103) in dichloromethane at 0 to 5° C. was added triethylamine (5.91 g) and acetic anhydride (4.77 g). The reaction mixture was stirred at room temperature for about 4 hours and diluted with dichloromethane (50 mL), washed with saturated sodium bicarbonate and brine. The organic layer was dried over anhydrous sodium sulfate and the solvent was removed under vacuum. The residue was purified by column chromatography using 2% methanol-dichloromethane as eluant to yield the title compound (8.0 g).

EIMS (m/z): 271 (M+H)

### Step b: Synthesis of (S)-[N-3-(4-iodo-3,5-difluorophenyl)-2-oxo-5-oxazolidinyl]methyl acetamide

To a solution of the compound (7.0 g) obtained from step a above, in chloroform:acetonitrile (3:1) mixture (100 mL) was added silver trifluoroacetate (7.56 g). To the reaction mixture was added iodine (6.58 g) portion wise and the reaction mixture was stirred overnight at room temperature. The reaction mixture was filtered and the solvents were removed under vacuum. The residue was taken in water and filtered to yield the title compound (7.5 g).

EIMS (m/z): 397.1 (M+H)]

### Step c: Synthesis of N-({(5S)-3-[3,5-difluoro-4-(trimethylstannyl)phenyl]-2-oxo-1,3-oxazolidin-5-yl}methyl)acetamide

To a solution of the compound (4.9 g) obtained from the Step b above, in dioxane (100 mL) was added hexamethyldistannane (5 g) and dichorobistriphenylphosphine palladium (II) (2.1 g) and the reaction mixture was stirred at 80° C. for 2 hours. The reaction mixture was filtered and the solvents were removed under vacuum. The crude product was purified by column chromatography using 0.5% methanol-dichloromethane as eluant to yield the title compound (4.5 g).

EIMS (m/z): 433

Analog of N-({(5S)-3-[3,5-difluoro-4-(trimethylstannyl)phenyl]-2-oxo-1,3-oxazolidin-5-yl}methyl)acetamide, described below was prepared by replacing (S)-[N-3-(3,5-difluorophenyl)-2-oxo-5-oxazolidinyl]methylamine with (5S)-5-(aminomethyl)-3-(3-fluorophenyl)-1,3-oxazolidin-2-one:
N-({(5S)-3-[3-fluoro-4-(trimethylstannyl)phenyl]-2-oxo-1,3-oxazolidin-5-yl}methyl)acetamide.

### Scheme II

### Example 2

### Synthesis of tert-butyl {[(5R)-3-(3,5-difluoro-4-iodophenyl)-2-oxo-1,3-oxazolidin-5-yl]methyl}-1,3-thiazol-2-ylcarbamate

### Step a: Synthesis of (5R)-3-(3,5-difluoro-4-iodophenyl)-5-(hydroxymethyl)-1,3-oxazolidin-2-one

To a solution of 3-(3,5-difluoro-4-iodophenyl)-5-(hydroxymethyl)-1,3-oxazolidin-2-one (9.13 g) in acetonitrile (87.5 mL) and dichloromethane (62.5 mL) was added trifluorosilver acetate and the reaction mixture was stirred for 15 minutes followed by slow addition of iodine. The reaction mixture was stirred for 12 hours at room temperature and filtered. The filtrate was concentrated and the slurry was poured into ice-cooled water. The separated precipitate separated out was filtered and dried to yield the title compound (12.6 g).

EIMS (m/z): 356.03

### Step b: Synthesis of [(5R)-3-(3,5-difluoro-4-iodophenyl)-2-oxo-1,3-oxazolidin-5-yl]methyl methanesulfonate

To a solution of the compound (10 g) obtained from the step a above in dichloromethane (150 mL) was added triethylamine (4.5 g). The reaction mixture was cooled to 5° C., followed by dropwise addition of mesylchloride (4.49 g). The resulting reaction mixture was stirred for 2 hours, diluted with dichloromethane, washed with sodium hydrogen carbonate, brine and dried over anhydrous sodium sulfate. The organic layer was concentrated and the crude was recrystallized from hexane to yield the title compound (11.17 g).

EIMS (m/z): 434

Analog of [(5R)-3-(3,5-difluoro-4-iodophenyl)-2-oxo-1,3-oxazolidin-5-yl]methyl methanesulfonate, described below was prepared by replacing (5R)-3-(3,5-difluoro-4-iodophenyl)-5-(hydroxymethyl)-1,3-oxazolidin-2-one with (5R)-3-(3-fluoro-4-iodophenyl)-5-(hydroxymethyl)-1,3-oxazolidin-2-one:
[(5R)-3-(3-fluoro-4-iodophenyl)-2-oxo-1,3-oxazolidin-5-yl]methyl methanesulfonate

### Step c: Synthesis of tert-butyl {[(5R)-3-(3,5-difluoro-4-iodophenyl)-2-oxo-1,3-oxazolidin-5-yl]methyl}1,3-thiazol-2-ylcarbamate

To a solution of the compound (5.5 g) obtained from the step b above in dry dimethylformamide (100 mL) was added sodium hydride (760 mg) and tertbutyl-1,3-thiazole-2-yl-carbamate (2.8 g). The reaction mixture was heated for 1 hour at 80° C., poured into water and extracted with dichloromethane washed with brine and dried over anhydrous sodium sulfate. The solvent was removed under vacuum and the crude product was purified by column using 10% ethyl acetate in hexane as eluant to yield the title compound (4.06 g)

¹HNMR (CDCl₃): δ 7.39-7.38 (d, 1H), 7.26-7.15 (dd, 2H), 6.99-6.97 (d, 1H), 5.14-5.08 (m, 1H), 4.48-4.46 (m, 2H), 4.08 (t, 1H), 3.87-3.85 (m, 1H), 1.59 (s, 9H).

EIMS (m/z): 538 (M+H)

Analog of tert-butyl {[(5R)-3-(3,5-difluoro-4-iodophenyl)-2-oxo-1,3-oxazolidin-5-yl]methyl}1,3-thiazol-2-ylcarbamate described below was prepared by replacing tertbutyl-1,3-thiazole-2-yl-carbamate with tert-butyl isoxazol-3-ylcarbamate
tert-butyl {[(5R)-3-(3,5-difluoro-4-iodophenyl)-2-oxo-1,3-oxazolidin-5-yl]methyl}isoxazol-3-ylcarbamate.

EIMS (m/z): 522 (M+H)

### Scheme III

### Example 3

### Synthesis of 3-Fluoro-4-(hydroxyimino-methyl)-benzene boronic acid

A solution of 4-bromo-2-fluorobenzaldehyde oxime (4 g) and triisopropyl borate (8.5 mL) in tetrahydrofuran was cooled to −78° C. To the solution was added n-butylamine (21 mL) in hexane and the reaction mixture was stirred at −78° C. for about 4 hours. The reaction was quenched with water (10 mL) and was allowed to stir at room temperature for about 12 hours. The solvents were removed under vacuum and the reaction mixture was washed diethyl ether to remove unwanted impurities. Aqueous layer was acidified with 50% aqueous HCl to yield a white precipitate which was filtered and dried to yield the title compound (1.5 g).

EIMS (m/z): 184.38 (M+H)

Analogues of 3-Fluoro-4-(hydroxyimino-methyl)-benzene boronic acid described below were prepared by replacing 4-bromo-2-fluorobenzaldehyde oxime with appropriate oximes or heterocycles as applicable in each case.
[3-fluoro-4-(1,3-oxazol-5-yl)benzene]boronic acid

EIMS (m/z): 208.28 (M+H);
[4-(1H-benzimidazol-2-yl)-3-fluorophenyl]boronic acid
[6-(1H-1,2,4-triazol-1-yl)pyridin-3-yl]boronic acid

EIMS (m/z): 191.39 (M+H);
[5-(1,3-oxazol-5-yl)-2-furyl]boronic acid

EIMS (m/z): 180.38 (M+H);
[5-(1,3-oxazol-5-yl)-2-thienyl]boronic acid

EIMS (m/z): 196.34 (M+H);
[6-(1H-imidazol-2-yl)pyridin-3-yl]boronic acid

EIMS (m/z): 190.35 (M+H);
[6-(3-formyl-1H-pyrrol-1-yl)pyridin-3-yl]boronic acid

EIMS (m/z): 217.19 (M+H);
(6-{3-[(Z)-(hydroxyimino) methyl]-1H-pyrrol-1-yl}pyridin-3-yl) boronic acid

EIMS (m/z): 232.23 (M+H);
[6-(1-methyl-1H-tetrazol-5-yl)pyridin-3-yl]boronic acid

EIMS (m/z): 206 (M+H);
[6-(2-methyl-1H-tetrazol-5-yl)pyridin-3-yl]boronic acid

EIMS (m/z): 206 (M+H);
[6-(5-methyl-1,3,4-oxadiazol-2-yl)pyridin-3-yl]boronic acid

EIMS (m/z): 206 (M+H);
[3-fluoro-4-(5-methyl-1H-tetrazol-1-yl)phenyl]boronic acid

EIMS (m/z): 223 (M+H);
[3-fluoro-4-(5-phenyl-1H-tetrazol-1-yl)phenyl]boronic acid

EIMS (m/z): 286 (M+H);
[6-(5-phenyl-1H-tetrazol-1-yl)pyridin-3-yl]boronic acid

EIMS (m/z): 268 (M+H);
[6-(4-pyridin-3-yl-1H-imidazol-1-yl)pyridin-3-yl]boronic acid.

EIMS (m/z): 267 (M+H).

### Scheme IV

### Example 4

### Synthesis of N-[((5S)-3-{3,5-difluoro-4-[6-(3-formyl-1H-pyrrol-1-yl)pyridin-3-yl]phenyl}-2-oxo-1,3-oxazolidin-5-yl)methyl]acetamide (Compound No. 1)

### Step a: Synthesis of tert-butyl (5-bromopyridin-2-yl) carbamate

A solution of 5-bromopyridin-2-amine (3 g) in dichloromethane (80 mL) was cooled to 0° C. and to it was added triethylamine (3.8 g), di-text-butyl dicarbonate (4.89 g) and 4-dimethylaminopyridine (150 mg). The reaction mixture was stirred at room temperature for 2-3 hours and diluted with dichloromethane. The organic layer was washed with a saturated solution of sodium hydrogen carbonate and brine solution and dried over anhydrous sodium sulfate. The solvent was concentrated to form the crude product which was recrystallized with hexane to yield the title compound (2.2 g).

### Step b: Synthesis of {6-[(tert-butoxycarbonyl)amino]pyridin-3-yl}boronic acid

To the compound (2.0 g) obtained from the step a above in tetrahydrofuran (40 mL) was added triisopropylborate (4.25 mL) and the reaction mixture was stirred under argon atmosphere. The reaction mixture was cooled to −78° C. and to it butyl lithium was added dropwise. The reaction mixture was stirred at −78° C. for 4 hours and quenched with water (10 mL) and concentrated. The residue was washed with ether and acidified with 30% aqueous hydrochloride to pH 5. The solid was filtered to yield the title product (1.05 g).

### Step c: Synthesis of tert-butyl [5-(4-{(5R)-5-[(acetyl amino)methyl]-2-oxo-1,3-oxazolidin-3-yl}-2,6-difluorophenyl)pyridin-2-yl]carbamate

To the compound (360 mg) obtained from the step b above in 1-propanol (40 mL) was added (S)-[N-3-(4-iodo-3,5-difluorophenyl)-2-oxo-5-oxazolidinyl]methyl acetamide (600 mg) obtained from step b of Example 1, Scheme I. The reaction mixture was stirred under argon temperature for 10 minutes, followed by the addition of palladium diacteate (56.6 mg) and triphenylphosphine (198.7 mg) and then stirred for an additional 10 minutes. To this was added sodium carbonate (133.8 mg) (dissolved in water) and the reaction mixture was degassed. The reaction mixture was heated for 1 hour at 100-110° C. and quenched with water:ethyl acetate (15:100 mL). The organic layer was washed with a saturated solution of sodium hydrogen carbonate and brine, dried over anhydrous sodium sulfate and concentrated to form a crude compound, which was purified by column chromatography using 1-3% methanol in dichloromethane to yield the title compound (170 mg).

### Step d: Synthesis of N-({(5S)-3-[4-(6-aminopyridin-3-yl)-3,5-difluorophenyl]-2-oxo-1,3-oxazolidin-5-yl}methyl)acetamide

The compound (170 mg) obtained from step c above was taken in ethanol and to it was added 3N hydrochloric acid. The reaction mixture was stirred at room temperature for an hour and the solvent was evaporated to yield title compound (160 mg).

### Step e: Synthesis of N-[((5S)-3-{3,5-difluoro-4-[6-(3-formyl-1H-pyrrol-1-yl)pyridin-3-yl]phenyl}-2-oxo-1,3-oxazolidin-5-yl)methyl]acetamide

To the compound (160 mg) obtained from the step d above in acetic acid (7 mL) was added 2,5-dimethoxytetrahydrofuran-3-carbaldehyde (247 mg) and the reaction mixture was stirred for 2-3 hours at 110-120° C. The solvent was evaporated and the resulting product was taken in dichloromethane, washed with brine and dried over anhydrous sodium sulfate. The resulting organic layer was concentrated to form the crude compound, which was purified by column chromatography using 10% methanol in dichloromethane as eluant to yield the title compound (88 mg).

¹HNMR (CDCl₃): δ 9.85 (s, 1H), 8.61 (s, 2H), 8.29-7.88 (m, 4H), 7.49 (m, 2H), 6.74 (s, 1H), 4.81 (m, 1H), 4.22 (m, 1H), 3.79 (m, 1H), 3.47 (m, 2H), 1.85 (s, 3H);

EIMS (m/z) 441.25 (M+H)

### Example 5

### Synthesis of N-{[(5S)-3-[3,5-difluoro-4-{6-[3-(hydroxymethyl)-1H-pyrrol-1-yl]pyridin-3-yl}phenyl)-2-oxo-1,3-oxazolidin-5-yl]methyl}acetamide (Compound No. 2)

The compound (80 mg) obtained from the step e of the Example 4, in dichloromethane (2 mL):methanol (4 mL) was cooled to 0° C. To the reaction mixture was added sodium borohydride (30.8 mg) portion wise at room temperature. The reaction mixture was stirred for 6 hours, diluted with dichloromethane and treated with saturated solution of ammonium chloride. The resulting organic layer was separated, dried over anhydrous sodium sulfate, concentrated and purified by column chromatography using methanol-dichloromethane as eluant to yield the title compound (25 mg).

¹HNMR (DMSO-d6): δ 8.49 (s, 1H), 8.29 (m, 1H), 7.81 (m, 1H), 7.78 (m, 1H), 7.66 (m, 2H), 7.50 (m, 2H), 6.30 (bs, 1H), 4.80 (m, 1H), 4.77 (s, 2H), 4.21 (t, 1H), 3.78 (t, 1H), 3.37 (m, 2H), 1.85 (s, 3H);

EIMS (m/z) 443.16 (M+H)

### Example 6

### Synthesis of N-({(5S)-3-[3-fluoro-4-(2-{3-[(E)-(hydroxyimino)methyl]-1H-pyrrol-1-yl}pyrimidin-5-yl)phenyl]-2-oxo-1,3-oxazolidin-5-yl}methyl)acetamide (Compound No. 3)

### Step a: Synthesis of (5-bromopyrimidin-2-yl)tert-butylcarbamate

A solution 5-bromopyrimidin-2-amine (3 g) in dichloromethane (80 mL) was cooled to 0° C. To it was added triethylamine (3.8 g), di-tert-butyl dicarbonate (4.89 g) and 4-dimethylaminopyridine (150 mg). The reaction mixture was stirred at room temperature for 2-3 hours, diluted with dichloromethane. The organic layer was washed with saturated solution of sodium hydrogen carbonate and brine solution and dried over anhydrous sodium sulfate. The solvent was concentrated to give the crude product, which was recrystallized with hexane to yield the title compound (2.2 g).

### Step b: Synthesis of {2-[tert-butoxycarbonyl)amino]pyrimidin-5-yl}boronic acid

To the compound (2.0 g) obtained from the step a above in tetrahydrofuran (40 mL) was added triisopropylborate (4.25 mL) and the reaction mixture was stirred under argon atmosphere. The reaction mixture was cooled to −78° C. and to it was added butyl lithium dropwise. The reaction mixture was stirred at −78° C. for 4 hours and quenched with water (10 mL) and concentrated. The residue was washed with ether and acidified with 30% aqueous hydrochloride to pH 5. The solid was filtered to yield the title product (1.05 g).

### Step c: Synthesis of tert-butyl [5-(4-{(5R)-5-[acetylamino)methyl]-2-oxo-1,3-oxazolidin-3-yl}-2-fluorophenyl)pyrimidin-2-yl]carbamate

To the compound (360 mg) obtained from the step b above in 1-propanol (40 mL) was added N-{[(56)-3-(3-fluoro-4-iodophenyl)-2-oxo-1,3-oxazolidin-5-yl]methyl}acetamide (600 mg) obtained from step b of Example 1, Scheme I. The reaction mixture was stirred under argon temperature for 10 minutes, followed by the addition of palladium diacetate (56.6 mg) and triphenyl phosphine (198.7 mg). The reaction mixture was then stirred for 10 minutes. To this was added sodium carbonate (133.8 mg) (dissolved in water) and the reaction mixture was degassed. The reaction mixture was heated for 1 hour at 100-110° C. and quenched with water:ethyl acetate (15:100 mL). The organic layer was washed with saturated solution of sodium hydrogen carbonate and brine, dried over anhydrous sodium sulfate and concentrated to form the crude compound, which was purified by column chromatography using 1-3% methanol in dichloromethane to yield the title compound (170 mg).

### Step d: Synthesis of N-({(5S)-3-[4-(2-aminopyrimidin-5-yl)-3-fluorophenyl]-2-oxo-1,3-oxazolidin-5-yl}methyl)acetamide

The compound (170 mg) obtained from step c above was taken in ethanol and to it was added 3N hydrochloric acid. The reaction mixture was stirred at room temperature for an hour and the solvent was evaporated to yield title compound (160 mg).

### Step e: Synthesis of N-[((5S)-3-{3-fluoro-4-[2-(3-formyl-1H-pyrrol-1-yl)pyrimidin-5-yl]phenyl}-2-oxo-1,3-oxazolidin-5-yl)methyl]acetamide

To the compound (160 mg) obtained from the step d above in acetic acid (7 mL) was added 2,5-dimethoxytetrahydrofuran-3-carbaldehyde (247 mg) and the reaction mixture was stirred for 2-3 hours at 110-120° C. The solvent was evaporated and the compound was taken in dichloromethane, washed with brine and dried over anhydrous sodium sulfate. The organic layer was concentrated to form a crude compound, which was purified by column chromatography using 10% methanol in dichloromethane as eluant to yield the title compound (88 mg).

### Step f: Synthesis of N-({(5S)-3-[3-fluoro-4-(2-{3-[(E)-(hydroxyimino)methyl]-1H-pyrrol-1-yl}pyrimidin-5-yl)phenyl]-2-oxo-1,3-oxazolidin-5-yl}methyl)acetamide

To a solution of N-[((5S)-3-{3-fluoro-4-[2-(3-formyl-1H-pyrrol-1-yl)pyrimidin-5-yl]phenyl}-2-oxo-1,3-oxazolidin-5-yl)methyl]acetamide (200 mg) in methanol (5 mL) was added hydroxylamine hydrochloride (50 mg) and the reaction mixture was stirred at 25° C. for about 15 hours. Volatiles were removed under vacuum and the crude product was purified by column chromatography using 5% methanol in dichloromethane as eluant. (30 mg).

¹HNMR (DMSOd6): δ 11.34 (s, 1H), 9.03 (s, 2H), 8.47 (m, 1H), 8.10 (s, 1H), 7.79 (m, 2H), 7.78 (d, 1H), 7.50 (m, 1H), 4.78 (m, 1H), 4.19 (t, 1H), 3.80 (t, 1H), 3.47 (m, 2H), 1.85 (s, 3H);

EIMS (m/z): 439.12 (M+H)

Analogue of N-({(5S)-3-[3-fluoro-4-(2-{3-[(E)-(hydroxyimino)methyl]-1H-pyrrol-1-yl}pyrimidin-5-yl)phenyl]-2-oxo-1,3-oxazolidin-5-yl}methyl)acetamide as given below can be prepared by replacing N-{[(5S)-3-(3-fluoro-4-iodophenyl)-2-oxo-1,3-oxazolidin-5-yl]methyl}acetamide with appropriate acetamide:
N-({(5S)-3-[3,5-difluoro-4-(2-{3-[(E)-(hydroxyimino)methyl]-1H-pyrrol-1-yl}pyrimidin-5-yl)phenyl]-2-oxo-1,3-oxazolidin-5-yl}methyl)acetamide (Compound no: 4),

EIMS (m/z): 457.09 (M+H)
N-{[(5S)-3-(2,3′-difluoro-4′-{3-[(E)-(hydroxyimino)methyl]-1H-pyrrol-1-yl}biphenyl-4-yl)-2-oxo-1,3-oxazolidin-5-yl]methyl}acetamide (Compound No. 24),

EIMS (m/z) 455.06 (M+H);

### Scheme V

### Example 7

### Synthesis of N-({(5S)-3-[4′-((E)-{[(3,4-difluorobenzyl)oxy]imino}methyl)-2,3′,6-trifluorobiphenyl-4-yl]-2-oxo-1,3-oxazolidin-5-yl}methyl)acetamide (Compound No. 5)

### Step a: Synthesis of 4-bromo-2-fluorobenzaldehyde oxime

To a solution of 4-bromo-2-fluorobenzaldehyde (4.04 g) in ethanol (50 mL) was added hydroxylamine hydrochloride (2.07 g). The reaction mixture was stirred at room temperature for 15 hours, water was added and the reaction mixture was stirred for an additional 1 hour. White precipitate that separated out was filtered to yield the title compound (4 g).

### Step b: Synthesis of (3-fluoro-4-hydroxyiminomethyl)phenyl boronic acid

To a solution of the compound (4 g) obtained from the step a above in tetrahydrofuran (100 mL) was added triisopropyl borate (8.55 mL) at −78° C. The reaction mixture was stirred for 10-15 minutes, n-butyl lithium (21 mL) was added and the reaction mixture was further stirred at −78° C. for 4 hours. The reaction mixture was quenched with water and stirred at room temperature overnight followed by extraction with diethylether to remove impurities. The water layer was acidified with aqueous hydrochloric acid (50 mL), extracted with ethyl acetate and concentrated to yield the title compound (1.5 g).

### Step c: Synthesis of N-[((5S)-2-oxo-3-{2,3′,6-trifluoro-4′-[(E)-(hydroxyimino) methyl]biphenyl-4-yl}-1,3-oxazolidin-5-yl)methyl]acetamide

To the compound obtained from the step b above (360 mg) in n-propanol (30 mL) was added (S)-[N-3-(4-iodo-3,5-difluorophenyl)-2-oxo-5-oxazolidinyl]methyl acetamide (600 mg) obtained from step b of Example 1 (Scheme I). The reaction mixture was stirred under argon at room temperature for 10 minutes followed by the addition of palladium diacetate (44 mg) and triphenylphosphine (160 mg). The reaction mixture was stirred for another 10 minutes followed by the addition of sodium carbonate (240 mg) (dissolved in water) and the reaction mixture was then degassed. The reaction mixture was heated for 1 hour at 110° C. and quenched with water ethyl acetate mixture. The organic layer was washed with saturated solution of sodium hydrogen carbonate and brine, dried over anhydrous sodium sulfate and concentrated to form the crude compound, which was purified by column chromatography using 1-3% methanol in dichloromethane to yield the title compound (300 mg).

### Step d: N-({(5S)-3-[4′-((E)-{[(3,4-difluorobenzyl)oxy]imino}methyl)-2,3′,6-trifluorobiphenyl-4-yl]-2-oxo-1,3-oxazolidin-5-yl}methyl)acetamide

To the compound obtained from the step c above (75 mg) in tetrahydrofuran (20 mL) was added, potassium hydroxide (56 mg), tetrabutylammonium iodide (37 mg) and 3,4-difluorobenzylbromide (206 mg). The reaction mixture was stirred at room temperature for 15 hours. Solvents were removed under vacuum and the resulting product was dissolved in ethyl acetate (100 mL) and washed with water. The solvent was removed and the resulting crude compound was purified by column chromatography using 1-3% methanol dichloromethane to yield the title compound (25 mg).

¹HNMR (CDCl₃): δ 8.39 (s, 1H), 7.85 (t, 1H), 7.22 (m, 7H), 6.06 (bs, 1H), 5.17 (s, 2H), 4.83 (m, 1H), 4.06 (m, 1H), 3.81 (m, 1H), 3.69 (m, 2H), 2.04 (s, 3H);

EIMS (m/z) 534.33 (M+H)

### Example 8

### Synthesis of N-{[(5S)-3-(4′-{(E)-[(acetyloxy)imino]methyl}-2,3′,6-trifluorobiphenyl-4-yl)-2-oxo-1,3-oxazolidin-5-yl]methyl}acetamide (Compound No. 6)

To the compound (65 mg) obtained from the step c of Example 7 in dichloromethane (25 mL) was added triethylamine (0.077 mL) and acetyl chloride (0.047 mL). The resulting reaction mixture was stirred for 12 hours. The reaction mixture was poured into water and extracted with dichloromethane, washed with brine and dried over anhydrous sodium sulfate. Solvent was evaporated and the crude was purified by column chromatography using 1-3% methanol in dichloromethane to yield the title compound (44 mg).

¹HNMR (CDCl₃): δ 8.67 (s, 1H), 7.05 (t, 1H), 7.28 (m, 4H), 6.22 (bs, 1H), 4.85 (m, 1H), 4.08 (t, 1H), 3.83 (t, 1H), 3.71 (m, 2H), 2.27 (s, 3H), 2.14 (s, 3H);

EIMS (m/z) 450.21 (M+H)

Analogues of N-{[(5S)-3-(4′-{(E)-[(acetyloxy)imino]methyl}-2,3′,6-trifluorobiphenyl-4-yl)-2-oxo-1,3-oxazolidin-5-yl]methyl}acetamide described below were prepared by replacing acetyl chloride with appropriate acylating or sulfonating agents as applicable in each case:
N-{[(5S)-3-(4′-{(E)-[(benzoyloxy)imino]methyl}-2,3′,6-trifluorobiphenyl-4-yl)-2-oxo-1,3-oxazolidin-5-yl]methyl}acetamide (Compound No. 7),

EIMS (m/z) 512.22 (M+H);
N-({(5S)-2-oxo-3-[2,3′,6-trifluoro-4′-((E)-{[(methylsulfonyl)oxy]imino}methyl)biphenyl-4-yl]-1,3-oxazolidin-5-yl}methyl)acetamide (Compound No. 8)

EIMS (m/z) 486.22 (M+H).

### Example 9

### Synthesis of N-({(5S)-2-oxo-3-[2,3′,6-trifluoro-4′-((E)-{[({[4-(trifluoromethyl)phenyl]amino}carbonyl)oxy]imino}-methyl)biphenyl-4-yl]-1,3-oxazolidin-5-yl}methyl)acetamide (Compound No. 9)

To the compound (60 mg) obtained from the step c of Example 7 in tetrahydrofuran (20 mL) was added sodium hydride (4.7 mg) and 4-trifluoromethyl phenyl isocyanate (0.047 mL). The reaction mixture was then stirred for 3-4 hours at room temperature. The reaction mixture was quenched with ammonium chloride solution, the organic layer was separated and concentrated to form crude compound, which was purified by column chromatography using 1% methanol in dichloromethane to yield the title compound (80 mg).

¹HNMR (DMSO-d6): δ 10.43 (s, 1H), 8.76 (s, 1H), 8.30-8.25 (m, 1H), 8.06 (t, 1H), 7.78-7.45 (m, 4H), 4.82-4.78 (m, 1H), 4.18 (t, 1H), 3.80 (t, 1H), 3.50 (m, 2H), 1.86 (s, 3H).

EIMS (m/z) 595.25 (M+H)

Analogues of N-({(5S)-2-oxo-3-[2,3′,6-trifluoro-4′-((E)-{[({[4-(trifluoromethyl)phenyl]amino}carbonyl)oxy]imino}methyl)biphenyl-4-yl]-1,3-oxazolidin-5-yl}methyl)acetamide described below were prepared by replacing 4-trifluoromethyl phenyl isocyanate with appropriate isocyanate as applicable in each case:
N-[((5S)-3-{4′-[(E)-({[(tert-butylamino)carbonyl]oxy}imino)methyl]-2,3′,6-trifluorobiphenyl-4-yl}-2-oxo-1,3-oxazolidin-5-yl)methyl]acetamide (Compound No. 10),

EIMS (m/z): 507.49 (M+H);
N-{[(5S)-2-oxo-3-(2,3′,6-trifluoro-4′-{(E)-[({[(4-fluorophenyl)amino]carbonyl}oxy) imino]methyl}biphenyl-4-yl)-1,3-oxazolidin-5-yl]methyl}acetamide (Compound No. 11)

EIMS (m/z): 545.19 (M+H).

### Scheme VI

### Path A

### Example 10

### Synthesis of N-({(5S)-2-oxo-3-[2,3′,6-trifluoro-4′-(1,3-oxazol-5-yl)biphenyl-4-yl]-1,3-oxazolidin-5-yl}methyl)acetamide (Compound No. 12)

To a solution of 3-fluoro-4-(hydroxyimino-methyl)-benzene boronic acid (512 mg) (obtained from Scheme III) and (S)-[N-3-(4-iodo-3-fluorophenyl)-2-oxo-5-oxazolidinyl]methyl acetamide (852 mg) (obtained from Scheme I) was added n-propanol. The reaction mixture was degassed with argon for about 15 minutes. To the reaction mixture was added palladium diacetate (101 mg) and triphenyl phosphine (357 mg) and the reaction mixture was stirred at room temperature for 15 minutes. Sodium carbonate (261 mg) dissolved in water (1-2 mL) was added and the reaction mixture was stirred at 100° C. for about 1.5 hours. The reaction mixture was cooled and filtered through a celite pad. The reaction mixture was taken in ethyl acetate (100 mL) and the resulting organic layer was washed with aqueous sodium bicarbonate solution and brine. The mixture was dried over anhydrous sodium sulfate and the solvent was removed under vacuum to leave a crude product, which was triturated over diethyl ether to afford the title compound (750 mg)

¹HNMR (DMSO d6): δ 8.56 (s, 1H), 8.28 (t, 1H), 7.72-7.35 (m, 7H), 4.78 (m, 1H), 4.16 (m, 1H), 3.81 (m, 1H), 3.45 (m, 2H), 1.86 (s, 3H);

EIMS (m/z) 414.38 (M+H)

Analogues of N-({(5S)-2-oxo-3-[2,3′,6-trifluoro-4′-(1,3-oxazol-5-yl)biphenyl-4-yl]-1,3-oxazolidin-5-yl}methyl)acetamide, described below were prepared by replacing 3-fluoro-4-oxazol-5-yl-benzene boronic acid with appropriate boronic acids and acetamides as applicable in each case:
N-[((5S)-3-{3,5-difluoro-4-[6-(1H-1,2,4-triazol-1-yl)pyridin-3-yl]phenyl}-2-oxo-1,3-oxazolidin-5-yl)methyl]acetamide (Compound No. 13),

EIMS (m/z) 415.30 (M+H);
N-{[(5S)-3-(3,5-difluoro-4-{6-[5-(1,3-oxazol-4-yl)-2-furyl]pyridin-3-yl}phenyl)-2-oxo-1,3-oxazolidin-5-yl]methyl}acetamide (Compound No. 14),

EIMS (m/z) 404.38 (M+H);
N-{[(5S)-3-(3,5-difluoro-4-{6-[5-(1,3-oxazol-4-yl)-2-thienyl]pyridin-3-yl}phenyl)-2-oxo-1,3-oxazolidin-5-yl]methyl}acetamide (Compound No. 15),

EIMS (m/z) 420.32 (M+H);
N-{[(5S)-3-(3,5-difluoro-4-{6-[3-(hydroxymethyl)-1H-pyrrol-1-yl]pyridin-3-yl}phenyl)-2-oxo-1,3-oxazolidin-5-yl]methyl}acetamide (Compound No. 16),

EIMS (m/z) 432.35 (M+H);
N-({(5S)-3-[3,5-difluoro-4-(6-{3-[(E)-(hydroxyimino)methyl]-1H-pyrrol-1-yl}pyridin-3-yl)phenyl]-2-oxo-1,3-oxazolidin-5-yl}methyl)acetamide (Compound No. 17),

EIMS (m/z) 456.21 (M+H);
N-({(5S)-3-[2,3′-difluoro-4′-(5-methyl-1,2,4-oxadiazol-3-yl)biphenyl-4-yl]-2-oxo-1,3-oxazolidin-5-yl}methyl)acetamide (Compound No. 18),

EIMS (m/z) 429.18 (M+H);
N-({(5S)-2-oxo-3-[2,3′,6-trifluoro-4′-(5-methyl-1,2,4-oxadiazol-3-yl)biphenyl-4-yl]-1,3-oxazolidin-5-yl}methyl)acetamide (Compound No. 19),

EIMS (m/z) 447.18 (M+H);
N-[((5S)-3-{3,5-difluoro-4-[6-(2-methyl-2H-tetrazol-5-yl)pyridin-3-yl]phenyl}-2-oxo-1,3-oxazolidin-5-yl)methyl]acetamide (Compound No. 20),

EIMS (m/z) 430.25 (M+H);
N-[((5S)-3-{3,5-difluoro-4-[6-(1H-imidazol-1-yl)pyridin-3-yl]phenyl}-2-oxo-1,3-oxazolidin-5-yl)methyl]acetamide (Compound No. 21),

EIMS (m/z) 414.21 (M+H);
N-[((5S)-3-{3,5-difluoro-4-[6-(1-methyl-1H-tetrazol-5-yl)pyridin-3-yl]phenyl}-2-oxo-1,3-oxazolidin-5-yl)methyl]acetamide (Compound No. 22),

EIMS (m/z) 430.05 (M+H);
(5S)-3-{3,5-difluoro-4-[6-(2-methyl-2H-tetrazol-5-yl)pyridin-3-yl]phenyl}-5-[(1,3-thiazol-2-ylamino)methyl]-1,3-oxazolidin-2-one (Compound No. 23),

EIMS (m/z) 471.01 (M+H);
N-({(5S)-3-[2,3′-difluoro-4′-(5-methyl-1H-tetrazol-1-yl)biphenyl-4-yl]-2-oxo-1,3-oxazolidin-5-yl}methyl)acetamide (Compound No. 25),

EIMS (m/z) 429.22 (M+H);
N-((S)-3-{3,5-Difluoro-4-[6-[5-methyl-[1,3,4]oxadiazol-2-yl)-pyridin-3-yl]-phenyl}-2-oxo-oxazolidin-5-ylmethyl)-acetamide (Compound No. 26),

EIMS (m/z) 430.04 (M+H);
N-((S)-3-{4-[6-(5-Amino-[1,3,4]thiadiazol-2-yl)-pyridin-3-yl]-3,5-difluoro-phenyl}-2-oxo-oxazolidin-5-ylmethyl)-acetamide (Compound No. 27),

EIMS (m/z) 447.16 (M+H);
N-({(5S)-3-[4′-(1H-benzimidazol-2-yl)-2,3′,6-trifluorobiphenyl-4-yl]-2-oxo-1,3-oxazolidin-5-yl}methyl)acetamide (Compound No. 28),

EIMS (m/z) 482.06 (M+H);
N-[((5S)-3-{3,5-difluoro-4-[6-(1H-1,2,3-triazol-1-yl)pyridin-3-yl]phenyl}-2-oxo-1,3-oxazolidin-5-yl)methyl]acetamide (Compound No. 29),

EIMS (m/z) 415.17 (M+H);
N-[((5S)-3-{3,5-difluoro-4-[6-(4-phenyl-1H-imidazol-1-yl)pyridin-3-yl]phenyl}-2-oxo-1,3-oxazolidin-5-yl)methyl]acetamide (Compound No. 30),

EIMS (m/z) 490.13 (M+H);
N-[((5S)-3-{3-fluoro-4-[5-(2-methyl-2H-tetrazol-5-yl)-2-furyl]phenyl}-2-oxo-1,3-oxazolidin-5-yl)methyl]acetamide (Compound No. 31),

EIMS (m/z) 401.13 (M+H);
N-[((5S)-3-{3-fluoro-4-[5-(1-methyl-1H-tetrazol-5-yl)-2-furyl]phenyl}-2-oxo-1,3-oxazolidin-5-yl)methyl]acetamide (Compound No. 32),

EIMS (m/z) 401.13 (M+H);
N-[((5S)-3-{3,5-difluoro-4-[5-(3-methyl-2,3-dihydro-1H-tetrazol-5-yl)-2-furyl]phenyl}-2-oxo-1,3-oxazolidin-5-yl)methyl]acetamide (Compound No. 33),

EIMS (ink) 419.10 (M+H).

### PATH B

### Example 11

### Synthesis of N-({(5S)-2-oxo-3-[2,3′,6-trifluoro-4′-(1-methyl-1H-benzimidazol-2-yl)biphenyl-4-yl]-1,3-oxazolidin-5-yl}methyl)acetamide (Compound No. 34)

### Step a: Synthesis of 2-(4-bromo-2-fluorophenyl)-1-methyl-1H-benzimidazole

To a solution of 2-(4-bromo-2-fluorophenyl)-1H-benzimidazole (200 mg) in dimethylformamide (10 mL) was added potassium hydroxide (55 mg) and methyl iodide (0.98 mg). The reaction mixture was stirred at room temperature for 17 hours, then taken into ethyl acetate and washed with water. The organic layer was dried over anhydrous sodium sulfate and the solvent was evaporated to form the crude compound, which was purified by column chromatography using 7% ethyl acetate in hexane to yield the title compound (194 mg).

### Step b: Synthesis of N-({(5S)-2-oxo-3-[2,3′,6-trifluoro-4′-(1-methyl-1H-benzimidazol-2-yl)biphenyl-4-yl]-1,3-oxazolidin-5-yl}methyl)acetamide

The compounds obtained from the step a (190 mg) above and step c (160 mg) of Example 1 were taken together in dimethylformamide (20 mL) and to it was added triethylamine (0.106 g) and dichlorobistriphenylphosphine palladium (II)(150 mg). The reaction mixture was heated at 100° C. for 8 hours. The reaction mixture was then concentrated and the resulting crude product was purified by column using 2% methanol in dichloromethane to yield the title compound (33 mg).

¹HNMR (DMSO d6): δ 8.26 (m, 1H), 7.84 (m, 1H), 7.35 (m, 8H), 4.80 (m, 1H), 4.16 (m, 1H), 3.83 (s, 3H), 3.45 (m, 3H), 1.85 (s, 3H);

EIMS (m/z) 495.23 (M+H)

### Example 12

### Synthesis of N-[((5S)-3-{3-fluoro-4-[2-(1H-1,2,4-triazol-1-yl)pyrimidin-5-yl]phenyl}-2-oxo-1,3-oxazolidin-5-yl)methyl]acetamide (Compound No. 51)

5-bromo-2-(1H-1,2,4-triazol-1-yl)pyrimidine (3 g) and the compound obtained form step c of example 1 above (4.6 g) were taken together in dimethylformamide (50 mL) and to it was added triethylamine (1.5 mL) and dichlorobistriphenylphosphine palladium (II) (3.08 g). The reaction mixture was heated at 100° C. for 4 hours. The reaction mixture was filtered over celite and the filtrate was concentrated under high vacuum. The resulting crude product was purified by column using 2% methanol in dichloromethane to yield the title compound (1 g).

¹HNMR (DMSO d6): δ 9.49 (s, 1H), 9.16 (s, 2H), 8.35 (s, 1H), 8.28 (bs, 1H), 7.82 (t, 1H), 7.68 (d, 1H), 7.52 (d, 1H), 4.79 (m, 1H), 4.20 (t, 1H), 3.84 (t, 1H), 3.47 (m, 2H), 1.85 (s, 3H).

EIMS (m/z) 398.18 (M+H);

Analogues of N-({(5S)-2-oxo-3-[2,3′,6-trifluoro-4′-(1-methyl-1H-benzimidazol-2-yl)biphenyl-4-yl]-1,3-oxazolidin-5-yl}methyl)acetamide or N-[((5S)-3-{3-fluoro-4-[2-(1H-1,2,4-triazol-1-yl)pyrimidin-5-yl]phenyl}-2-oxo-1,3-oxazolidin-5-yl)methyl]acetamide described below were prepared by replacing 2-(4-bromo-2-fluorophenyl)-1-methyl-1H-benzimidazole or 5-bromo-2-(1H-1,2,4-triazol-1-yl)pyrimidine with appropriate heteroaryl groups and acetamide as applicable in each case:
N-[5-(4′-{(5S)-5-[(acetylamino)methyl]-2-oxo-1,3-oxazolidin-3-yl}-2′,3,6′-trifluorobiphenyl-4-yl)-1,3,4-thiadiazol-2-yl]acetamide (Compound No. 35),

EIMS (m/z) 506.16 (M+H);
N-({(5S)-3-[2,3′-difluoro-4′-(1,3-thiazol-2-yl)biphenyl-4-yl]-2-oxo-1,3-oxazolidin-5-yl}methyl)acetamide (Compound No. 36),

EIMS (m/z) 430.09 (M+H);
N-[(3-{2,3′-difluoro-4′-[5-(hydroxymethyl)-4,5-dihydroisoxazol-3-yl]biphenyl-4-yl}-2-oxo-1,3-oxazolidin-5-yl)methyl]acetamide (Compound No. 37),

EIMS (m/z) 446.23 (M+H);
N-[((5S)-{3-[3-fluoro-4-[2-(1H-imidazol-1-yl)pyrimidin-5-yl]phenyl}-2-oxo-1,3-oxazolidin-5-yl)methyl]acetamide (Compound No. 38),

EIMS (m/z) 397.15 (M+H);
N-({(5S)-2-oxo-3-[2,3′,6-trifluoro-4′-(1,3-thiazol-2-yl)biphenyl-4-yl]-1,3-oxazolidin-5-yl}methyl)acetamide (Compound No. 39),

EIMS (m/z) 448.09 (M+H);
N-[((5S)-2-oxo-3-{2,3′,6-trifluoro-4′-[(5S)-5-(hydroxymethyl)-2-oxo-1,3-oxazolidin-3-yl]biphenyl-4-yl}-1,3-oxazolidin-5-yl)methyl]acetamide (Compound No. 40),

EIMS (m/z) 480.13 (M+H);
N-({(5S)-3-[2,3′-difluoro-4′-(5-phenyl-1H-tetrazol-1-yl)biphenyl-4-yl]-2-oxo-1,3-oxazolidin-5-yl}methyl)acetamide (Compound No. 41),

EIMS (m/z) 491.75 (M+H);
N-[(3-{3-fluoro-4-[6-(5-phenyl-1H-tetrazol-1-yl)pyridin-3-yl]phenyl}-2-oxo-1,3-oxazolidin-5-yl)methyl]acetamide (Compound No. 42),

EIMS (m/z) 474.12 (M+H);
N-[((5S)-2-oxo-3-{2,3′,6-trifluoro-4′-[5-(hydroxymethyl)isoxazol-3-yl]biphenyl-4-yl}-1,3-oxazolidin-5-yl)methyl]acetamide (Compound No. 43),

EIMS (m/z) 462.15 (M+H);
N-[((5S)-3-{2,3′-difluoro-4′-[5-(hydroxymethyl)isoxazol-3-yl]biphenyl-4-yl}-2-oxo-1,3-oxazolidin-5-yl)methyl]acetamide (Compound No. 44),

EIMS (m/z) 444.06 (M+H);
N-[((5S)-3-{3,5-difluoro-4-[6-(4-pyridin-3-yl-1H-imidazol-1-yl)pyridin-3-yl]phenyl}-2-oxo-1,3-oxazolidin-5-yl)methyl]acetamide (Compound No. 45),

EIMS (m/z) 491.18 (M+H);
N-[((5S)-3-{3,5-difluoro-4-[6-(5-phenyl-1H-tetrazol-1-yl)pyridin-3-yl]phenyl}-2-oxo-1,3-oxazolidin-5-yl)methyl]acetamide (Compound No. 46),

EIMS (m/z) 492.16 (M+H);
N-[((5S)-3-{4-[2-(1H-benzimidazol-1-yl)pyrimidin-5-yl]-3,5-difluorophenyl}-2-oxo-1,3-oxazolidin-5-yl)methyl]acetamide (Compound No. 47),

EIMS (m/z) 465.19 (M+H);
N-[((5S)-3-{4-[2-(1H-benzimidazol-1-yl)pyrimidin-5-yl]-3-fluorophenyl}-2-oxo-1,3-oxazolidin-5-yl)methyl]acetamide (Compound No. 48),

EIMS (m/z) 447.18 (M+H);
N-[((5S)-3-{3,5-difluoro-4-[2-(1H-1,2,4-triazol-1-yl)pyrimidin-5-yl]phenyl}-2-oxo-1,3-oxazolidin-5-yl)methyl]acetamide (Compound No. 49)

EIMS (m/z) 416.18 (M+H);
N-[((5S)-3-{3-fluoro-4-[2-(4-phenyl-1H-imidazol-1-yl)pyrimidin-5-yl]phenyl}-2-oxo-1,3-oxazolidin-5-yl)methyl]acetamide (Compound No. 50),

EIMS (m/z) 473.19 (M+H);
N-({(5S)-2-oxo-3-[2,3′,6-trifluoro-4′-(5-phenyl-1H-tetrazol-1-yl)biphenyl-4-yl]-1,3-oxazolidin-5-yl}methyl)acetamide (Compound No. 52),

EIMS (m/z) 509 (M+H);
N-[((5S)-3-{3,5-difluoro-4-[2-(4-phenyl-1H-imidazol-1-yl)pyrimidin-5-yl]phenyl}-2-oxo-1,3-oxazolidin-5-yl)methyl]acetamide (Compound No. 53),

EIMS (m/z) 491.13 (M+H)
N-[((5S)-3-{3,5-difluoro-4-[2-(2-oxo-1,3-oxazolidin-3-yl)pyrimidin-5-yl]phenyl}-2-oxo-1,3-oxazolidin-5-yl)methyl]acetamide (Compound No. 54),

EIMS (m/z): 433.9 (M+H)
N-[((5S)-3-{3-fluoro-4-[2-(1H-1,2,3-triazol-1-yl)pyrimidin-5-yl]phenyl}-2-oxo-1,3-oxazolidin-5-yl)methyl]acetamide (Compound No. 55),

EIMS (m/z): 398.1 (M+H)
N-[((5S)-3-{4-[2-(3,5-dimethyl-1H-pyrazol-1-yl)pyrimidin-5-yl]-3-fluorophenyl}-1,3-oxazolidin-5-yl)methyl]acetamide (Compound No. 56),

EIMS (m/z): 425.0 (M+H)
N-[((5S)-3-{3-fluoro-4-[2-(2-oxo-1,3-oxazolidin-3-yl)pyrimidin-5-yl]phenyl}-2-oxo-1,3-oxazolidin-5-yl)methyl]acetamide (Compound No. 57),

EIMS (m/z): 416.0 (M+H)
N-{[(5S)-3-(4-{6-[4-(difluoromethyl)-1H-imidazol-1-yl]pyridin-3-yl}-3,5-difluorophenyl)-2-oxo-1,3-oxazolidin-5-yl]methyl}acetamide (Compound No. 58),

EIMS (m/z): 464.08 (M+H)
N-[((5S)-3-{3-fluoro-4-[2-(3-formyl-1H-pyrrol-1-yl)pyrimidin-5-yl]phenyl}-2-oxo-1,3-oxazolidin-5-yl)methyl]acetamide (Compound No. 59),

EIMS (m/z): 424.11 (M+H)
N-({(5S)-2-oxo-3-[2,3′,6-trifluoro-4′-(1H-1,2,4-triazol-1-yl)biphenyl-4-yl]-1,3-oxazolidin-5-yl}methyl)acetamide (Compound No. 60),

EIMS (m/z): 432.04 (M+H)
N-[((5S)-3-{3,5-difluoro-4-[2-(3-formyl-1H-pyrrol-1-yl)pyrimidin-5-yl]phenyl}-2-oxo-1,3-oxazolidin-5-yl)methyl]acetamide (Compound No. 61),

EIMS (m/z): 442.11 (M+H)
N-[((5S)-2-oxo-3-{2,3′,6-trifluoro-4′-[5-(hydroxymethyl)-4,5-dihydroisoxazol-3-yl]biphenyl-4-yl}-1,3-oxazolidin-5-yl)methyl]acetamide (Compound No. 62),

EIMS (m/z): 464.08 (M+H)
N-[((5S)-{3-fluoro-4-[2-(1H-pyrrol-1-yl)pyrimidin-5-yl]phenyl}-2-oxo-1,3-oxazolidin-5-yl)methyl]acetamide (Compound No. 63),

EIMS (m/z): 396.09 (M+H)
N-({(5S)-3-[2,3′-difluoro-4′-(1H-1,2,4-triazol-1-yl)biphenyl-4-yl]-2-oxo-1,3-oxazolidin-5-yl}methyl)acetamide (Compound No. 64),

EIMS (m/z): 414.06 (M+H)
N-({(5S)-3-[3,5-difluoro-4-(6-{4-[(E)-(methoxyimino)methyl]-1H-imidazol-1-yl}pyridin-3-yl)phenyl]-2-oxo-1,3-oxazolidin-5-yl}methyl)acetamide (Compound No. 65),

EIMS (m/z): 471.08 (M+H)
N-[((5S)-3-{3,5-difluoro-4-[6-(4-formyl-1H-imidazol-1-yl)pyridin-3-yl]phenyl}-2-oxo-1,3-oxazolidin-5-yl)methyl]acetamide (Compound No. 66),

EIMS (m/z): 442.01 (M+H)
N-[((5S)-3-{4-[6-(4-cyano-1H-imidazol-1-yl)pyridin-3-yl]-3,5-difluorophenyl}-2-oxo-1,3-oxazolidin-5-yl)methyl]acetamide (Compound No. 67),

EIMS (m/z): 439.06 (M+H)
methyl 1-[5-(4-{(5S)-5-[(acetylamino)methyl]-2-oxo-1,3-oxazolidin-3-yl}-2,6-difluorophenyl)pyridin-2-yl]-1H-imidazole-4-carboxylate (Compound No. 68),

EIMS (m/z): 472.06 (M+H)
N-({(5S)-3-[3,5-difluoro-4-(6-{4-[(E)-(hydroxyimino)methyl]-1H-imidazol-1-yl}pyridin-3-yl)phenyl]-2-oxo-1,3-oxazolidin-5-yl}methyl)acetamide (Compound No. 69),

EIMS (m/z): 457.04 (M+H)
N-({(5S)-3-[2,6-difluoro-4′-(1,3-oxazol-5-yl)biphenyl-4-yl]-2-oxo-1,3-oxazolidin-5-yl}methyl)acetamide (Compound No. 70),

EIMS (m/z): 414.06 (M+H)
N-({(5S)-3-[2,6-difluoro-4′-(5-methyl-1,3,4-oxadiazol-2-yl)biphenyl-4-yl]-2-oxo-1,3-oxazolidin-5-yl}methyl)acetamide (Compound No. 71),

EIMS (m/z): 429.04 (M+H)
N-({(5S)-3-[2,6-difluoro-4′-(2-methyl-2H-tetrazol-5-yl)biphenyl-4-yl]-2-oxo-1,3-oxazolidin-5-yl}methyl)acetamide (Compound No. 72),

EIMS (m/z): 429.09 (M+H)
N-({(5S)-3-[2,6-difluoro-4′-(1-methyl-1H-tetrazol-5-yl)biphenyl-4-yl]-2-oxo-1,3-oxazolidin-5-yl}methyl)acetamide (Compound No. 73),

EIMS (m/z): 429.10 (M+H)
N-({(5S)-2-oxo-3-[2,3′,6-trifluoro-4′-(1-methyl-1H-tetrazol-5-yl)biphenyl-4-yl]-1,3-oxazolidin-5-yl}methyl)acetamide (Compound No. 74),

EIMS (m/z) 447.06 (M+H)
N-({(5S)-3-[2,3′-difluoro-4′-(1-methyl-1H-tetrazol-5-yl)biphenyl-4-yl]-2-oxo-1,3-oxazolidin-5-yl}methyl)acetamide (Compound No. 75),

EIMS (m/z): 429.10 (M+H)
N-({(5S)-2-oxo-3-[2,3′,6-trifluoro-4′-(2-methyl-2H-tetrazol-5-yl)biphenyl-4-yl]-1,3-oxazolidin-5-yl}methyl)acetamide (Compound No. 76),

EIMS (m/z): 447.14 (M+H)
N-({(5S)-3-[2,3′-difluoro-4′-(5-methyl-1,3,4-oxadiazol-2-yl)biphenyl-4-yl]-2-oxo-1,3-oxazolidin-5-yl}methyl)acetamide (Compound No. 77),

EIMS (m/z): 429.09 (M+H)
N-({(5S)-2-oxo-3-[2,3′,6-trifluoro-4′-(5-methyl-1,3,4-oxadiazol-2-yl)biphenyl-4-yl]-1,3-oxazolidin-5-yl}methyl)acetamide (Compound No. 78),

EIMS (m/z): 447.02 (M+H)
N-[((5S)-3-{3,5-difluoro-4-[2-(1H-1,2,3-triazol-1-yl)pyrimidin-5-yl]phenyl}-2-oxo-1,3-oxazolidin-5-yl)methyl]acetamide (Compound No. 79),

EIMS (m/z): 416.01 (M+H)
N-{[(5S)-3-(3,5-difluoro-4-{6-[5-(4-fluorophenyl)-1H-tetrazol-1-yl]pyridin-3-yl}phenyl)-2-oxo-1,3-oxazolidin-5-yl]methyl}acetamide (Compound No. 80),

EIMS (m/z): 510.04 (M+H)
Hydrochloride salt of N-({(5S)-3-[4′-(1H-benzimidazol-2-yl)-2,3′,6-trifluorobiphenyl-4-yl]-2-oxo-1,3-oxazolidin-5-yl}methyl)acetamide (Compound No. 81),

EIMS (m/z): 463.38 (M+H)
N-({(5S)-3-[2,3′-difluoro-4′-(2-methyl-2H-tetrazol-5-yl)biphenyl-4-yl]-2-oxo-1,3-oxazolidin-5-yl}methyl)acetamide (Compound No. 82),

EIMS (m/z): 429.38 (M+H)
N-[((5S)-3-{3,5-difluoro-4-[5-(2-methyl-2H-tetrazol-5-yl)-2-thienyl]phenyl}-2-oxo-1,3-oxazolidin-5-yl)methyl]acetamide (Compound No. 83),

EIMS (m/z): 435.36 (M+H)
N-{[(5S)-3-(3,5-difluoro-4-{6[-4-(hydroxymethyl)-1H-imidazol-1-yl]pyridin-3-yl}phenyl)-2-oxo-1,3-oxazolidin-5-yl]methyl}acetamide (Compound No. 84),

EIMS (m/z): 444.07 (M+H)
N-[((5S)-3-{3,5-difluoro-4-[2-(1H-pyrrol-1-yl)pyrimidin-5-yl]phenyl}-2-oxo-1,3-oxazolidin-5-yl)methyl]acetamide (Compound No. 85),

EIMS (m/z): 414.0 (M+H)
N-[((5S)-3-{3-fluoro-4-[2-(1H-pyrazol-1-yl)pyrimidin-5-yl]phenyl}-2-oxo-1,3-oxazolidin-5-yl)methyl]acetamide (Compound No. 86).

EIMS (m/z): 397.0 (M+H)

### Scheme VII

### Example 13

### Synthesis of tert-butyl [((5R)-3-{3,5-difluoro-4-[6-(1H-1,2,4-triazol-1-yl)pyridin-3-yl]phenyl}-2-oxo-1,3-oxazolidin-5-yl)methyl]isoxazol-3-ylcarbamate (Compound No. 87)

To [6-(1H-1,2,4-triazol-1-yl)pyridin-3-yl]boronic acid (79 mg) (obtained from Scheme III) and tert-butyl (4-{[(5R)-3-(3,5-difluoro-4-iodophenyl)-2-oxo-1,3-oxazolidin-5-yl]methyl}isoxazol-3-yl)carbamate (200 mg)(obtained from Scheme II) was added n-propanol (20 mL) and degassed with argon for 15 minutes. To the reaction mixture was added palladium diacetate (17 mg) and triphenyl phosphine (60 mg) and the reaction mixture was stirred under argon at room temperature for 15 min. To the reaction mixture was added sodium carbonate (40 mg) dissolved in degassed water and the reaction mixture was stirred at 100° C. for about 1.5 hours, cooled and filtered through celite. The filtrate was dissolved in ethyl acetate and the resulting organic layer was washed with aqueous sodium bicarbonate solution and brine, and dried over anhydrous sodium sulfate. The solvent was concentrated under vacuum and the residue was triturated over diethyl ether to yield the title compound (50 mg).

¹HNMR (CDCl3): δ 9.22 (s, 1H), 8.56 (s, 1H), 8.27 (s, 1H), 8.12 (s, 1H), 8.00 (s, 2H), 7.32 (m, 2H), 6.9 (bs, 1H), 5.13 (m, 1H), 4.39 (m, 1H), 4.16 (m, 2H), 3.87 (m, 1H), 1.56 (s, 9H);

EIMS (m/z) 540.37 (M+H)

Analogue of tert-butyl [((5R)-3-{3,5-difluoro-4-[6-(1H-1,2,4-triazol-1-yl)pyridin-3-yl]phenyl}-2-oxo-1,3-oxazolidin-5-yl)methyl]isoxazol-3-ylcarbamate described below was prepared by replacing [6-(1H-1,2,4-triazol-1-yl)pyridin-3-yl]boronic acid with appropriate boronic acids as applicable in each case.
tert-butyl [((5R)-3-{3-fluoro-4-[6-(1,3-oxazol-5-yl)pyridin-3-yl]phenyl}-2-oxo-1,3-oxazolidin-5-yl)methyl]isoxazol-3-ylcarbamate (Compound No. 88)

EIMS (m/z): 539.25 (M+H)

### Example 14

### Synthesis of (5S)-3-{3,5-difluoro-4-[6-(2-methyl-2H-tetrazol-5-yl)pyridin-3-yl]phenyl}-5-[(isoxazol-3-ylamino)methyl]-1,3-oxazolidin-2-one (Compound No. 89)

### Step a: Synthesis of tert-butyl [((5R)-3-{3,5-difluoro-4-[6-(2-n2-ethyl-2H-tetrazol-5-yl)pyridin-3-yl]phenyl}-2-oxo-1,3-oxazolidin-5-yl)methyl]isoxazol-3-ylcarbamate

To a mixture of [6-(2-methyl-1H-triazol-1-yl)pyridin-3-yl]boronic acid (89 mg) (obtained from scheme III) and tert-butyl {[(5R)-3-(3,5-difluoro-4-iodophenyl)-2-oxo-1,3-oxazolidin-5-yl]methyl}isoxazol-3-ylcarbamate (210 mg)(obtained from scheme II) under argon was added n-propanol (25 mL). The reaction mixture was degassed with argon for about 15 minutes. To the reaction mixture was added palladium diacetate (17 mg) and triphenyl phosphine (60 mg) and the reaction mixture was stirred under argon at room temperature for an additional 15 minutes. Sodium carbonate (40 mg) dissolved in degassed water was then added to the reaction mixture and stirred at 100° C. for about 1.5 hours, cooled and filtered. The reaction mixture was quenched with ethyl acetate and the organic layer was washed with aqueous sodium bicarbonate and brine. The organic extract was dried over anhydrous sodium sulfate. The solvent was concentrated and the slurry was triturated over diethyl ether to yield title compound as white solid. (95 mg).

### Step b: Synthesis of (5S)-{3-3,5-difluoro-4-[6-(2-methyl-2H-tetrazol-5-yl)pyridin-3-yl]phenyl}-5-[(isoxazol-3-ylamino)methyl]-1,3-oxazolidin-2-one

The compound obtained from step a above (90 mg) was dissolved in ethanolic HCl (30 mL) and the reaction mixture was stirred at room temperature for 3 hours. The solvent was concentrated and the residue was triturated over ether to yield the title compound. (50 mg).

¹HNMR (CDCl3): δ 8.92 (s, 1H), 8.42 (m, 2H), 8.23 (m, 1H), 7.57 (m, 2H), 6.02 (m, 1H), 4.96 (m, 1H), 4.47 (s, 3H), 4.23 (m, 1H), 3.89 (m, 1H), 3.47 (m, 2H).

EIMS (m/z) 455.17 (M+H)

Analogues of (5S)-3-{3,5-difluoro-4-[6-(2-methyl-2H-tetrazol-5-yl)pyridin-3-yl]phenyl}-5-[(isoxazol-3-ylamino)methyl]-1,3-oxazolidin-2-one described below were prepared by replacing [6-(2-methyl-1H-triazol-1-yl)pyridin-3-yl]boronic acid or tert-butyl {[(5R)-3-(3,5-difluoro-4-iodophenyl)-2-oxo-1,3-oxazolidin-5-yl]methyl}isoxazol-3-ylcarbamate with appropriate boronic acids and carbamate as applicable in each case:
(5S)-3-[2,3′-difluoro-4′-(1,3-oxazol-5-yl)biphenyl-4-yl]-5-[(isoxazol-3-ylamino)methyl]-1,3-oxazolidin-2-one (Compound No. 90),

EIMS (m/z): 439.27 (M+H);
(5S)-3-{3,5-difluoro-4-[6-(1H-imidazol-1-yl)pyridin-3-yl]phenyl}-5-[(isoxazol-3-ylamino)methyl]-1,3-oxazolidin-2-one (Compound No. 91),

EIMS (m/z) 439.18 (M+H);
(5S)-5-[(isoxazol-3-ylamino)methyl]-3-[2,3′,6-trifluoro-4′-(4-phenyl-1H-imidazol-1-yl)biphenyl-4-yl]-1,3-oxazolidin-2-one (Compound No. 92),

EIMS (m/z) 532.17 (M+H);
(5S)-3-{3,5-difluoro-4-[6-(1-methyl-1H-tetrazol-5-yl)pyridin-3-yl]phenyl}-5-[(isoxazol-3-ylamino)methyl]-1,3-oxazolidin-2-one (Compound No. 93).

EIMS (m/z) 455.23 (M+H).

### Scheme VIII

### Example 15

### Synthesis of N-[((5S)-3-{3,5-difluoro-4-[6-(1H-1,2,4-triazol-1-yl)pyridin-3-yl]phenyl}-2-oxo-1,3-oxazolidin-5-yl)methyl]ethanethioamide (Compound No. 94),

Acetamide derivative (100 mg) obtained from Scheme IV Example 4 was dissolved in dioxane and heated to 95° C. Lawesson's reagent (121 mg) was added to the heated reaction mixture and then stirred at 95° C. for about 2 hours. The solvent was removed under vacuum and the reaction mixture was dissolved in dichloromethane and washed with aqueous sodium bicarbonate solution followed by brine. The solvent was removed and the crude product was purified by column chromatography using 0.2% methanol/dichloromethane as eluant to yield the title compound (35 mg).

¹HNMR (DMSO d6): δ 10.38 (s, 1H), 9.43 (s, 1H), 8.65 (s, 1H), 8.34 (s, 1H), 8.22 (m, 1H), 8.03 (m, 1H), 7.54 (d, 2H, 12 Hz), 5.03 (m, 1H), 4.27 (m, 1H), 3.95 (m, 3H), 2.50 (s, 3H);

EIMS (m/z) 431.36 (M+H)

Analogue of N-[((5S)-3-{3,5-difluoro-4-[6-(1H-1,2,4-triazol-1-yl)pyridin-3-yl]phenyl}-2-oxo-1,3-oxazolidin-5-yl)methyl]ethanethioamido described below was prepared by replacing with appropriate methylthioacetamide as applicable in each case.
N-({(5S)-3-[2,3′-difluoro-4′-(1,3-oxazol-5-yl)biphenyl-4-yl]-2-oxo-1,3-oxazolidin-5-yl}methyl)ethanethioamide (Compound No. 95),

EIMS (m/z) 430.34 (M+H).

### Example 16

### Synthesis of (5S)-5-(aminomethyl)-3-{3,5-difluoro-4-[6-(1H-1,2,4-triazol-1-yl)pyridin-3-yl]phenyl}-1,3-oxazolidin-2-one (Compound No. 96)

(S)-[N-3-(4-(2-(1,2,4-Triazol-1-yl)pyridine-5-yl)-3,5-difluorophenyl)-2-Oxo-5-oxazolidinyl]methylacetamide (800 mg) was suspended in absolute ethanol (20 mL) and refluxed at 80° C. with 20% aqueous HCl (4 mL) for 12 hours. The reaction mixture was cooled and basified with ammonia to obtain a white precipitate. The precipitate was extracted with ethyl acetate and the solvent was evaporated under vacuum to yield the title compound. (400 mg).

¹HNMR (DMSO d6): δ 9.42 (s, 1H), 8.64 (s, 1H), 8.33 (s, 1H), 8.21 (m, 1H), 8.03 (m, 1H), 7.53 (d, 2H, 10.2 Hz), 4.69 (m, 1H), 4.13 (m, 1H), 3.93 (m, 1H), 3.53 (m, 2H), 2.88 (bs, 2H);

EIMS (m/z): 373.41 (M+H)

### Example 17

### Synthesis of methyl [((5S)-3-{3,5-difluoro-4-[6-(1H-1,2,4-triazol-1-yl)pyridin-3-yl]phenyl}-2-oxo-1,3-oxazolidin-5-yl)methyl]carbamate (Compound No. 97)

The compound obtained from the Example 14 (100 mg) was dissolved in dichloromethane and the solution was cooled to 0° C. Methyl chloroformate (0.2 mL) was added and the reaction mixture was stirred at 25° C. for about 3 hours. The reaction mixture was washed with water, the solvent was evaporated under vacuum and the crude product was purified by column chromatography using dichloromethane/methanol as eluant to yield the title compound (70 mg).

¹HNMR (DMSO d6): δ 9.42 (s, 1H), 8.65 (s, 1H), 8.34 (s, 1H), 8.21 (m, 1H), 8.03 (m, 1H), 7.52 (m, 3H), 4.80 (m, 1H), 4.22 (m, 1H), 4.17 (m, 1H), 3.83 (m, 5H);

EIMS (m/z) 431.50 (M+H)

Analogue of methyl [((5S)-3-{3,5-difluoro-4-[6-(1H-1,2,4-triazol-1-yl)pyridin-3-yl]phenyl}-2-oxo-1,3-oxazolidin-5-yl)methyl]carbamate described below was prepared by replacing appropriate methyl carbamic acid methylester as applicable in each case
methyl ({(5S)-3-[2,3′-difluoro-4′-(1,3-oxazol-5-yl)biphenyl-4-yl]-2-oxo-1,3-oxazolidin-5-yl}methyl)carbamate (Compound No. 98),

EIMS (m/z) 430.38 (M+H)

### Example 18

### Synthesis of (5S)-3-{3,5-difluoro-4-[6-(1,3-oxazol-5-yl)pyridin-3-yl]phenyl}-5-(isothiocyanatomethyl)-1,3-oxazolidin-2-one (Compound No. 99),

To a solution of (S)N-3-(4-(2-(1,2,4-triazol-1-yl)pyridine-5-yl)-3,5-difluorophenyl)-2-oxo-5-oxazolidinyl]methylamine (500 mg) in tetrahydrofuran (50 mL) was added triethylamine (0.27 mL) followed by carbon disulfide (0.16 mL) at 10° C. The reaction mixture was stirred at 25° C. for 12 hours. The reaction mixture was quenched with ethyl chloroformate (0.128 mL) and water, and then extracted with ethyl acetate, washed with brine, and dried over sodium sulfate. The solvent was removed and the crude product was purified by column chromatography using 0.2% dichloromethane/methanol as eluant to yield the title compound (480 mg).

¹HNMR (DMSO d₆): δ 9.43 (s, 1H), 8.66 (s, 1H), 8.34 (s, 1H), 8.23 (d, 1H, 8.4 Hz), 8.03 (d, 1H, 8.4 Hz), 7.56 (d, 2H, 10.2 Hz), 5.04 (m, 1H), 4.26 (m, 1H), 4.07 (m, 1H), 3.90 (m, 2H);

EIMS (m/z) 415 (M+H)

### Example 19

### Synthesis of (N-[((5S)-3-{3,5-difluoro-4-[6-(1H-1,2,4-triazol-1-yl)pyridin-3-yl]phenyl}-2-oxo-1,3-oxazolidin-5-yl)methyl]thiourea (Compound No. 100)

To a solution of (S)-[N-3-(4-(2-(1,2,4-triazol-1-yl)pyridine-5-yl)-3,5-difluorophenyl)-2-oxo-5-oxazolidinyl]methylisothiocyanate (150 mg) in methanol (100 mL) was added methanolic ammonia (10 mL) at 0° C. The reaction mixture was stirred at 0° C. for about 4 hours. The solid that separated out was filtered and dried under vacuum to yield the title compound. (75 mg)

¹HNMR (DMSO d₆): δ 9.43 (s, 1H), 8.66 (s, 1H), 8.34 (s, 1H), 8.22 (m, 1H), 8.00 (m, 2H), 7.54 (d, 211, 9 Hz), 4.48 (m, 1H), 4.21 (m, 1H), 4.10 (m, 1H), 3.90 (m, 2H);

EIMS (m/z) 432.33 (M+H)

### Example 20

### Synthesis of N-[((5S)-3-{3,5-difluoro-4-[6-(1H-1,2,4-triazol-1-yl)pyridin-3-yl]phenyl}-2-oxo-1,3-oxazolidin-5-yl)methyl]-N′-methylthiourea (Compound No. 101)

To a solution of (S)N-3-(4-(2-(1,2,4-triazol-1-yl)pyridine-5-yl)-3,5-difluorophenyl)-2-Oxo-5-oxazolidinyl]methylisothiocyanate (100 mg) in methanol was added triethylamine (0.56 mL) followed by methyl amine hydrochloride (200 mg) at 25° C. The reaction mixture was stirred for 3 hours at 25° C. and the solvent was evaporated under vacuum. The residue thus obtained was dissolved in dichloromethane, washed with water, dried over sodium sulfate. The solvent was removed under vacuum to yield the title compound (78 mg).

¹HNMR (DMSO d6): δ 9.43 (s, 1H), 8.65 (s, 1H), 8.34 (s, 1H), 8.22 (d, 111, 9 Hz), 8.03 (d, 111, 9 Hz), 7.78 (m, 1H), 7.54 (d, 2H, 9 Hz), 4.94 (m, 1H), 4.23 (m, 1H), 4.17 (m, 1H), 3.90 (m, 2H), 3.42 (s, 2H);

EIMS (m/z) 446.37 (M+H)

### Example 21

### Assay for In Vitro Antibacterial Activity

The compounds of the invention display antibacterial activity when tested by the agar incorporation method. The following minimum inhibitory concentrations (μg/mL) were obtained for representative compounds of the invention:

S. aureus ATCC 25923—Staphylococcus aureus ATCC 25923
S. aureus ATCC 15187—Staphylococcus aureus ATCC 15187
MRSA—Methicilline Resistant Staphylococcus aureus ATCC562
MRSA—Methicilline Resistant Staphylococcus aureus ATCC33
Ent. faecalis ATCC 29212-Enterococcus faecalis ATCC 29212
Pseudomonas aeruginosa ATCC 27853
Streptococcus pneumoniae ATCC 49619
Strep. pyog. ATCC 19615—Streptococcus pyogenes
S. aureus ATCC 25923—Staphylococcus aureus ATCC 25923
Ent. faecium 6A—Enterococcus faecium 6A Van®, Cipro®
Strep. pneum. ATCC 6303—Streptococcus pneumoniae ATCC 6303
Strep. pyog. ATCC 19615-Streptococcus pyogenes
B. fragillus—Bacillus fragillus ATCC 25285
M. catt.—Moraxella catarrhalis ATCC 8176
VRE—Vancomycin-resistant enterococci ATCC 6A
H. influ.—Haemophilus influenzae

TABLE
In vitro (μg/mL)

| MIC | S. aureus | S. aureus | MRSA | MRSA | E. faecalis | VRE | S. pyogenes | S. pneum |
|---|---|---|---|---|---|---|---|---|
| Range | 25923 | 15187 | 562 | 33 | 29212 | 6A | 19615 | 6303 |
| 1 | 0.25-16 | 0.25-16 | 0.25-16 | 0.25-16 | 0.25-16 | 0.13-16 | 0.03-16 | 0.06-16 |
| 2 | 0.25-2 | 0.25-2 | 0.25-2 | 0.25-2 | 0.25-2 | 0.13-4 | 0.03-2 | 0.06-4 |
| 3 | 0.25-1 | 0.25-1 | 0.25-1 | 0.25-1 | 0.25-1 | 0.5-2 | 0.03-1 | 0.06-1 |

| | | | | S. aureus | | S. pneum | |
|---|---|---|---|---|---|---|---|
| MIC | S. pneum | M. catt | H. inf | MRSA | E. faecium | 6303 | B. fragilis |
| Range | AB34 | ATCC8176 | 49247 | 32LNZr | 303 LNZr | LNZr | 25285 |
| 1 | 0.06-16 | 0.06-32 | 2-32 | 0.25-16 | 0.25-16 | 0.25-32 | 2-16 |
| 2 | 0.06-4 | 0.06-8 | 2-16 | 0.25-8 | 0.25-4 | 0.25-4 | 2-8 |
| 3 | 0.06-1 | 0.06-4 | 2-8 | 0.25-4 | 0.25-2 | 0.25-2 | 2-4 |

The in vitro antibacterial activity of the compounds was demonstrated by the agar dilution method (NCCLS M 7 and M 100-S8 documents). Briefly, the compounds were dissolved in dimethylsulfoxide and doubling dilution of the compounds were incorporated into Muller Hilton agar before solidification. Inoculum was prepared by direct colony suspension in normal saline solution and adjusting the turbidity to 0.5 Macfarland turbidity and subsequently diluting as per NCCLS guidelines in order to obtain 10⁴ CFU/spot. CFU/mL of few randomly selected cultures was performed. The cultures were replicated Denley's multipoint replicator. The agar plates were incubated for 18 hours-24 hours (24 hours for MRSA studies) at 35±2° C. Q.C. strains were also included in each run of the study.

The in vitro activity for Haemophilus MICs were performed by using Micro broth dilution method as follows:

Media used: Mueller Hinton Broth (MHB-Difco)−Cation adjusted+5 grams per liter Yeast extract+supplements
Preparation of drug concentrations in 96 well microtitre plates was done as per the NCCLS method. Inoculum was prepared by direct colony suspensions in normal saline and adjusted to 1 McFarland turbidity and subsequently diluted in broth 100 times as per NCCLS guidelines in order to obtain 105 CFU/spot. The concentration showing no growth of the inoculated culture was recorded as the MIC. Appropriate ATCC standard strains were simultaneously tested and result recorded only when the MIC's against standard antibiotics were within the acceptable range. While the present invention has been described in terms of its specific embodiments, certain modifications and equivalents will be apparent to those skilled in the art and are intended to be included within the scope of the present invention.

1. A compound having the structure of Formula I, 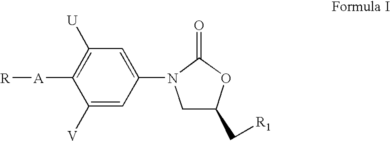
or a pharmaceutically acceptable salt, pharmaceutically acceptable solvate, ester, enantiomer, diastereomer, poly morph, prodrug or metabolite thereof, wherein
A is 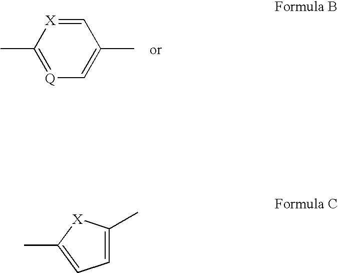
wherein Q and X are independently selected from —N—, —O—, —C—F, —CH— or —S—;
U and V are independently selected from hydrogen (wherein both U and V cannot be H at the same time), lower (C₁₋₆) alkyl or halogen;
R is CH═NOR_{f}, CH═NOC(═O)R_{f}, CH═NOSO₂R_{f}, CH═NOC(═O)NHR_{f}, heterocyclyl or heteroaryl, wherein
R_{f} is hydrogen, alkyl, alkenyl, alkynyl, alkoxy, cycloalkyl, aryl, aralkyl, heteroaryl, heterocyclyl, heteroarylalkyl or heterocyclylalkyl;
R₁ is azide, NCS, NHYR_{f}, NRⱼ C(=T)NR_{f}R_{q}, NR_{f}R_{q}, or NRⱼ(C═O)ORₛ; wherein
Y is (C═O), (C═S) or SO₂,
R_{f} is the same as defined earlier,
T is O, S, —N(CN), —N(NO₂), or —CH(NO₂),
Rⱼ is hydrogen, alkyl, alkenyl, cycloalkyl, aryl, aralkyl, heterocyclyl, heteroaryl, heteroarylalkyl or heterocyclylalkyl,
R_{q} is hydrogen, alkyl, cycloalkyl, aryl, aralkyl, heteroaryl, heterocyclyl, heteroarylalkyl or heterocyclyl alkyl, and
Rₛ is alkyl, alkenyl, alkynyl, cycloalkyl, aryl, aralkyl, heteroarylalkyl or heterocyclylalkyl,
with the proviso that:
when U is H, V is F, R₁ is NHCOCH₃ and A is Formula B (wherein Q or X is N), then R is a five membered heteroaryl ring containing two or four N atoms (wherein the five membered heteroaryl ring containing four N atom is linked through N-atom to Formula B and is always substituted);
when A is Formula B (wherein Q and X both are N) and U,V and R₁ are as defined above then R cannot be a live membered heterocyclyl ring containing 2 hetero atoms.

2. The compound according to claim 1, wherein R₁ is selected from amino, isothiocyanate, tert-butyl isoxazol-3-yl carbamate, isoxzol-3-amine, ethanethioamido, acetamide, thiourea, N-methylthiourea or methyl carbamate.

3. The compound according to claim 1, wherein V and U are independently selected from hydrogen or fluorine.

4. The compound of claim 1, wherein A is substituted heteroaryl.

5. The compound of claim 1, wherein A is selected from pyridinyl, monofluorophenyl, pyrimidinyh furanyl or thiophenyl.

6. The compound of claim 1, wherein R is optionally substituted heteroaryl.

7. The compound of claim 1, wherein R is selected from 2-methyl-2H-tetrazolyl, 1-methyl-1H-tetrazolyl, 1H-1,2,4-triazolyl, 1,3-oxazolyl, 1H-imidazolyl, 5-phenyl-1H-tetrazoyl, 3a,7a-dihydro-1H-benzimidazolyl, 3-(1H-imidazol-4-yl)pyridine, oxazol-5-yl methanol, 5-methyl-5-tetrazole, (5R)-5-(hydroxymethyl)-1,3-oxazolidin-2-one, 1-methyl-2-phenyl-1H-imidazole, 2-methyl-1,3,4-oxadiazole, N-1,3,4-thaidiazole-2-yl acetamide, 1H-pyrrol-3-yl methanol, 1H-pyrrole-3-carbaldehyde, 1H-pyrrole-3-carbaldehyde oxime, (1E)-acetaldehyde O-(3,4-difluororbenzyl)oxime, (1E) acetaldehyde O-acetyloxime, (1E)-acetaldehyde O-benzoyl oxime, (1Z)-acetaldehyde-O ({[4-(trifluoromethyl)-phenyl]-amino}carbonyl)oxime, (1E)-acetaldehyde O-[(tert-butyl amino)-carbonyl]-oxime or (1Z)-acetaldehyde-O-{[(4-fluorophenyl)amino]carbonyl}-oxime.

8. A compound selected from
N-[((5S)-3-{3,5-difluoro-4-[6-(3-formyl-1H-pyrrol-1-yl)pyridin-3-yl]phenyl}-2-oxo-4,3-oxazolidin-5-yl)methyl]acetamide (Compound No. 1),
N-{[(5S)-3-(3,5-difluoro-4-{6-[3-(hydroxymethyl)-1H-pyrrol-1-yl]pyridin-3-yl}phenyl)-2-oxo-1,3-oxazolidin-5-yl)methyl}acetamide (Compound No. 2),
N-({(5S)-3-[3-fluoro-4-(2-{3-[(E)(hydroxyimino)methyl]-1H-pyrrol-1-yl}pyrimidin-5-yl)phenyl]-2-oxo-1,3-oxazolidin-5-yl}methyl)acetamide (Compound No. 3),
N-({(5S)-3-[3,5-difluoro-4-(2-{3-(E)-(hydroxyimino)methyl]-1H-pyrrol-1-yl}pyrimidin-5-yl)phenyl]-2-oxo-1,3-oxazolidin-5-yl}methyl)acetamide (Compound No. 4),
N-({(5S)-3-[4′-((E)-{[(3,4-difluorobenzyl)oxy]imino}methyl)-2,3′,6-trifluorobiphenyl-4-yl]-2-oxo-1,3-oxazolidin-5-yl}methyl)acetamide (Compound No. 5),
N-{[(5S)-3-(4′-{(E)-[acetyloxy)imino]methyl}-2,3′,6-trifluorobiphenyl-4-yl)-2-oxo-1,3-oxazolidin-5-yl]methyl}acetamide (Compound No. 6),
N-{[(5S)-3-(4′-{(E)-[benzoyloxy)imino]methyl}-2,3′,6-trifluorobiphenyl-4-yl)-2-oxo-1,3-oxazolidin-5-yl]methyl}acetamide (Compound No. 7),
N-({(5S)-2-oxo-3-[2,3′,6-trifluoro-4-((E)-{[(methylsulfonyl)oxy]imino}methyl)biphenyl-4-yl]-1,3-oxazolidin-5-yl}methyl)acetamide (Compound No. 8),
N-({(5S)-2-oxo-3-[2,3′,6-trifluoro-4′-((E)-{[({[4-(trifluoromethyl)phenyl]amino}carbonyl)oxy]imino}methyl)biphenyl-4-yl]-1,3-oxazolidin-5-yl}methyl)acetamide (Compound No. 9),
N-[((5S)-3-{4′-[(E)-({[(tert-butylamino)carbonyl]oxy}imino)methyl]-2,3′,6-trifluorobiphenyl-4-yl}-2-oxo-1,3-oxazolidin-5-yl)methyl]acetamide (Compound No. 10),
N-{[(5S)-2-oxo-3-(2,3′,6-trifluoro-4′-{(E)-[({[(4-fluorophenyl)amino]carbonyl}oxy)imino]methyl}biphenyl-4-yl)-1,3-oxazolidin-5-yl]methyl}acetamide (Compound No. 11),
N-({(5S)-2-oxo-3-[2,3′,6-trifluoro-4′-(1,3-oxazol-5-yl)biphenyl-4-yl]-1, oxazolidin-5-yl}methyl)acetamide (Compound No. 12),
N-[((5S)-3-{3,5-difluoro-4-[6-(1H-1,2,4-triazol-1-yl)pyridin-3-yl]phenyl}2-oxo-1,3-oxazolidin-5-yl)methyl]acetamide (Compound No 13),
N-{[(5S)-3-(3,5-difluoro-4-[6-[5-(1,3-oxazol-4-yl)-2-furyl]pyridin-3-yl]phenyl)-2-oxo-1,3-oxazolidin-5-yl]methyl}acetamide (Compound No 14),
N-{[(5)-3-(3,5-difluoro-4-{6-[5-(1,3-oxazol-4-yl)-2-thienyl]pyridin-3-yl}phenyl)-2-oxo-1,3-oxazolidin-5-yl]methyl}acetamide (Compound No. 15),
N-{[(5S)-3-(3,5-difluoro-4-{6-[3-(hydroxymethyl)-1H-pyrrol-1-yl]pyridin-3-yl}phenyl)-2-oxo-1,3-oxazolidin-5-yl]methyl}acetamide (Compound No. 16),
N-({(5S)-3-[3,5-difluoro-4-(6-{3-[(E)-(hydroxyimino)methyl]-1H-pyrrol-1-yl}pyridin-3-yl]phenyl]-2-oxo-1,3-oxazolidin-5-yl}methyl)acetamide (Compound No. 17),
N-({(5S)-3-[2,3′-difluoro-4′-(5-methyl-1,2,4-oxadiazol-3-yl)phenyl-4-yl]-2-oxo-1,3-oxazolidin-5-yl}-methyl)acetamide (Compound No. 18),
N-({(5S)-2-oxo-3-[2,3′,6-trifluoro-4′-(5-methyl-1,2,4-oxadiazol-3-yl)biphenyl-4-yl]-1,3-oxazolidin-5-yl}methyl)acetamide (Compound No. 19),
N-[((5S)-3-{3,5-difluoro-4-(6-(2-methyl-2H-tetrazol-5-yl)pyridin-3-yl]phenyl}-2-oxo-1,3-oxazolidin-5-yl)methyl]acetamide (Compound No. 20),
N-[((5S)-3-{3,5-difluoro-4-[6-(1H-imidazol-1-yl)pyridin-3-yl]phenyl]-2-oxo-1,3-oxazolidin-5-yl)methyl}acetamide (Compound No. 21),
N-[((5S)-3-{3,5-difluoro-4-[6-(1-methyl-1H-tetrazol-5-yl)pyridin-3-yl]phenyl}-2-oxo-1,3-oxazolidin-5-yl)methyl]acetamide (Compound No. 22),
(5S)-3-{3,5-difluoro-4-[6-(2-methyl-2H-tetrazol-5-yl)pyridin-3-yl]phenyl}-5-[(1,3-thiazol-2-ylamino)methyl]-1,3-oxazolidin-2-one (Compound No. 23),
N-{[(5S)-3-(2,3′-difluoro-4′-{3-[(E)-(hydroxyimino)methyl]-1H-pyrrol-1-yl}biphenyl-4-yl)-2-oxo-1,3-oxazolidin-5-yl)methyl}acetamido (Compound No. 24),
N-({(5S)-3-[2,3′-difluoro-4′-(5-methyl-1H-tetrazol-1-yl)biphenyl-4-yl]-2-oxo-1,3-oxazolidin-5-yl}methyl)acetamide (Compound No. 25),
N-((S)-3-{3,5-Difluoro-4-[6-[5-methyl-[1,3,4]oxadiazol-2-yl)-pyridin-3-yl]-phenyl}-2-oxo-oxazolidin-5-yl-methyl)-acetamide (Compound No. 26),
N-((S)-3-{4-[6-(5-Amino-[1,3,4-thiadiazol-2-yl)-pyridin-3-yl]-3,5-difluoro-phenyl}-2-oxo-oxazolidin-5-ylmethyl)-acetamide (Compound No. 27),
N-({(5S)-3-[4′-(1H-benzimidazol-2-yl)-2,3′,6-trifluorobiphenyl-4-yl]-2-oxo-1,3-oxazolidin-5-yl}methyl)acetamido (Compound No. 28),
N-[((5S)-3-{3,5-difluoro-4-[6-(1H-1,2,3-triazol-1-yl)pyridin-3-yl]phenyl}-2-oxo 1,3-oxazolidin-5-yl)methyl]acetamide (Compound No. 29),
N-[((5S)-3-(3,5-difluoro-4-[6-(4-phenyl-1H-imidazol-1-yl)pyridin-3-yl]phenyl)-2-oxo-1,3-oxazolidin-5-yl)methyl]acetamide (Compound No. 30),
N-[((5S)-3-{3-fluoro-4-[5-(2-methyl-2H-tetrazol-5-yl)-2-furyl]phenyl}-2-oxo-1,3-oxazolidin-5-yl)methyl]acetamide (Compound No. 31),
N-[((5S)-3-{3-fluoro-4-[5-(1-methyl-1H-tetrazol-5-yl)-2-furyl]phenyl}-2-oxo-1,3-oxazolidin-5-yl)methyl]acetamide (Compound No. 32),
N-[((5S)-3-{3,5-difluoro-4-[5-(3-methyl-2,3-dihydro-1H-tetrazol-5-yl)-2-furyl]phenyl}-2-oxo-1,3-oxazolidin-5-yl)methyl]acetamide (Compound No. 33),
N-({(5S)-2-oxo-3-[2,3′,6-trifluoro-4′-(1-methyl-1H-benzimidazol-2-yl)biphenyl-4-yl]-1,3-oxazolidin-5-yl}methyl)acetamide (Compound No. 34),
N-[5-(4′-{(5S)-5-[(acetylamino)methyl]-2-oxo-1,3-oxazolidin-3-yl}-2′,3,6′-trifluorobiphenyl-4-yl)-1,3,4-thiadiazol-2-yl]acetamide (Compound No. 35),
N-({(5S)-3-[2,3′-difluoro-4′-(1,3-thiazol-2-yl)biphenyl-4-yl]-oxo-1,3-oxazolidin-5-yl}methyl)acetamide (Compound No. 36),
N-[(3-{2,3′-difluoro-4′-[5-(hydroxymethyl)-4,5-dihydroisoxazol-3-yl]biphenyl-4-yl}-2-oxo-1,3-oxazolidin-5-yl)methyl]acetamide (Compound No. 37),
N-[((5S)-3-{3-fluoro-4-[2-(1H-imidazol-1-yl)pyrimidin-5-yl]phenyl}-2-oxo-1,3-oxazolidin-5-yl)methyl]acetamide (Compound No. 38),
N-({(5S)-2-oxo-3-[2,3′,6-trifluoro-4*-(1,3-thiazol-2-yl)biphenyl-4-yl]-1,3-oxazolidin-5-yl}methyl)acetamide (Compound No. 39),
N-[((5S)-2-oxo-3-{2,3′,6-trifluoro-4′-[(5S)-5-(hydroxymethyl)-2-oxo-1,3-oxazolidin-3-yl]biphenyl-4-yl}-1,3-oxazolidin-5-yl)methyl]acetamide (compound No. 40),
N-({(5S)-3-[2,3′-difluoro-4′-(5-phenyl-1H-tetrazol-1-yl)biphenyl-4-yl]-2-oxo-1,3-oxazolidin-5-yl}methyl)acetamide (Compound No. 41),
N-[(3-{3-fluoro-4-[6-(5-phenyl-1H-tetrazol-1-yl)pyridin-3-yl]phenyl}-2-oxo-1,3-oxazolidin-5-yl)methyl]acetamiude (Compound No. 42),
N-[((5S)-2-oxo-3-{2,3′,6-trifluoro-4′-[5-(hydroxymethyl)isoxazol-3-yl]biphenyl-4-yl}-1,3-oxazolidin-5-yl)methyl]acetamide (Compound No. 43),
N-[((5S)-3-{2,3′-difluoro-4′-[5-(hydroxymethyl)isoxazol-3-yl]biphenyl-4-yl}-2-oxo-1,3-oxazolidin-5-yl)methyl]acetamide (Compound No. 44),
N-[((5S)-3-{3,5-difluoro-4-[6-(4-pyridin-3-yl-1H-imidazol-1-yl]pyridin-3-yl]phenyl}-2-oxo-1,3-oxazolidin-5-yl)methyl]acetamide (Compound No. 45),
N-[((5S)-3-{3,5-difluoro-4-[6-(5-phenyl-1H-tetrazol-1-yl)pyridin-3-yl]phenyl}-2-oxo-1,3-oxazolidin-5-yl)methyl]acetamide (Compound No. 46),
N-[((5S)-3-{4-[2-(1H-benzimidazol-1-yl)pyrimidin-5-yl]-3,5-difluorophenyl}oxo-1,3-oxazolidin-5-yl)methyl]acetamide (Compound No. 47),
N-[((5S)-3-{4-[2-(1H-benzimidazol-1-yl)pyrimidin-5-yl]-3-fluorophenyl}-2-oxo-1,3-oxazolidin-5-yl)methyl]acetamide (Compound No. 48),
N-[((5S)-3-{3,5-difluoro-4-[2-(1H-1,2,4-triazol-1-yl)pyrimidin-5-yl]phenyl}-2-oxo-1,3-oxazolidin-5-yl)methyl]acetamide (Compound No. 49),
N-[((5S)-3-{3-fluoro-4-[2-(4-phenyl-1H-imidazol-1-yl)pyrimidin-5-yl]phenyl}-2-oxo-1,3-oxazolidin-5-yl)methyl]acetamide (Compound No. 50),
N-[((5S)-3-{3-fluoro-4-[2-(1H-1,2,4-triazol-1-yl)pyrimidin-5-yl]phenyl}-2-oxo-1,3-oxazolidin-5-yl)methyl]acetamide (Compound No. 51),
N-({(5S)-2-oxo-3-[2,3′,6-trifluoro-4′-(5-phenyl-1H-tetrazol-1-yl)biphenyl-4-yl]-1,3-oxazolidin-5-yl}methyl)acetamide (Compound No. 52),
N-[((5S)-3-{3,5-difluoro-4-[2-(4-phenyl-[1H-imidazol-1-yl)pyrimidin-5-yl]phenyl}-2-oxo-1,3-oxazolidin-5-yl)methyl]acetamide (Compound No. 53),
N-[((5S)-3-{3,5-difluoro-4-[2-(2-oxo-1,3-oxazolidin-3-yl)pyrimidin-5-yl]phenyl}-2-oxo-1,3-oxazolidin-5-yl)methyl]acetamide (Compound No. 54),
N-[((5S)-3-{3-fluoro-4-[2-(1H-1,2,3-triazol-1-yl)pyrimidin-5-yl]phenyl}-2-oxo-1,3-oxazolidin-5-yl)methyl]acetamide (Compound No. 55),
N-[((5S)-3-{4-[2-(3,5-dimethyl-1H-pyrazol-1-yl)pyrimidin-5-yl]-3-fluorophenyl}-1,3-oxazolidin-5-yl)methyl]acetamide (Compound No. 56),
N-[((5S)-3-{3-fluoro-4-[2-(2-oxo-1,3-oxazolidin-3-yl)pyrimidin-5-yl]phenyl}-2-oxo-1,3-oxazolidin-5-yl)methyl]acetamide (Compound No. 57),
N-{[(5S)-3-(4-{6-[4-(difluoromethyl)-1H-imidazol-1-yl]pyridin-3-yl}-3,5-difluorophenyl)-2-oxo-1,3-oxazolidin-5-yl]methyl}acetamide (Compound No. 58),
N-[((5S)-3-{3-fluoro-4-[2-(3-formyl-1H-pyrrol-1-yl)pyrimidin-5-yl]phenyl}-2-oxo-1,3-oxazolidin-5-yl)methyl]acetamide (Compound No. 59),
N-({(5S)-2-oxo-3-[2,3′,6-trifluoro-4′-(1H-1,2,4-triazol-1-yl)biphenyl-4-yl]-1,3-oxazolidin-5-yl}methyl)acetamide (Compound No. 60),
N-[((5S)-3-{3,5-difluoro-4-[2-(3-formyl-1H-pyrrol-1-yl)pyrimidin-5-yl]phenyl}-2-oxo-1,3-oxazolidin-5-yl)methyl]acetamide (Compound No. 61),
N-[((5S)-2-oxo-3-{2,3′,6-trifluoro-4′-[5-(hydroxymethyl)-4,5-dihydroisoxazol-3-yl]biphenyl-4-yl}-1,3-oxazolidin-5-yl)methyl]acetamide (Compound No. 62),
N-[((5S)-3-{3-fluoro-4-[2-(1H-pyrrol-1-yl)pyrimidin-5-yl]phenyl}-2-oxo-1,3-oxazolidin-5-yl)methyl]acetamide (Compound No. 63),
N-({(5S)-3-[2,3′-difluoro-4′-(1H-1,2,4-triazol-1-yl)biphenyl-4-yl]-2-oxo-1,3-oxazolidin-5-yl}methyl)acetamide (Compound No. 64),
N-({(5S)-3-[3,5-difluoro-4-(6-{4-[(E)-(methoxyimino)methyl]-1H-imidazol-1-yl}pyridin-3-yl)phenyl]-2-oxo-[1,3-oxazolidin-5-yl}-methyl)acetamide (Compound No. 65),
N-[((5S)-3-{3,5-difluoro-4-[6-(4-formyl-1H-imidazol-1-yl)pyridin-3-yl]phenyl}-2-oxo-[3-oxazolidin-5-yl)methyl]acetamide (Compound No. 66),
N-[((5S)-3-{4-[6-(4-cyano-1H-imidazol-1-yl)pyridin-3-yl]-3,5-difluorophenyl}-2-oxo-1,3-oxazolidin-5-yl)methyl]acetamide (Compound No. 67),
methyl 1-[5-(4-{(5S)-5-[(acetylamino)methyl}-2-oxo-1,3-oxazolidin-3-yl)-2,6-difluorophenyl)pyridin-2-yl]-1H-imidazole-4-carboxylate (Compound No. 68),
N-({(5S)-3-[3,5-difluoro-4-(6-{4-[(E)-(hydroxyimino)methyl]-1H-imidazol-1-yl}pyridin-3-yl)phenyl]-2-oxo-1,3-oxazolidin-5-yl}methyl)acetamide (Compound No. 69),
N-({(5S)-3-[2,6-difluoro-4′-(1,3-oxazol-5-yl)biphenyl-4-yl]-2-oxo-1,3-oxazolidin-5-yl}methyl)acetamide (Compound No. 70),
N-({(5S)-3-[2,6-difluoro-4′-(5-methyl-1,3,4-oxadiazol-2-yl)biphenyl-4-yl]-2-oxo-1,3-oxazolidin-5-yl}methyl)acetamide (Compound No. 71),
N-({(5S)-3-[2,6-difluoro-4′-(2-methyl-2H-tetrazol-5-yl)biphenyl-4-yl]-2-oxo-1,3-oxazolidin-5-yl}methyl)acetamide (Compound No. 72)
N-({(5S)-3-[2,6-difluoro-4′-(1-methyl-1H-tetrazol-5-yl)biphenyl-4-yl]-2-oxo-1,3-oxazolidin-5-yl}methyl)acetamide (Compound No. 73)
N-({(5S)-2-oxo-3-[2,3′,6-trifluoro-4′-(1-methyl-1H-tetrazol-5-yl)biphenyl-4-yl]-1,3-oxazolidin-5-yl}methyl)acetamide (Compound No. 74),
N-({(5S)-3-[2,3′-difluoro-4′-(1-methyl-1H-tetrazol-5-yl)biphenyl-4-yl]-2-oxo-1,3-oxazolidin-5-yl}methyl)acetamide (Compound No. 75),
N-({(5S)-2-oxo-3-[2,3′,6-trifluoro-4′-(2-methyl-2H-tetrazol-5-yl)biphenyl-4-yl]-1,3-oxazolidin-5-yl}methyl)acetamide (Compound No. 76),
N-({(5S)-3-[2,3′-difluoro-4′-(5-methyl-1,3,4-oxadiazol-2-yl)biphenyl-4-yl]-2-oxo-1,3-oxazolidin-5-yl}methyl)acetamide (Compound No. 77),
N-({(5S)-2-oxo-3-[2,3′,6-trifluoro-4′-(5-methyl-1,3,4-oxadiazol-2-yl)biphenyl-4-yl]-1,3-oxazolidin-5-yl}methyl)acetamide (Compound No. 78),
N-[((5S)-3-{3,5-difluoro-4-[2-(1H-1,2,3-triazol-1-yl)pyrimidin-5-yl]phenyl}-2-oxo-1,3-oxazolidin-5-yl)methyl]acetamide (Compound No. 79),
N-{[(5S)-3-(3,5-difluoro-4-{6-[5-(4-fluorophenyl)-1H-tetrazol-1-yl]pyridin-3-yl}phenyl)-2-oxo-1,3-oxazolidin-5-yl]methyl}acetamide (Compound No. 80),
Hydrochloride salt of:
N-({(5S)-3-[4′-(1H-benzimidazol-2-yl)-2,3′-difluorobiphenyl-4-yl]-2-oxo-1,3-oxazolidin-5-yl}methyl)acetamide (Compound No. 81),
N-({(5S)-3-[2,3′-difluoro-4-(2-methyl-2H-tetrazol-5-yl)biphenyl-4-yl]-2-oxo-1,3-oxazolidin-5-yl}methyl)acetamide. (Compound No. 82),
N-[((5S)-3-{3,5-difluoro-4-[5-(2-methyl-2H-tetrazol-5-yl)-2-thienyl]phenyl}-2-oxo-1,3-oxazolidin-5-yl)methyl]acetamide (Compound No. 83),
N-{[(5S)-3-(3,5-difluoro-4-{6-[4-(hydroxymethyl)-1H-imidazol-1-yl]pyridin-3-yl}phenyl)-2-oxo-1,3-oxazolidin-5-yl)methyl}acetamide (Compound No. 84),
N-[((5S)-3-{3,5-difluoro-4-[2-(1H-pyrrol-1-yl)pyrimidin-5-yl]phenyl}-2-oxo-1,3-oxazolidin-5-yl)methyl]acetamide (Compound No. 85),
N-[((5S)-3-{3-fluoro-4-[2-(1H-pyrazol-1-yl)pyrimidin-5-yl]phenyl}-2-oxo-1,3-oxazolidin-5-yl)methyl]acetamide (Compound No. 86),
tert-butyl [((5R)-3-{3,5-difluoro-4-[6-(1H-1,2,4-triazol-1-yl)pyridin-3-yl]phenyl}-2-oxo-1,3-oxazolidin-5-yl)methyl]isoxazol-3-ylcarbamate (Compound No. 87),
tert-butyl [((5R)-3-{3-fluoro-4-[6-(1,3-oxazol-5-yl)pyridin-3-yl]phenyl}-2-oxo-1,3-oxazolidin-5-yl)methyl]isoxazol-3-ylcarbamate (Compound No. 88),
(5S)-3-{3,5-difluoro-4-[6-(2-methyl-2H-tetrazol-5-yl}pyridin-3-yl]phenyl)-5-[(isoxazol-3-ylamino)methyl]-1,3-oxazolidin-2-one (Compound No. 89),
(5S)-3-[2,3′-difluoro-4′-(1,3-oxazol-5-yl)biphenyl-4-yl]-5-[(isoxazol-3-ylamino)methyl]-1,3-oxazolidin-2-one (Compound No. 90),
(5S)-3-{3,5-difluoro-4-[6-(1H-imidazol-1-yl)pyridin-3-yl]phenyl}-5-[(isoxazol-3-ylamino)methyl]-1,3-oxazolidin-2-one (Compound No. 91),
(58)-5-[(isoxazol-3-ylamino)methyl]-3-[2,3′,6-trifluoro-4′-[4-phenyl-1H-imidazol-1-yl)biphenyl-4-yl]-1,3-oxazolidin-2-one (Compound No. 92),
(5S)-3-{3,5-difluoro-4-[6-(1-methyl-1H-tetrazol-5-yl)pyridin-3-yl]phenyl}-5-[(isoxazol-3-ylamino)methyl]-1,3-oxazolidin-2-one (Compound No. 93),
N-[((5S)-3-{3,5-difluoro-4-[6-(1H-1,2,4-triazol-1-yl)pyridin-3-yl]phenyl}-2-oxo-1,3-oxazolidin-5-yl)methyl]ethanethioamide (Compound No. 94),
N-({(5S)-3-[2,3′-difluoro-4′-(1,3-oxazol-5-yl)biphenyl-4-yl]-2-oxo-1,3-oxazolidin-5-yl}methyl)ethanethioamide (Compound No. 95),
(5S)-5-(aminomethyl)-3-{3,5-difluoro-4-[6-(1H-1,2,4-triazol-1-yl)pyridin-3-yl]phenyl}-1,3-oxazolidin-2-one (Compound No. 96),
methyl [((5S)-3-{3,5-difluoro-4-[6-(1H-1,2,4-thiazol-1-yl)pyridin-3-yl]phenyl}-2-oxo-1,3-oxazolidin-5-yl)methyl]carbamate (Compound No. 97),
methyl ({(5S)-3-[2,3′-difluoro-4-(1,3-oxazol-5-yl)biphenyl-4-3]-2-oxo-1,3-oxazolidin-5-yl}methyl)carbamate (Compound No. 98),
(5S)-3-{3,5-difluoro-4-[6-(1,3-oxazol-5-yl)pyridin-3-yl]phenyl}-5-(isothiocyanatomethyl)-1,3-oxazolidin-2-one (Compound No. 99),
(N-[((5S)-3-{3,5-difluoro-4-[6-(1H-1,2,4-triazol-1-yl)pyridin-3-yl]phenyl}-2-oxo-1,3-oxazolidin-5-yl)methyl]thiourea (Compound No. 100), or
N-[((5S)-3-{3,5-difluoro-4-[6-(1H-1,2,4-triazol-1-yl)pyridin-3-yl]phenyl}-2-oxo-1,3-oxazolidin-5-yl)methyl]-N′-methylthiourea (Compound No. 101).

9. A pharmaceutical composition comprising a pharmaceutically effective amount of compound of claim 1 or a pharmaceutically acceptable salt thereof and a pharmaceutical acceptable carrier.

10. A method of treating or preventing microbial infections comprising administering to a mammal in need thereof a compound of claim 1.

11. The method of claim 10, wherein the microbial infections are caused by gram-positive or gram-negative bacteria.

12. The method of claim 11, wherein the rain-positive bacteria are selected from staphylococcus spp., streptococcus spp., bacillus spp., corynebacterum spp., clostridia spp., peptostreptococus spp, listeria spp, or legionella spp.

13. A method of treating or preventing aerobic and anaerobic bacterial infections comprising administering to a mammal in need thereof a pharmaceutical composition of claim 9.

14. A process for preparing a compound of Formula X, 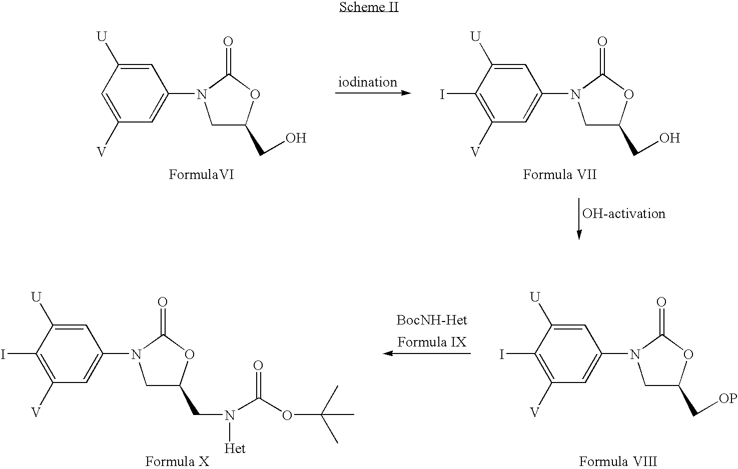
comprising the steps of:
a. reacting a compound of Formula VI with one or more iodinating agents to form a compound of Formula VII;
b. reacting the compound of Formula VII with one or more OH-protecting group reagents to form a compound of Formula VIII; and
c. reacting the compound of Formula VIII with a compound of Formula IX to form a compound of Formula X;
wherein Het is a heterocyclyl or heteroaryl,
P is a protecting group; and
U and V are independently selected from hydrogen (wherein both U and V cannot be H at the same time), lower (C₁₋₆) alkyl or halogen

15-17. (canceled)

18. A process for preparing compound of Formula XVIII, XIX or XIXa comprising the steps of: 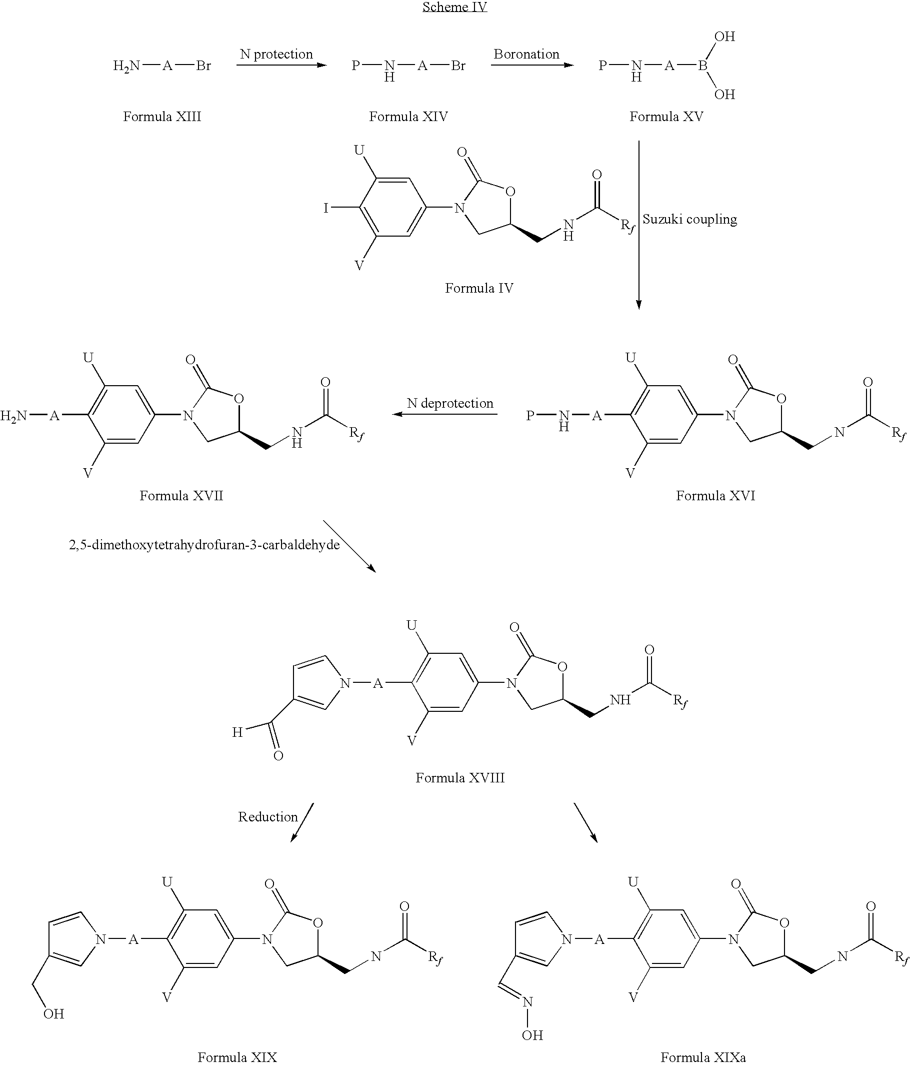
a. reacting a compound of Formula XIII with one or more protecting group reagents to a form compound of Formula XIV;
b. reacting the compound of Formula XIV with one or more boronating agents to form a compound of Formula XV;
c. reacting the compound of Formula XV with a compound of Formula IV to form a compound of Formula XVI;
d. reacting the compound of Formula XVI with one or more deprotecting agents to form a compound of Formula XVII;
e. reacting the compound of Formula XVII with 2,5-dimethoxytetrahydrofuran-3-carbaldehyde to form a compound of Formula XVIII;
f. optionally reducing the compound of Formula XVIII to form a compound of Formula XIX; and
g. optionally reacting a compound of Formula XVIII with hydroxylamine hydrochloride to form a compound of Formula XIXa, 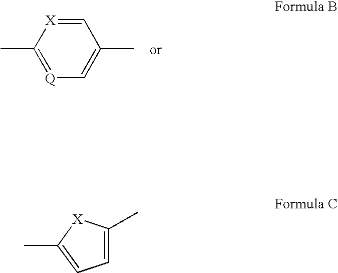
wherein A is
Q and X are independently selected from —O—, —C—F, —CH— or —S—;
U and V are independently selected from hydrogen (wherein both U and V cannot be H at the same time), lower (C₁₋₆) alkyl or halogen;
R_{f} is selected from hydrogen, alkyl, alkenyl, alkynyl, alkoxy, cycloalkyl, aryl, aralkyl, heteroaryl, heterocyclyl, heteroarylalkyl or heterocyclylalkyl; and
P is a protecting group.

19-28. (canceled)

29. A process for preparing a compound of Formula XXIV, XXV or XXVI, 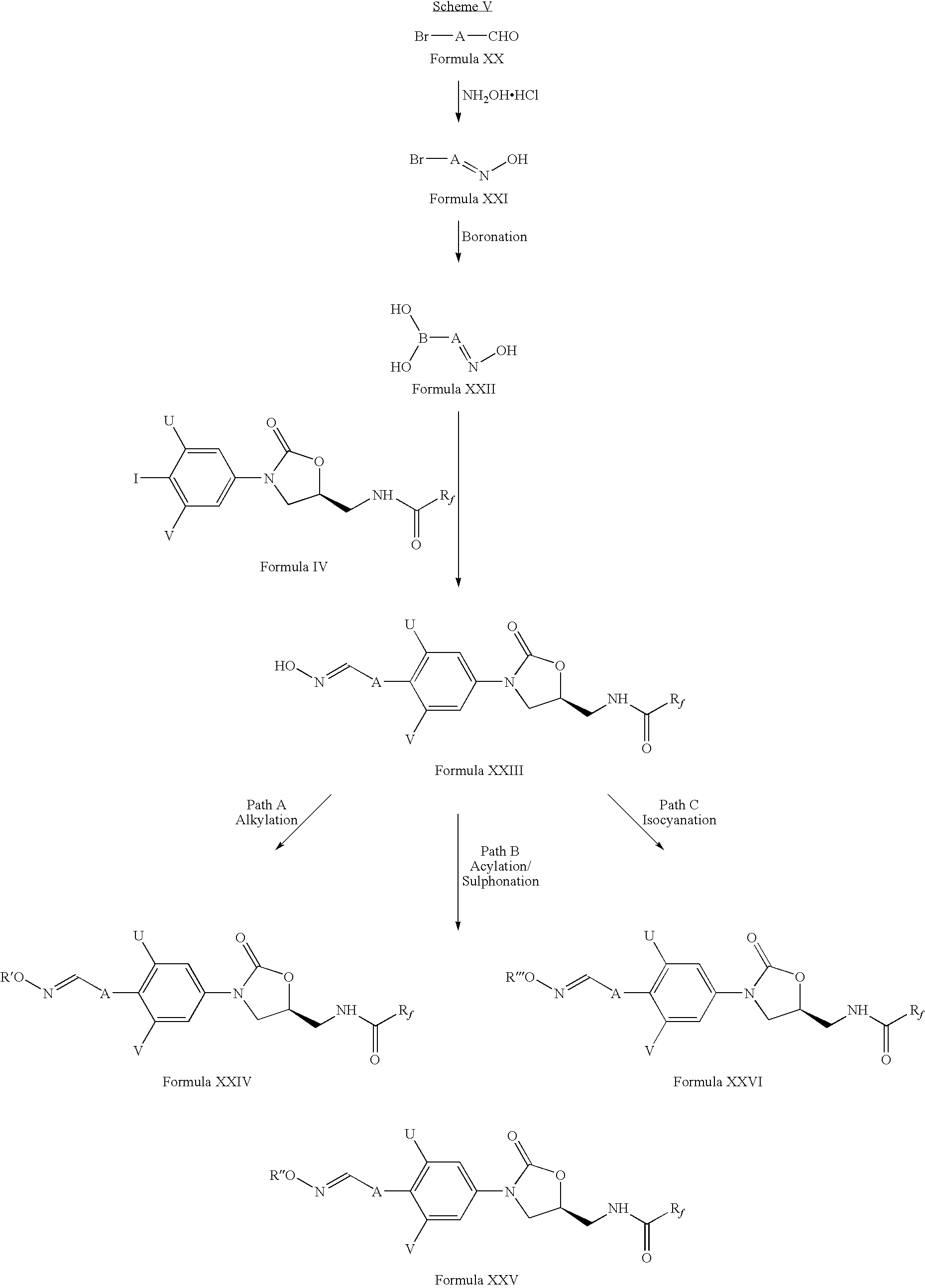
comprising the steps of:
a. reacting a compound of Formula XX with hydroxylamine hydrochloride to form a compound of Formula XXI;
b. reacting the compound of Formula XXI with one or more borating agents to form a compound of Formula XXII;
c. cross coupling the compound Formula XXII with a compound of Formula IV to form a compound of Formula XXIII; and
d. (i) reacting the compound of Formula XXIII with one or more alkylating agents to form a compound of Formula XXIV (path A);
(ii) reacting the compound of Formula XXIII with one or more acylating agents or one or more sulfonating agents to form a compound of Formula XXV (path B); or
(iii) reacting the compound of Formula XXIII with one or more isocyanating agents to form a compound of Formula XXVI, (Path C),
wherein A is 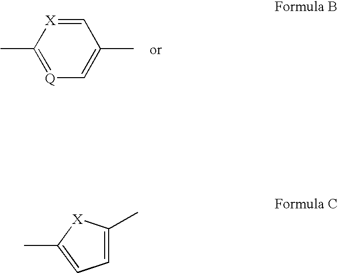
Q and X are independently selected from —N—, —O—, —C—F, —CH— or —S—;
U and V are independently selected from hydrogen (wherein both U and V cannot be H at the same time), lower (C₁₋₆) alkyl or halogen;
R_{f} is selected from hydrogen, alkyl, alkenyl, alkynyl, alkoxy, cycloalkyl, aryl, aralkyl, heteroaryl, heterocyclyl, heteroarylalkyl, or heterocyclylalkyl;
R′ is alkyl;
R″ is acyl or sulfonyl; and
R′″ is isocyanate.

30-40. (canceled)

41. A process for preparing a compound of Formula VII, 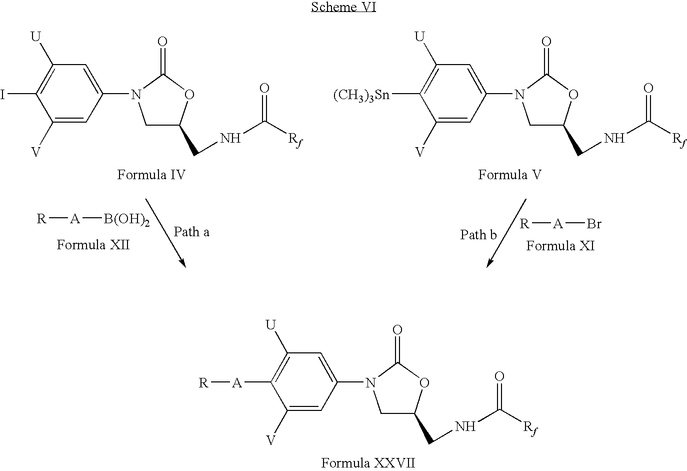
comprising reacting a compound of Formula IV with a compound of Formula XII to form a compound of Formula XXVII, (Path a); or
reacting a compound of Formula V with a compound of Formula XI to form a compound of Formula XXVII, (Path b),
wherein A is 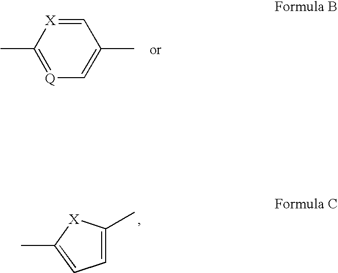
U and V are independently selected from hydrogen (wherein both U and V cannot be H at the same time), lower (C₁₋₆) alkyl or halogen;
R_{f} is selected from hydrogen, alkyl, alkenyl, alkynyl, alkoxy, cycloalkyl, aryl, aralkyl, heteroaryl, heterocyclyl, heteroalylalkyl or heterocyclylalkyl, and
R is CH═NOR_{f}, CH═NOC(═O)R_{f}, CH═NOSO₂R_{f}, CH═NOC(═O)R_{f}, CH═NOSO₂R_{f}, CH═NOC(═O)NHR_{f}, heterocyclyl or heteroaryl.

42-45. (canceled)

46. A process for preparing a compound of Formula XXVIII or Formula XXIX, 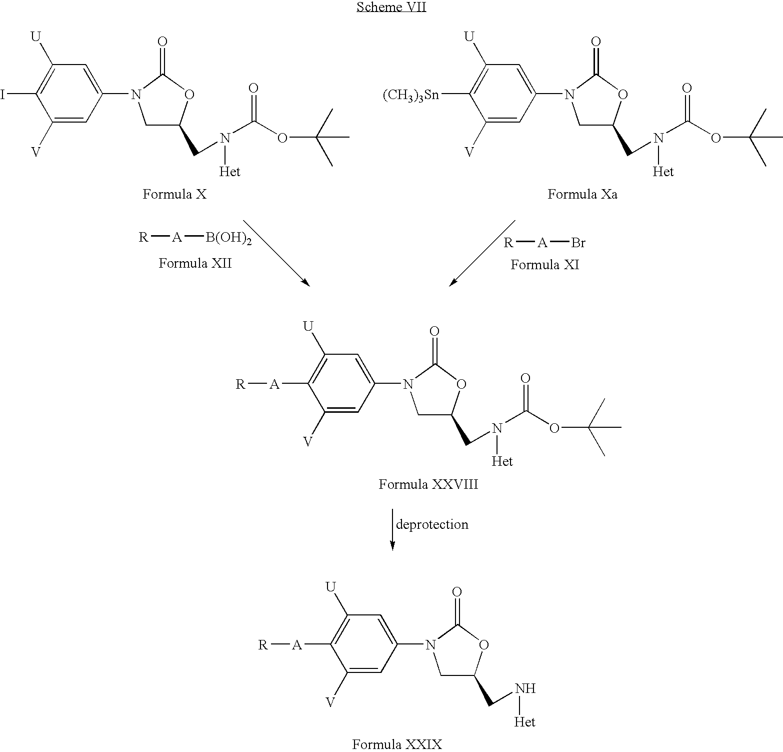
comprising the steps of:
a. reacting a compound of Formula X or Formula Xa with a compound of Formula XIII or Formula XI to form a compound of Formula XXVIII; and
b. optionally reacting the compound of XXVIII with a deprotecting agent to form a compound of Formula XXIX,
wherein A is 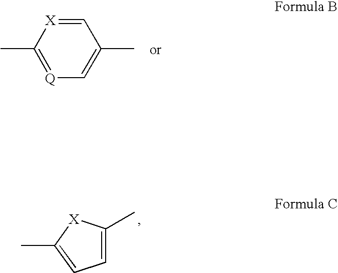
Q and X are independently selected from —N—, —C—F, —CH— or —S—,
U and V are independently selected from hydrogen (wherein both U and V cannot be H at the same time), lower (C₁₋₆) alkyl or halogen,
R is CH═NOR_{f}, CH═NOC(═O)R_{f}, CH═NOSO₂R_{f}, CH═NOC(═O)R_{f},
CH═NOSO₂R_{f}, CH═NOC(═O)NHR_{f}, heterocyclyl or heteroaryl;
R_{f} is selected from hydrogen, alkyl, alkenyl, alkynyl, alkoxy, cycloalkyl, aryl aralkyl, heteroaryl, heterocyclyl, heteroarylalkyl or heterocyclylalkyl, and
Het is heterocyclyl or heteroaryl.

47-49. (canceled)

50. A process for preparing compound of XXX, XXXI, XXXII, XXXIII, XXXIV or XXXV, 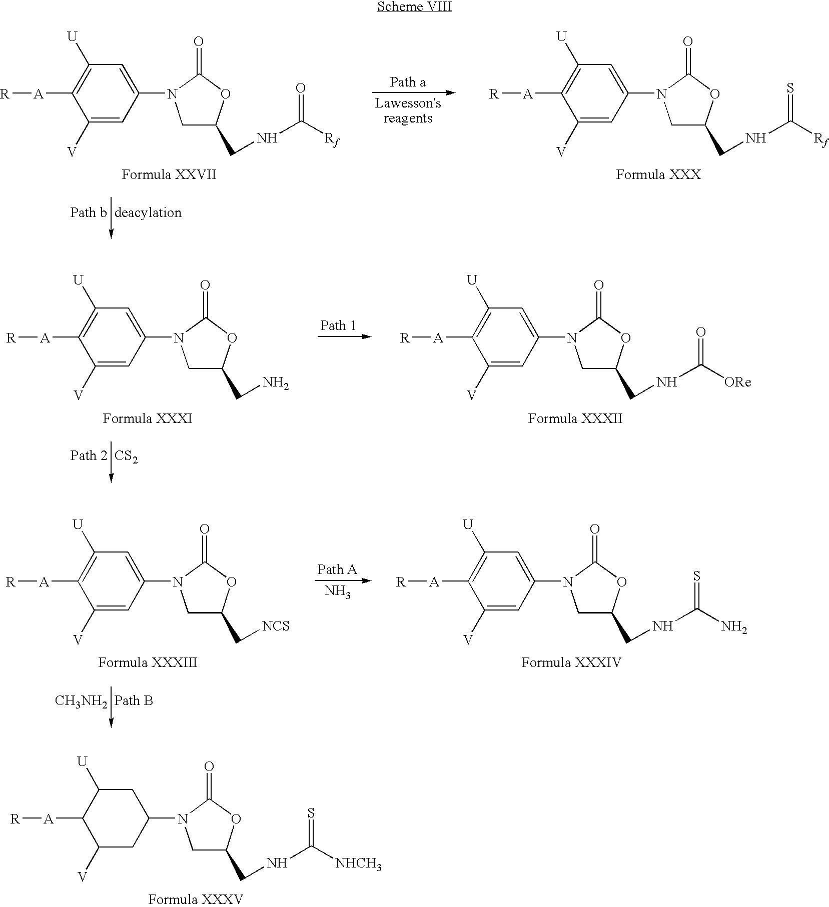
comprising the steps of:
a. reacting a compound of Formula XXVII with Lawesson's reagent to form a
b. deacylating the compound of Formula XXVII to form an amine of Formula XXXI, (path b);
i. optionally reacting the compound of Formula XXXI with alkylchloroformate to form compound of Formula XXXII (path 1); or
ii. optionally reacting the compound of Formula XXXI with carbon disulfite to form a compound of Formula XXXIII (path 2);
A. optionally reacting the compound of Formula XXXIII with methanolic ammonia to form the compound of Formula XXXIV (path A); or
B. optionally reacting the compound of Formula XXXIII with methylamine to yield the compound Formula XXXV (path B),
wherein A is 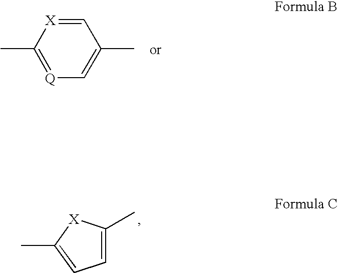
Q and X is independently selected from —N—, —O—, —C—F, —CH— or —S—,
U and V are independently selected from hydrogen (wherein both U and V cannot be H at the same time), lower (C₁₋₆) alkyl or halogen;
R_{f} is selected from hydrogen, alkyl, alkenyl, alkynyl, alkoxy, cycloalkyl, aryl, aralkyl, heteroaryl, heterocyclyl, heteroarylalkyl or heterocyclylalkyl,
R is CH═NOR_{f}, CH═NOC(═O)R_{f}, CH═NOSO₂R_{f}, CH═NOC(═O)R_{f},
CH═NOSO₂R_{f}, CH═NOC(═O)NHR_{f}, heterocyclyl or heteroaryl; and
Rₑ is an alkyl group.

51.-53. (canceled)
